SEQUENCE LISTING (159 sequences)

SEQ ID NO: 1 - DNA, 26174 bp, Homo sapiens
agcctcagccctccccccactttcctggctcccagccctgcctacctgaccctctcccttgctttgcgcccacttccctctctttctccccgacccttttgcccacccactctccctccttggctctgccctctagcccagaaggtctgaggcaatgggggcaagcttggagccgacagtgctgagcaggcaggagccaagagaggggaagcttgagcctcacgcagttaggggtgcgctggagagggtggggcccgactccgccacaccccaacggtccttccccctcctcaccactcccgcccccacccccaatggatctgggactgcccctttaagagtagtggcccctcctcccttcagaggaggacctattagagcctttgccccggcgtcggtgactcagtgttcgcgggagcgccgcacctacaccagccaacccagatcccgaggtccgacagcgcccggcccagatccccacgcctgccaggagcaagccgagagccagccggccggcgcactccgactccgagcagtctctgtccttcgacccgagccccgcgccctttccgggacccctgccccgcgggcagcgctgccaacctgccggccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcggtgagttcgcccaggtggctgcgtgcctggcggggagtggagagggcggcgggccggcgcccctggccggccgcaggaagggagtgagagggcctggaggccgataactttgccatagtctcctccctccccggaactgcccccagcgggtgactggcagtgtcaaggggaattgtcaagacaggacagagagggaagtggtggtctctgggagagggtcggggaggatataaggaatggtgggggtatcagggacaagttggggctggggccggcctgaattcggtcagattgggatttgccaactatttggagccggggggaggggcttgagcaaaacagaactagccctgccagctcgaagaactctgggcacccaggacacatcggagtggcagaaagggtcctgttagaactttgttagcgggcttggcactgtgctagctttgcccaagctggctctgaacacatgatgcccactaagacataactctcaagttggcatctgtccagcgtgttggagcgaggtcaggaaggcagggcaatcccccttttccctcccaagggcttggcggtggccccccctcagcatgaccttgtcctgggttctaagggttgggaagttctccctcactctgccactctgcgtgtctgggaccttccttgggctctgacaggcccaccaaaagagctccgggagatgagagatcggctcccccgcagctcccacagcccttggcctgcttggcccaggaatgcaagggagggagggaggcagagggcagaggctcccagctcaggaagttgtgttatgcccaggtctggccgcactcctcccttggccctctgcctagtgtcttcgagggttgggggcactgtccttccctccttggggtgagccactttcattttcccagcggggccaggcagtctttgctcgggcccatcctcttagctgctgacgttttgatctttgtcttattgaagtgctggaatacagtgacatttttgaaatccagccgttggaagattcaggccactcccactttacccacccctgccccaccctaccccaccctactcaactgcaccttcttcttttctaaaaaagcctttgggagcttggaagtataggccctctcttccagccccatcaaaatttgtttcccttcttcctgccttccctttctctatgcagacccaggccaagagcactaagggtgcttggagatccgtaaagggctgttggctttgacttcttctctctcttttatcatctactccaaacttctgctcttcctagaaccctttgctaggtgtggttttgttgcccaggctggagtgcaatggcacaatctcggctcactgcaacctccgcctcccaggttcaagtgattctcctgcctcagcctcccgaatagctgagattacaggcatgtgccaccatgccgggctaattttgtatttctagtagagatggggtttctccatgttcgtcaggctagtcttgaactcccaacctcaggtgatccacccgcctcagcctcccaaagtgctaggattacaggcatgagccaccacgctgggcccatcacccttctttctgaagagtcaatggaagttgtgtgtaggaagacaggcttaacggtttttttttgagacagggtcttactctgtcacccagactggagtgaagtggtgcgatcttggctcaccacaacctctgcctcccaggctcaaaagattctcctgcctcagcctcctgagtagctgggattataagtgtgtgccaccacacatggctatttttttttttttttttttttttaatttttagtagagatggggtttcaccatgttggctaggctggtctcaaactcctgacttcaaatgatccacctgcctcggcctcccaaagtgctgggattacaggtgtgagctaccatgcccggccatcaacctttattttgtttttttgagacggagtcttgctttgttgcccaggctggagtacagtagtgtgacctcaggtcactgcaacctctgcctcccaggttcaagccatgctcctgcctcagcctcccaagtagctgggactataggtgcctgccaccacgcccggctactttttatatttttagtagagacggggtttcaccatgttggccagggtgatctcgaactcctgacctcaagtgatctgcctgcctcagcctcccaaagtgttgggattagagacgggagccactgcgcctggcttctttttttcttgagatagggtttcactctgttacccaggctggagtgcagtggcaaggtcatggctcactgcagcctctacctctctggctcaagccatcctcccgcctcagcctcctgagtagctgggaccacaggcaggcaccaccacccacagctaatgtttttgtattattttgtagagatggggttttgccatgttgcccacagtcttgaactcctgggttcattctgctgaaagagaccacacctgtccttttctttatttttattatatttttcagagacagggccttgccctgttgctcaggctagagtgcaatggtacaatcataacttgctgcagcctggaactcctcctgggctcaagcgatcctaccgtctcaccttccggaatagctgagactaagggcaggcaccaccacgcttggctaatttttttttttttttttttttttttgctttttgtttgtaaagatggaaacttgctatgttgctcagctggttccgaagttttggcctcaagcaatcctcctgcctcggcctccggaagcactgggattacaggcataagccaccaggcctgacgccaggcctgtcttttttctactagtgatatgaacaatttagttagcaagacagataggaagcaaggaaggggagacccagagaattcgttgcattctaaactagtccactcatctaccaaagccctgtgaaggacatttttagcagttttagcagttttctggtcaaaactttgatcgagaaacagattgagtggattcgatattctcttgctcacccagccacgccagtttgtctcctctgcctcctagtgcagctgtccaggcctgggacaccaggcgggtatgtgcgcatgtggggcagggcggaggtggtgtgtgtacttgttatatttagccacctccctctgttctcccccactgatcctggctggaaaggctgggcttccggaaaagagaggtggatttgcacacctggatcccaagctgatagaaagtggggtgaagacaaaggggactcagactggggtgtctgtcctcttctatgcccacagtaggaggagccaggattggttactccctgctgggtctgctgtgctcagagtgaggtagagaagtgggtagagtaaagaatttgggagaggaaaaaaggcattttcccaacccctcccaccaaagcctagagagaaggtgttgtctggtttaatgtttaattagagctcagagttcagggccagatttggagttgggatggaaagttgtttttaagaccctgtagcaatttttgacccagcctgggtacctcaaccacactcaggagtttgggggaccttctgttgggctggattataggctccaagaagaaacccctttcgccaatactctctctctcttctttttttgagacagggtcttgttctgttgcccaggctggggtgcagtggcatgatcacagctcactgcaacgtcagcctcacaggctctggtgattctcccacctcagcctcctgagtagctgggattacaagtgtgtgccaccatgcccagctaattttttttttctttttttttttttgagacggagtcttgttctgttgccaggctggagtgcagtggtgcgatctcggctcattgcaacctccacctcccaggttcaagcgattctcctgcctcagcctcccgagtagctgggactacaggcacatgccatcacgcccagttaatttttgtatttttagtagagttggggtttcaccatgttggccaggatggtcttgatctcttgacctcgtgatccgtccaccttggcctcccaaagtgctgggattacaggtgtgagccaccgtacccggccactaatttttatatattttgtagagatggggtttcaccgtgttgcccaagctggtctcgaactcctaggctcaagtaatccacctgccttggccttggcctcccaaagtgctgggatgtataggcatgagctaccgcacctggtaccccctgccccttctctgtctctttctagtctgtagcccaagggatttggatacccaagtgcaggcagaatgggaaggttgtaagcaccagggaagcctgtctggagtccaggcttgcagctgggccccaccccaggcaaggcagctgggtggatgactcagatgctgcccccctccctcccaccctggtggctttacagaagacagcaggagacagggtggagacagcagttgtcttaaagggaggagtggtggtctgaatgtctacctcttctgcccccctccccattgcatcctggagtcccttgcctggctccttcctgagaccctctggtggtgtctggacacatagctctctctggacaggtaacatgcacaagtaattagaatccagagttgagttcagagttatggattgggctgcaggatagtgccagggtctgtgccttcccatgtgaaactgatggaggaaggctgagtcagaagtggggagatccgaggcccacaaagcagaagcgctacttccactccaaaaaggccctggtgcttgacaacttcctggattgcccactgttgcagccccagtgtggacaggcagggagatgcaggctccagttcatgtaggctctgatcaagacaagaacagcaaaggccacagaggcacagatgcttgtcccatgtcacacaataaaggggtcagcacttgatcacaggccttatgacttccagctgggtgtgctcttaccattaagcctcacttctctagcttgggggacaggttggagggaggatctagagggtgaggtaaggtgaagtcaggtagctgaggctcacttctgcagcctggaaactctgctctggggccagtgacaccttagtgctctatggccatacttcgtggctcatgcctgtaatcccagtgctttgggaggctaaggcaggaggatcacttgaggccaggagtttgagaccagtctgggcaacatagcaagacccccttctgtacaaaaaaattagccggtcaacacctgtagtccagctgcttgggaagctgaggcgggaggatcacctgaagccaggagtttgaggctattgtgagctatgactgcactactgcactctagcctgggagagagaaagaccctgtctctgaaaaagaaaaaaacaaaacaaaactctgctgtcctgcagggcctgttagcatatgatcgatagcctttgctccagcctatacctggacccaggacccctgccagcccctcaatcgtgagacggtcagagctctgggaggctggtgattcttgtcttgagactatcttgagacttgtcatgggaattgtccacccggattgaaaggaagctgtgccttttggcagacccattaggttaatggggttggagacctttgaggatgcatgggccctgggctttatctgagggtatctcctggtgttacctctccaaccctccaccaccaaatccattctttttttttttttttttttttttttgacagtctcgctccctggcccaggctggagtgcagtggcatgatcttggcttactgcaatctccacctcccaggctcaagtgatcctcccacctcagcctcccaagaagctgggactataggcacgtgccacatgctcggctaatttttctatttttagtagagaccaggtttcaccatgttactcaggctggtcttgaactctggggcttaagcagtccacccaccttgacctcccaaagtgctgagagccactgagcctagcccaaatccacgttctgattcaaagggaaagaagaagggtgcagctaaacctggggggtgagaagtacttaaaaagcccaagagaaacaaaagagagaataattcctcactaggaccccctattgccttcccactattggtgcccttgcttggcacttcccctggcctccaggagtctgagacttactcttccatggatgtgcccattgcccccacttccaggtccaccccccagtgattcggtagcttagtgtctgcgctgaagcccaggacagctggatggacaactggtagatcccttcacctaccaactgtgctttctgctcccctcccccttgcttccctcctccccagcccctcgccacccctagcagctgcagcagccaagaccaagtcttcagagacccagacacaagggcagggttcattccattctcacctccttggggtcccagtgtactgataggccgaactctaatattataggagatctctggaagattgcagggtctcttatccctcaataaggggcaaggcaagccgggcgcagtggctcacgcctgtaatcccagcactttgagaagccgaggggaacagatcacttcaggtcaggagttaagagaccagcctggccaacatggtgaaaccctgtctctactaaaaatacaaaaattaaccagaaatcgcttgaacccaggaggcagatgttgcagtgagccgagatcacgccactgcactccagccagggcgacagagcaagattccgtctcaaaaaaataatactaataataaataaataaataaggggcaaggtagtccaccaacaaaatgacaggcagtgtgatatagtggacaccctagccctcggtgcccttagttctgtgtgtggccctttcactaaattgctgtgtgaccttgagcaaatcgcctcccctttctggctttccttagctgtaaaagaaagggattggagcggaaagtctccagagaccttttaggttccaaagtagtacagtgacccacaaagtgagaaaacagtcttctaaaataccaagttattaatagtaaaatcaaatataaataatgtgaatatagttaatagctaatgttgttctcaatagaaatgtttcccacaagctgtggaattaaacatactaccacatttctctatttccccgtgaaagtttgttagaaatggttaaattgtgacattaccctcttggcaaatgttttgttttcattgctactaggaaagggcaactcgttttcgatgcctctcccttctggacggtggaaagggctgtgtcatagagtaggaacgggagatgcggcacaggaatggctcccattgacccgggttgggggctagggcgaaggcctaggagaggcagaactgttaccttagagctggccaggattagagaacagtgcctggaaccggggggaggggcacggtgaccttgggctgcccaccttctacccttccagcacccatactggctcccccaacctgcggctgggctgggaggaggtcttggcccctaccaatcccttaaggaaggggaaagagtttgggaaggggagtcctcccttcacccctgcctcccccaagttgtgagagaggaagccggaatcctgcctgctgaagccaggaataattctggctgagatcccaggcccggcaggggcgctgagtcatggtagagggcagagtggagagtggacaggagaccctaagcttgtccagtcagaaaagcagaggctgaggggtggccttttcttgagaactacattcaagttgcagcaagaaggacagtggtctgaatttgacggggacaaatggaagggagataggacacatgagttcctttaggtctggctcaggggagctagacttcatttcaaggggtctaggttctgggcagttgagaaggaggctatttggggtcaccaaggctcccctttcttcccaaagctctaacactgccaccttctgctggctaggagagagctgtgtcttctgaggctagagctggaatgcagtgagaccagactgcctaggtcctccctcacttcttctcctgaccttggggtgtggctcccactctctcccagtgtcctcagggttaataactatgtgccaccagatagagagttaaggggctgctgaattggcttcttgtgaagggaatcccctaaatgtccctcgttttggtcactggcctccctcccgcccccttcaggacattctactatcttcttaggccatccctccctcctccaggcactacttcttttgctctatccccaagccccacccctgcatttttgtgacaacaccggaatgatttctagagagagaggccaggaagaaggaaagtggcacttggcaggagaccttgcagggggcggctggtgaggaagccagccgcccattgtccaggaccccagtgccctggcctccggcctcaggcttctcctgcctctgtacaatgccacgttgatacgcccagcagctgtgactcaggcctggccccctgccaggcccagcacttctactggagttgcgtctgaacatgtcaacaggcttcctatccctctctcagcaccagttctccccacttcagcccctccctctgcctggaattaaaacctggctttgtcttagggaaggacagctgggagcctagtggctctggtaggggatctgagaggcctcagaccctaggcatatttggctgtttggcaggtgtcacgcccaagggaagcgtgtggaagcagagccatgcctgctgtgggtgcacatgcccgcgtgagggagtcggggtgtttcatcctggggcacctgtgggcttttgaggtgtatgatattcagaacttcacaggttggggtttggggaaggctcaaggggcttctaagtccctggaacagctgcccccctcagttcctctctctctctctctttttttttgagatggagtctcgctctgttgcccaggctagaatgcagtggcgcgatcttggctcactgcaaactccgcctcctgggttcaagtgattctcctgcctcagcctcccaagtagctgggactataggtgcccgccaccatgcctggctaatttttgtatttttagtagaaatggggtttcaccatgctggccaggatggtctcaaactcctgacctcgtgatccacccaccttggcctcccaaagtgctgggattacaggcgtgagccactgcgcccagcctcagttcctctctttaaggtctcctttccagagagggatagcacctcaaatgccagggaggggaattctccacatcctgcccttacccgagttgtggcagacccacagactagccaagaaaccaagcagtggttactttgccgggttgggggggaggtaggggctatcaaacctcatgattggccgcacacaaaggtgtgagtatgtgtatatttgagggtgggtgggagtggcactttcactaggcctccgtatcactctctgactggggtatctcccagcaagcgagacagaggcagacacgcttcccagactgtcttactgggtctctctgtgttattctctgcagtgtctgtgtgtatcgtgccattttctatgttttgcaccaatctgctgtgagtgtcctcaggtgacctgggggcaggtttttagtgcctgagcctacccgtctccaggctttagtttccccctgtaaaagtataggagttggttcaagagaaggttcctctagaagccttgagcctgtgaaccgtctagtctccgggtatttgtgggacacacagaaaaagccccacgacccaacaggtagaacactggctgaaatcagcagggcagagctgagacaggctcaagtaggctgaggggtagggaggttttgggtgaatgggagggagggacagagagaaggaggatatattgcagtaggaggagttgctggaacaaaaggaggggtggtaggagtggcttggggtggcagcagaagacgccctgtcacatggcgggaagtcagcctgggcagaggtctaggtgtccaggaggggctgggtgtggtggctcacgcctgtaatcccaggactttgggaggctgatgcaggaggatcacgtgaggtcaggagttcaagaccagcctggccaacatggcgaaaccctatctctactaaaaatgccaaaaattagctgggtgtggtggcaggcgcctgtaatcccagctactctggaggctgaggcacaagaattgcttgaacctgggaggtggaggttgcagggagccgagatcgcgccactctactctagcctgggcaacacagtgagactctgtctcaaaaataataataataggggctgggcgcggtggctcatgactgtaatcccagcattttgggaggtggaggcgggtggatcacctgaggtcaggagtccgagaccagcctggccaacatggcaaaactccgtctctactaaaaatagaaaaattagctaggcatggtggtgcaggcctgtaatccagctactcgggaggctgagaagcaggagaatcacttgaacctgggaggtgaaggttgcagtgagatcacctgggcgacagaatgagactccacctcaaaataataataatagtaataataataaatgaaaaattttaaaattaaacaattaaaaattttaaattaaaattaaacaaattagatgcccaggaggatacaggagagcatttgccaccaggcggactccctgtacccacccggccacagggggcgatgttcctgggagacaggaaatgcccaggggctgggagaccctctgctcttctgctcccttcctgtgtgctgcctggcaatggggaactctgagggctggtgagcagggctgctgaggagtgggtctaaggagtccctgcagggctgggccagctcctccacctcccctttgtcttcccctcccacttgttatttttagctacagtgtctgtccctcttgcttctcccccagattgggagaggaaacggaggcctctccctccgggcctagcctgttgcccccagcaaccgggcccaaacaggcctgtggccggccctggcttccatatctggcatcagagttgggctgagcagggtgactcagagggtgggtcagcgcctggcccggtgcccacctagcccctttgctgtgctggtgcctttcttccccaaacagccccaagggcccgggcctgctgcagctggggagccggacttccttgtcccaccaggcacagctcttcagacccctgccttgggtcacatttgcaagtgccaactctcatttctaccttattcttttcctctctgttcccctccccaccccctctcttccctctttctgagatcagatttgccagtgatgggaagagttagaaacaggatgcccagcccttctcgcctcaagaggccactgggatgcagccactcctgtgcttggggaacctggaggatgcaagggaaaggactggcactctgctggcacagcacccggcctggggcaggacacgggcgaagccagggtctcccctgtgagcactagaggatttcccgacccctgcccgggtattgtgtgcctgagcatgagtcacctgaggggcccaggttcccacccttcccagctcctctggcctgccccaccctgtcctccctgccaacccagcacggggacggcactcagcgtgtgctcagctttcctgatgccaacccccagtggagtgggctgcaccaccaccctgggaccgaatgcctggctagggtctactttggtccctgctaggtctgaggacccctcctaggaaggaaatggcacttgggggcgggggcagggaggagggaggagagacactgggctctactgtacccctagtcatctcttggggtgtgcgtgtggctccctggccacagagctcccaaggtctgagtcatgagcccatgggtgatagtggcttcttccccgcagatgggagctccccgtgcctaagaaaaccacaaaggttcttcctcacttccctctctgctcgtggtttttctcatctgcagggtgtgtcttagtcctttaatctcctctctttgcagtgctagtcaaaacctccaccagggaaagacaaataacccccttactgtttttttttttttttttttttttttttgagatggagtctcgctctgtcacccatgctgtagtgcagtggcacaatctcggctcactgcaacctccgcctcccaagttcaagtgatcctcctacctcagcctcctcagtagctgggactacaggtgcacaccaccgtacccagctaaattttttttttttttttttgagatagagtctcactctgtcacccaggctggagtacagtggtacaatctcaactcactacaatctccgcctcccaggctcaagcaattctcgtgtctcagcctcccaagttgctgggactatggacgtgcaccaccttgcccgactaatttttgtatttttgatagagtcagagtttcaccatgttggcaggctggtctcgaactcctggcctcaagtgatccacctgccttggcctcccaaagtgctgggattacaggtgtgagccaccacactcagccagcccccttactttccttggagaccatatactgtggcttgtgccaaagtggtacagcatggatttccagctcccctatctacttgctgcgggaccctagatatagctttctgtgcctatttcctcaattgcataggaatagcacctatcgcatagggtagctgtgaagatgacgtgagttaacataatatttagagcagtgcttggtacctaataagctctatataagtgtttgctattatattattattatcactgccaccaccgcttttgcaagcagcagaaggtgaagaggttagactgaagaaaaaacttctgtgctcatcagcccataagctcgcagagcacagggatcatgcatctatgttttcctcagtcagtgtctgccaggcactggcaaggaaaggctgttaccagggggaactccaggaattcctcctggcacctaaggaggctggggagacaggactagggaaaaggtgcccttgagacaccttctgaaatcatcccattgccttccagcttctttcagctcaggctggctggtcagggaaacgctttgtgccatagtgtctgccctcttcctcctcctggcttctccattctctctggaacttgtggcttaggaaagcagtgaggtggaggaggaggaaccctagatcagcagctagaattgactggaatgctgctgctggctttcggtaattgacactgggccattcaccttcctcctttgcacctcagtttcctcatctataaaagggagagggttgagctgaatcaactctaagctccttctagttctctaaattctgagagcctcctagtacagccagcagcagccattagccttcagggtagagaggcctcttctgggaagccccagccagcctgggggtcagcccaaggagctcggaatctaagttgccccagttgcttcactttaccagcggtttttcttcattttccctcctccccctgcagctgcttcagcttcggaaaagttctgaagtcatggaaagttggggctgtgctcccagccaggggctaggccggatggcagccaaaacctgagctgggttttgactttatttttagcttttctgactgagacagaggagggaatacattctccggttctggaaggggctcttttttgcaggagacagacacttacattaaacaacttgttctgaggtgtggccagaggcctggactgagcaagtgtgcaggctgggggagcttcctctggcttctcatgtccttcccctgcccctctgagtgtcactctatcctcctccctgcctggtggggggaggtgggggtgactccttttttggactctcctaagcagaacactgcctgggtctcgtcctccagagcttctgcaaatctagccttccctatccctcttcacagtgaattgctgggcctcttggagtttaggacttttgtggtagaagaaaaatgttggcagggctgcttttctcctttccaggatagatttttccttctgcccacgcttggttttccttttttccatctgctgtggtgggctcatgcttaagcactgatgagttacagatggcagctggaaccaggtcctctggatctttccctccgctccctgggtctgctgctttctctcaccctatatttgtgaagcaattgtaacatctagaaagttcttgggttctctggaggtttttaagaaaataggacctttctatttctccagtccactagcaaaaataatcaggggcccagaaaaggtgagggaggtggcagaggcagcgctgttcgactggttatagctaaagctttacccactttgaggagcagggaggcttaaagctggggcccagatggacctggaggcctgggatccacatctggaaccagatgctgaggctatggtagatgggtagggctcagccttctcccagggcacggatgaggcaggagggagggaggcagggacccctctgttcagtgcagatcagggcacccagactgggtcctgagaaaggaaagggtcaatattgtgcctggtcatccttgtctgaggtccctctgagctctaaccagactttccttccccacagtcccacatgtgtaaaagggactaggagaggtgaccagtacctttggggctcagatcgagaagtgctagggacatgtgggccatgagcttagttgtcaggctcctcagagggagggaagcttggccaaagggaagtgagtagagtccagggagaaggctaagtaaggccctgtgtgggaaggggcaggagacaaaggtacccctgtctctttgggaaagaatgggaggagagagagggaaaagcattcatatcacggggtagagctctgcccttggccccaggcacgttcctgagccctgagtcatgggaagggtggagaagcaggaagggggttttcaaggaccttggggaggtgggagcccagccccagaggcaagcagatgcaaaccaacctaatgcaaggatgccctctcctggtaattgcaggcatagcagcgccagcccccatggctgacctcctgggagcctggcactgtctaggcacacagactccttctcttaaatctactctcccctctcttctttagcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaaggtgaggccaccctgcagggcccacccatggccccacctaacacatgtacactcactcttctacctaggccctcccccatgtggtgcctggtctgacctgtcacctgatttcagagccattcacctgtcctagagtcattttacccactgaggtcacatcttatcctaatttggctgccaatgggatctaccacagtgaatttaaaataatccaggaggccgggcatggtggttcacgcctgtaatcccagcactttaggaggccgaggtgggccgatcacgaggtcaggagatcgagatcatcctgactaacatggtgaaaccccgtctctactaaaaatacaaaaaattagcctggcatggtggcgggcgcctgtagtcccaactactcgggaggctgaggcaggagaatggcgtgagcctgcgaggcagagcttgcagtgagctgagatcatgccactgcactccagcctgggcaacagagtgagactccgtctcaaaaaaataataataataataataaaaataatccaggccatgtgtggtggctcatgcctgtaatcccagcattttgggaggccaaggaggcaggattgcttgagtccaggagtttgagaccagcctgggcaacacagaccccatctctagaaaataaaaatttaaagaaattagctgggcatggtggtgtgcacctatagtcccagctacttgggaggctgaggcaggaggatggcttgaacctgagaggtcgaggatacagtgagctgtgattgcaccactgcacttcagcctgggtgacagagggaaaccctgtctctacataaataaatacataaaataaaataatccacaagccatttctacttaactttgcaatgaactgtacctgaccctagatccctcccagtttggccctccggtatacaagggcctcctataggcccttgtgatttctctggggaaaaggaggactggagttgatcatttattgaggccatcagaagcggatggctaattacatatgggacatgtgttaataatgctttgtgtatatagagtggcctttactttcaaaacactcttctccaatttatcatgttaaaagctaggaattgggctgggtgcagtggctcacgcctataatcccagcactttgggaggccaaggcgggtggatcatttgaggtcaggagtttgagaccagtctgaccaacatggttaaactccgtctctactaaaaatacaaaattagccaggcgtggtggcacacacctgtagtcccaacaactacttgtgaggctgaggcaggaaaatcatttgaacccaggatcagaggttgtggtgaactgagattgcaccattgcactccagcctgggcaacaagagcaaaactctatctcaaaaaaaataaaaaatagccaggcacggtggctcatgcctgtaatcctagcactttgggaggcagaggtgggcagatcacctgaggttaggagttcgagactagcctggccaacatggtgaaaccccatctctactacaaatacaaaaattagctaggcatggtggcagccacctgtaatcccagctacttgggaggctgaggcaggagaatcgcttgaacccgggaggtggaggttgcagtgagccaagatcgggtcacagcactccagcctaggcaacagagcgagactccatctcaaaaaaacataaataaataaaaataaaaataaataataaataaaagctaagaatcaaagaagcagtttattcctaatttcacagtctcatctgttcatagtggggccaggattagagtcagtggccaagcttccatcctgggttctttcccttcccaggccctaccatcatagtataccagggaaagacctggagaagccagcaggttgaccaccgaaccaaggctgggccaccttcctcctgggtctggtctccagcctcccagttgtacccttcccccagcccttcctggatgcactgatcagcctgtgcttccttgccctgtttttctttataaatagagccatgttctcctctctctctctctcttttttttttttttttttttgagatggagtcttactctgtcacccaggctggagtgcaatggcacgatctcagctcactgcaacctctgtctcccaggttcaagcaattctcctgcctcagcctcccgagtagctgggattacaggtgcccaccaccatgcccagctactttttggatttttagtagagacagggtttcaccatgttggtcaggctggtcttgaactcctgaccttaggtgttctgcccgcctcagcctcccaaagtgctgggattacaggcgtgagccaccacgcctggcaagacgtgttctctctatgttgttgaggctggtcttgaactcctggctgcaagagatcttcctgcctcagcctcccaatgtgctgggattataggcatgagccaccacacttagcccagcctgtgctttcttaaatgaaaatctaagcatacggctgggtgtggtggctcacgcctgtaatcccagcattttgggaggccaaggtgggcagatcacgaggtcaggagatcgagactatcctggccaacatggtgaaaccctgtctctactaaagatacaaaaattagctgggtgtggtggcccatgtctgtagtcccagctactcgggagactgaggcaggagaatggcatgaacctgggaggcagagcttgcagtgagctgagatcgcgccactgctctccagcctaggtgacagagcgagactccatctcaaaaaaaaaataaaaataaaaaaaagaaaatctaagcgtggtgctcccctgctcaaacatcctcaggttcttttcatggcagataagggcatctcttcatgagccagcccctgcctactgacccagccacctctcccatcccttcccaccccgtacttcaggcttcagcagtactgatctttccaaagaccccagaacacacatgccttcatacctctgtgcctgtacatgcttgtttctgcccttgaaatcatgacagtagctctctgtaggccccgctagcctgtcccttgggtcttagcctcttggaggccttcccagagccccccaaaagtaccccaggcatactttggttccttctctcatgtcccctcagtactttgcacatacctcctttatagcagttgctatgttgtgccagagaagggagtcctgtggctggggggcatatatcttttctttttgagacagagtctagctgtgtcacccaggctggagtgcagtagtgcgatctcggctcactgcaacctccacctcctggattcaagcgattcttgtgcctcagcctcctgagtagctgggactacaggcgtgtgccaccatcatgcctggctacttttttgtattagatatatattttctctcttagcacagtacctaccaagagtgagtgagtagatgtcctgacccctgcaggcatccaaggccctccttccctggacctgtttccacatgtgtgaaggggtgcacaggcagcagcccacctctcagcttccttccagttcttgtgttctgtgaccccttttcctcatctctgcctgcttcctcacagcttgaggcagccctaggtgaggccaagaagcaacttcaggatgagatgctgcggcgggtggatgctgagaacaggctgcagaccatgaaggaggaactggacttccagaagaacatctacagtgaggtggggactgtgctttgcaagccagagggctggggctgggtgatgacagacttgggctgggctaggggggaccagctgtgtgcagagctcgccttcctgagtcccttgccctagtggacagggagttgggggtggccagcactcagctcccaggttaaagtggggctggtagtggctcatggagtagggctgggcagggagccccgcccctgggtcttggcctcccaggaactaattctgattttggtttctgtgtccttcctccaacccttccaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaaggtgcttgctctcgattggttccctcactgcctctgcccttggcagccctacccttacccacgctgggctatgccttctggggatcaggcagatggtggcagggagctcagggtggcccaggacctggggctgtagcagtgatgcccaactcaggcctgtgcctccacccctcccagtcaccacagtcctaaccctttgtcctcccctccagctggacaatgccaggcagtctgctgagaggaacagcaacctggtgggggctgcccacgaggagctgcagcagtcgcgcatccgcatcgacagcctctctgcccagctcagccagctccagaagcaggtgataccccacctcacccctctctccaggggcctagagtctgggccggatgcaggctggaagcccagggttgggggtgggggtgggggtgggaggttcctgaggaggagagggatgaaaagtgtccccacaaccacagagaagggtcgcaggatgtggagtcagatggcctgtgtgctgtttctgtacactcttacctcaccttcacttctcagggctttggttttcccattcgaaaatggaggctgttcttaatctccctaactcagagttgccacaggactctgcaatgtgaggtgttaaaagcatcagtatttttctagttggctgtgctatttgtgacaggagaaaaagtctagcctcagaacgagaggtttcagttagacaaggggaaggacttcccagttgccagccaagactatgtttagagcttgtgatgttcagagctggctctgatgagggctctggggaagctctgattgcagatcctggagagagtagccaggtgtctcctacaccgacccacgtccctccttccccatacttagggcccttgggagctcaccaaaccctcccaccccccttcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggtgggctggggagacgtcggggaggtgctggcagtgtcctctggccggcaactggccttgactagacccccacttggtctccctctccccaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggtaggctcctgctcagggtctaaggggatacagctgcatcagggagagagtggcaagacagaaggatggcatgtggagagaggaacatccttgccctcagagggtggaccagggtgagcctgtatatctcctccacactctggttccaggcctggctcctggactctttggctgtgagaccttgagcaggttatttaacctctcagagcatcagtttcctcatctgtaaaatggggatgaatactgatccctaagtctttgagttgtcaggaagatgaaagataaggtatccgtgtgcctggtgctgcgtatgtgtccacagatcatggctattatccccgggggaagggcagtgacaggggtgtgtgtagatggaaggagaggcctcaattgcaggcaggcagagggctgggcctttgagcaagatacacccaagagcctgggtgagcctccccgaccttcctcttccctatcttcccggcaggaccagtccatgggcaattggcagatcaagcgccagaatggagatgatcccttgctgacttaccggttcccaccaaagttcaccctgaaggctgggcaggtggtgacggtgagtggcagggcgcttgggactctggggaggccttgggtggcgatgggagcgctggggtaagtgtccttttctcctctccagatctgggctgcaggagctggggccacccacagcccccctaccgacctggtgtggaaggcacagaacacctggggctgcgggaacagcctgcgtacggctctcatcaactccactggggaagtaagtaggcctgggcctggctgcttgctggacgaggctccccctgatggccaacatcggagccagctgcccccaacccaagtttgccaattcagggcccctttctagagctctctgttgcaggctccagacttctccacccagtaggcaaaccaaaagatgcttcctcaacagcacaaggggtggaagttagacagtgaggattgttaaaggcagagccatactcctacccggagagcttgacagtgtccctctggggtggaaatgagttccttagctccatcaccacagaggacagagtaagcagcaggccggacaaagggcaggccacaagaaaagttgcaggtggtcactggggtagacatgctgtacaacccttccctggccctgacccttggacctggttccatgtccccaccaggaagtggccatgcgcaagctggtgcgctcagtgactgtggttgaggacgacgaggatgaggatggagatgacctgctccatcaccaccacgtgagtggtagccgccgctgaggccgagcctgcactggggccacccagccaggcctgggggcagcctctccccagcctccccgtgccaaaaatcttttcattaaagaatgttttggaactttactcgctggcctggcctttcttctctctcctccctataccttgaacagggaacccaggtgtctgggtgccctactctggtaaggaagggagtgggaactttctgatgccatggaatattcctgtgggagcagtggacaagggtctggatttgtcttctgggaaagggaggggaggacagacgtggggcatgcccgccctgcctctctcccccattcttgttgcatgcatatcctctcatttccctcatttttcctgcaagaatgttctctctcattcctgaccgcccctccactccaattaatagtgcatgcctgctgccctacaagcttgctcccgttctctcttcttttcctcttaagctcagagtagctagaacagagtcagagtcactgctctggttctctgtccccaagtcttcctgagccttctccccttttatgtcttccctctcctcctccgggcccctagcctcccaaacccccattgcccgctggctccttgggcacagaaccacaccttcctgcctggcggctgggagcctgcaggagcctggagcctggttgggcctgagtggtcagtcccagactcgccgtcccgcctgagccttgtctcccttcccagggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccaggtgagttgtctctgctttgtctccaaatcctgcaggcgggtccctggtcatcgaggggtaggacgaggtggccttgcaggggggagagcctgccttctcttccgcagcccgggggagtgggagcctcctccccacagcctgagtcctagacagcccacctctgcatcctgcccctcttgtctgagccccagactggagggcaggggcagggctggagtgtgagggatgggggagatgctacctcccttctaggggccaggggagggagggtctgggtccaggccctgctgctcacacctctctcctctgttttctctcttagagcccccagaactgcagcatcatgtaatctgggacctgccaggcaggggtgggggtggaggcttcctgcgtcctcctcacctcatgcccaccccctgccctgcacgtcatgggagggggcttgaagccaaagaaaaataaccctttggtttttttcttctgtatttttttttctaagagaagttattttctacagtggttttatactgaaggaaaaacacaagcaaaaaaaaaaaaaagcatctatctcatctatctcaatcctaatttctcctcccttccttttccctgcttccaggaaactccacatctgccttaaaaccaaagagggcttcctctagaagccaagggaaaggggtgcttttatagaggctagcttctgcttttctgccctggctgctgcccccaccccggggaccctgtgacatggtgcctgagaggcaggcatagaggcttctccgccagcctcctctggacggcaggctcactgccaggccagcctccgagagggagagagagagagagaggacagcttgagccgggcccctgggcttggcctgctgtgattccactacacctggctgaggttcctctgcctgccccgcccccagtccccacccctgcccccagccccggggtgagtccattctcccaggtaccagctgcgcttgcttttctgtattttatttagacaagagatgggaatgaggtgggaggtggaagaagggagaagaaaggtgagtttgagctgccttccctagctttagaccctgggtgggctctgtgcagtcactggaggttgaagccaagtggggtgctgggaggagggagagggaggtcactggaaaggggagagcctgctggcacccaccgtggaggaggaaggcaagagggggtggaggggtgtggcagtggttttggcaaacgctaaagagcccttgcctccccatttcccatctgcaccccttctctcctccccaaatcaatacactagttgtttctacccctggctgctgtggtgtctttgttggtggacgtcgctgtgtgtactgaggtgcagactcgtgggcatgcgcgcgcgtacacacacacacacacacacacacacacacacacacacacacacagcgccgcacggtcactgcatcctcctgctcattgctgcccagccctgccctgctccaggggaaacaattagagatcagagcactttgggtgcacatctgggcactcggtggtggctgagggggaaggctttgatataaccatcaccacccactgtacggctctctcccaacggaagtccttcagagaagcagcacctgaaggagggcatttgctaactaacttccctcgtccaccagcacttactgagtgctttctctgtgctgggccctgagcg

SEQ ID NO: 2 - DNA, 3181 bp, Homo sapiens
actcagtgttcgcgggagcgccgcacctacaccagccaacccagatcccgaggtccgacagcgcccggcccagatccccacgcctgccaggagcaagccgagagccagccggccggcgcactccgactccgagcagtctctgtccttcgacccgagccccgcgccctttccgggacccctgccccgcgggcagcgctgccaacctgccggccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggcggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgtaatctgggacctgccaggcaggggtgggggtggaggcttcctgcgtcctcctcacctcatgcccaccccctgccctgcacgtcatgggagggggcttgaagccaaagaaaaataaccctttggtttttttcttctgtatttttttttctaagagaagttattttctacagtggttttatactgaaggaaaaacacaagcaaaaaaaaaaaaaagcatctatctcatctatctcaatcctaatttctcctcccttccttttccctgcttccaggaaactccacatctgccttaaaaccaaagagggcttcctctagaagccaagggaaaggggtgcttttatagaggctagcttctgcttttctgccctggctgctgcccccaccccggggaccctgtgacatggtgcctgagaggcaggcatagaggcttctccgccagcctcctctggacggcaggctcactgccaggccagcctccgagagggagagagagagagagaggacagcttgagccgggcccctgggcttggcctgctgtgattccactacacctggctgaggttcctctgcctgccccgcccccagtccccacccctgcccccagccccggggtgagtccattctcccaggtaccagctgcgcttgcttttctgtattttatttagacaagagatgggaatgaggtgggaggtggaagaagggagaagaaaggtgagtttgagctgccttccctagctttagaccctgggtgggctctgtgcagtcactggaggttgaagccaagtggggtgctgggaggagggagagggaggtcactggaaaggggagagcctgctggcacccaccgtggaggaggaaggcaagagggggtggaggggtgtggcagtggttttggcaaacgctaaagagcccttgcctccccatttcccatctgcaccccttctctcctccccaaatcaatacactagttgtttct

SEQ ID NO: 3 - DNA, 3089 bp, Homo sapiens
aggaggacctattagagcctttgccccggcgtcggtgactcagtgttcgcgggagcgccgcacctacaccagccaacccagatcccgaggtccgacagcgcccggcccagatccccacgcctgccaggagcaagccgagagccagccggccggcgcactccgactccgagcagtctctgtccttcgacccgagccccgcgccctttccgggacccctgccccgcgggcagcgctgccaacctgccggccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccagagcccccagaactgcagcatcatgtaatctgggacctgccaggcaggggtgggggtggaggcttcctgcgtcctcctcacctcatgcccaccccctgccctgcacgtcatgggagggggcttgaagccaaagaaaaataaccctttggtttttttcttctgtatttttttttctaagagaagttattttctacagtggttttatactgaaggaaaaacacaagcaaaaaaaaaaaaaagcatctatctcatctatctcaatcctaatttctcctcccttccttttccctgcttccaggaaactccacatctgccttaaaaccaaagagggcttcctctagaagccaagggaaaggggtgcttttatagaggctagcttctgcttttctgccctggctgctgcccccaccccggggaccctgtgacatggtgcctgagaggcaggcatagaggcttctccgccagcctcctctggacggcaggctcactgccaggccagcctccgagagggagagagagagagagaggacagcttgagccgggcccctgggcttggcctgctgtgattccactacacctggctgaggttcctctgcctgccccgcccccagtccccacccctgcccccagccccggggtgagtccattctcccaggtaccagctgcgcttgcttttctgtattttatttagacaagagatgggaatgaggtgggaggtggaagaagggagaagaaaggtgagtttgagctgccttccctagctttagaccctgggtgggctctgtgcagtcactggaggttgaagccaagtggggtgctgggaggagggagagggaggtcactggaaaggggagagcctgctggcacccaccgtggaggaggaaggcaagagggggtggaggggtgtggcagtggttttggcaaacgctaaagagcccttgcctccccatttcccatctgcaccccttctctcctccccaaatcaatacactagttgtttctacccctggcaaaaaaaaaaaa

SEQ ID NO: 4 - DNA, 3181 bp, Homo sapiens
actcagtgttcgcgggagcgccgcacctacaccagccaacccagatcccgaggtccgacagcgcccggcccagatccccacgcctgccaggagcaagccgagagccagccggccggcgcactccgactccgagcagtctctgtccttcgacccgagccccgcgccctttccgggacccctgccccgcgggcagcgctgccaacctgccggccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacggctcccactgcagcagctcgggggaccccgctgagtacaacctgcgctcgcgcaccgtgctgtgcgggacctgcgggcagcctgccgacaaggcatctgccagcggctcaggagcccaggtgggtggacccatctcctctggctcttctgcctccagtgtcacggtcactcgcagctaccgcagtgtggggggcagtgggggtggcagcttcggggacaatctggtcacccgctcctacctcctgggcaactccagcccccgaacccagagcccccagaactgcagcatcatgtaatctgggacctgccaggcaggggtgggggtggaggcttcctgcgtcctcctcacctcatgcccaccccctgccctgcacgtcatgggagggggcttgaagccaaagaaaaataaccctttggtttttttcttctgtatttttttttctaagagaagttattttctacagtggttttatactgaaggaaaaacacaagcaaaaaaaaaaaaaagcatctatctcatctatctcaatcctaatttctcctcccttccttttccctgcttccaggaaactccacatctgccttaaaaccaaagagggcttcctctagaagccaagggaaaggggtgcttttatagaggctagcttctgcttttctgccctggctgctgcccccaccccggggaccctgtgacatggtgcctgagaggcaggcatagaggcttctccgccagcctcctctggacggcaggctcactgccaggccagcctccgagagggagagagagagagagaggacagcttgagccgggcccctgggcttggcctgctgtgattccactacacctggctgaggttcctctgcctgccccgcccccagtccccacccctgcccccagccccggggtgagtccattctcccaggtaccagctgcgcttgcttttctgtattttatttagacaagagatgggaatgaggtgggaggtggaagaagggagaagaaaggtgagtttgagctgccttccctagctttagaccctgggtgggctctgtgcagtcactggaggttgaagccaagtggggtgctgggaggagggagagggaggtcactggaaaggggagagcctgctggcacccaccgtggaggaggaaggcaagagggggtggaggggtgtggcagtggttttggcaaacgctaaagagcccttgcctccccatttcccatctgcaccccttctctcctccccaaatcaatacactagttgtttct

SEQ ID NO: 5 - DNA, 19 bp, Artificial sequence
  Primer
cagcttcggggacaatctg

SEQ ID NO: 6 - DNA, 19 bp, Artificial sequence
  Primer
ggcatgaggtgaggaggac

SEQ ID NO: 7 - DNA, 20 bp, Artificial sequence
  Probe
gtcacccgctcctacctcct

SEQ ID NO: 8 - DNA, 18 bp, Artificial sequence
  Primer
gcgtcaggagccctgagc

SEQ ID NO: 9 - DNA, 18 bp, Artificial sequence
  Primer
gacgcaggaagcctccac

SEQ ID NO: 10 - DNA, 21 bp, Artificial sequence
  Probe
agcatcatgtaatctgggacc

SEQ ID NO: 11 - DNA, 20 bp, Artificial sequence
  Primer
acggctctcatcaactccac

SEQ ID NO: 12 - DNA, 18 bp, Artificial sequence
  Primer
gcggcggctaccactcac

SEQ ID NO: 13 - DNA, 20 bp, Artificial sequence
  Probe
ggttgaggacgacgaggatg

SEQ ID NO: 14 - DNA, 18 bp, Artificial sequence
  Synthetic oligonucleotide
agcacggtgcgcgagcgc

SEQ ID NO: 15 - DNA, 18 bp, Artificial sequence
  Synthetic oligonucleotide
cgcacagcacggtgcgcg

SEQ ID NO: 16 - DNA, 18 bp, Artificial sequence
  Synthetic oligonucleotide
ggtcccgcacagcacggt

SEQ ID NO: 17 - DNA, 18 bp, Artificial sequence
  Synthetic oligonucleotide
ccgcaggtcccgcacagc

SEQ ID NO: 18 - DNA, 18 bp, Artificial sequence
  Synthetic oligonucleotide
gctgcccgcaggtcccgc

SEQ ID NO: 19 - DNA, 18 bp, Artificial sequence
  Synthetic oligonucleotide
ggcaggctgcccgcaggt

SEQ ID NO: 20 - DNA, 18 bp, Artificial sequence
  Synthetic oligonucleotide
ttgtcggcaggctgcccg

SEQ ID NO: 21 - DNA, 18 bp, Artificial sequence
  Synthetic oligonucleotide
atgccttgtcggcaggct

SEQ ID NO: 22 - DNA, 18 bp, Artificial sequence
  Synthetic oligonucleotide
ggcagatgccttgtcggc

SEQ ID NO: 23 - DNA, 18 bp, Artificial sequence
  Synthetic oligonucleotide
ccgctggcagatgccttg

SEQ ID NO: 24 - DNA, 18 bp, Artificial sequence
  Synthetic oligonucleotide
ctgagccgctggcagatg

SEQ ID NO: 25 - DNA, 18 bp, Artificial sequence
  Synthetic oligonucleotide
ggctcctgagccgctggc

SEQ ID NO: 26 - DNA, 18 bp, Artificial sequence
  Synthetic oligonucleotide
tgggctcctgagccgctg

SEQ ID NO: 27 - DNA, 18 bp, Artificial sequence
  Synthetic oligonucleotide
cctgggctcctgagccgc

SEQ ID NO: 28 - DNA, 18 bp, Artificial sequence
  Synthetic oligonucleotide
cacctgggctcctgagcc

SEQ ID NO: 29 - DNA, 18 bp, Artificial sequence
  Synthetic oligonucleotide
cccacctgggctcctgag

SEQ ID NO: 30 - DNA, 18 bp, Artificial sequence
  Synthetic oligonucleotide
cacccacctgggctcctg

SEQ ID NO: 31 - DNA, 18 bp, Artificial sequence
  Synthetic oligonucleotide
tccacccacctgggctcc

SEQ ID NO: 32 - DNA, 18 bp, Artificial sequence
  Synthetic oligonucleotide
ggtccacccacctgggct

SEQ ID NO: 33 - DNA, 18 bp, Artificial sequence
  Synthetic oligonucleotide
tgggtccacccacctggg

SEQ ID NO: 34 - DNA, 18 bp, Artificial sequence
  Synthetic oligonucleotide
gatgggtccacccacctg

SEQ ID NO: 35 - DNA, 18 bp, Artificial sequence
  Synthetic oligonucleotide
gagatgggtccacccacc

SEQ ID NO: 36 - DNA, 18 bp, Artificial sequence
  Synthetic oligonucleotide
aggagatgggtccaccca

SEQ ID NO: 37 - DNA, 18 bp, Artificial sequence
  Synthetic oligonucleotide
agaggagatgggtccacc

SEQ ID NO: 38 - DNA, 18 bp, Artificial sequence
  Synthetic oligonucleotide
ccagaggagatgggtcca

SEQ ID NO: 39 - DNA, 16 bp, Artificial sequence
  Synthetic oligonucleotide
cccacctgggctcctg

SEQ ID NO: 40 - DNA, 16 bp, Artificial sequence
  Synthetic oligonucleotide
cacccacctgggctcc

SEQ ID NO: 41 - DNA, 16 bp, Artificial sequence
  Synthetic oligonucleotide
tccacccacctgggct

SEQ ID NO: 42 - DNA, 16 bp, Artificial sequence
  Synthetic oligonucleotide
ggtccacccacctggg

SEQ ID NO: 43 - DNA, 16 bp, Artificial sequence
  Synthetic oligonucleotide
tgggtccacccacctg

SEQ ID NO: 44 - DNA, 18 bp, Artificial sequence
  Synthetic oligonucleotide
gtcctcaaccacagtcac

SEQ ID NO: 45 - DNA, 18 bp, Artificial sequence
  Synthetic oligonucleotide
tcgtcgtcctcaaccaca

SEQ ID NO: 46 - DNA, 18 bp, Artificial sequence
  Synthetic oligonucleotide
catcctcgtcgtcctcaa

SEQ ID NO: 47 - DNA, 18 bp, Artificial sequence
  Synthetic oligonucleotide
atcctcatcctcgtcgtc

SEQ ID NO: 48 - DNA, 18 bp, Artificial sequence
  Synthetic oligonucleotide
tctccatcctcatcctcg

SEQ ID NO: 49 - DNA, 18 bp, Artificial sequence
  Synthetic oligonucleotide
ggtcatctccatcctcat

SEQ ID NO: 50 - DNA, 18 bp, Artificial sequence
  Synthetic oligonucleotide
gagcaggtcatctccatc

SEQ ID NO: 51 - DNA, 18 bp, Artificial sequence
  Synthetic oligonucleotide
tgatggagcaggtcatct

SEQ ID NO: 52 - DNA, 18 bp, Artificial sequence
  Synthetic oligonucleotide
ggtggtgatggagcaggt

SEQ ID NO: 53 - DNA, 18 bp, Artificial sequence
  Synthetic oligonucleotide
gtggtggtgatggagcag

SEQ ID NO: 54 - DNA, 18 bp, Artificial sequence
  Synthetic oligonucleotide
acgtggtggtgatggagc

SEQ ID NO: 55 - DNA, 18 bp, Artificial sequence
  Synthetic oligonucleotide
tcacgtggtggtgatgga

SEQ ID NO: 56 - DNA, 18 bp, Artificial sequence
  Synthetic oligonucleotide
actcacgtggtggtgatg

SEQ ID NO: 57 - DNA, 18 bp, Artificial sequence
  Synthetic oligonucleotide
ccactcacgtggtggtga

SEQ ID NO: 58 - DNA, 18 bp, Artificial sequence
  Synthetic oligonucleotide
taccactcacgtggtggt

SEQ ID NO: 59 - DNA, 18 bp, Artificial sequence
  Synthetic oligonucleotide
gctaccactcacgtggtg

SEQ ID NO: 60 - DNA, 18 bp, Artificial sequence
  Synthetic oligonucleotide
cggctaccactcacgtgg

SEQ ID NO: 61 - DNA, 18 bp, Artificial sequence
  Synthetic oligonucleotide
ggcggctaccactcacgt

SEQ ID NO: 62 - DNA, 18 bp, Artificial sequence
  Synthetic oligonucleotide
gcggcggctaccactcac

SEQ ID NO: 63 - DNA, 18 bp, Artificial sequence
  Synthetic oligonucleotide
cagcggcggctaccactc

SEQ ID NO: 64 - DNA, 18 bp, Artificial sequence
  Synthetic oligonucleotide
ctcagcggcggctaccac

SEQ ID NO: 65 - DNA, 18 bp, Artificial sequence
  Synthetic oligonucleotide
gcctcagcggcggctacc

SEQ ID NO: 66 - DNA, 18 bp, Artificial sequence
  Synthetic oligonucleotide
gctcggcctcagcggcgg

SEQ ID NO: 67 - DNA, 18 bp, Artificial sequence
  Synthetic oligonucleotide
tgcaggctcggcctcagc

SEQ ID NO: 68 - DNA, 18 bp, Artificial sequence
  Synthetic oligonucleotide
cccagtgcaggctcggcc

SEQ ID NO: 69 - DNA, 18 bp, Artificial sequence
  Synthetic oligonucleotide
gtggccccagtgcaggct

SEQ ID NO: 70 - DNA, 18 bp, Artificial sequence
  Synthetic oligonucleotide
gctgggtggccccagtgc

SEQ ID NO: 71 - DNA, 18 bp, Artificial sequence
  Synthetic oligonucleotide
gcctggctgggtggcccc

SEQ ID NO: 72 - DNA, 18 bp, Artificial sequence
  Synthetic oligonucleotide
cccaggcctggctgggtg

SEQ ID NO: 73 - DNA, 18 bp, Artificial sequence
  Synthetic oligonucleotide
ctgcccccaggcctggct

SEQ ID NO: 74 - DNA, 25 bp, Artificial sequence
  Synthetic oligonucleotide
gggtccacccacctgggctcctgag

SEQ ID NO: 75 - DNA, 25 bp, Artificial sequence
  Synthetic oligonucleotide
ctgggctcctgagccgctggcagat

SEQ ID NO: 76 - DNA, 25 bp, Artificial sequence
  Synthetic oligonucleotide
acctgggctcctgagccgctggcag

SEQ ID NO: 77 - DNA, 25 bp, Artificial sequence
  Synthetic oligonucleotide
ccacctgggctcctgagccgctggc

SEQ ID NO: 78 - DNA, 25 bp, Artificial sequence
  Synthetic oligonucleotide
acccacctgggctcctgagccgctg

SEQ ID NO: 79 - DNA, 25 bp, Artificial sequence
  Synthetic oligonucleotide
ccacccacctgggctcctgagccgc

SEQ ID NO: 80 - DNA, 25 bp, Artificial sequence
  Synthetic oligonucleotide
gtccacccacctgggctcctgagcc

SEQ ID NO: 81 - DNA, 25 bp, Artificial sequence
  Synthetic oligonucleotide
atgggtccacccacctgggctcctg

SEQ ID NO: 82 - DNA, 25 bp, Artificial sequence
  Synthetic oligonucleotide
agatgggtccacccacctgggctcc

SEQ ID NO: 83 - DNA, 25 bp, Artificial sequence
  Synthetic oligonucleotide
ggagatgggtccacccacctgggct

SEQ ID NO: 84 - DNA, 25 bp, Artificial sequence
  Synthetic oligonucleotide
gaggagatgggtccacccacctggg

SEQ ID NO: 85 - DNA, 25 bp, Artificial sequence
  Synthetic oligonucleotide
cagaggagatgggtccacccacctg

SEQ ID NO: 86 - DNA, 25 bp, Artificial sequence
  Synthetic oligonucleotide
gccagaggagatgggtccacccacc

SEQ ID NO: 87 - DNA, 25 bp, Artificial sequence
  Synthetic oligonucleotide
gagccagaggagatgggtccaccca

SEQ ID NO: 88 - DNA, 25 bp, Artificial sequence
  Synthetic oligonucleotide
aagagccagaggagatgggtccacc

SEQ ID NO: 89 - DNA, 25 bp, Artificial sequence
  Synthetic oligonucleotide
agaagagccagaggagatgggtcca

SEQ ID NO: 90 - DNA, 25 bp, Artificial sequence
  Synthetic oligonucleotide
gcagaagagccagaggagatgggtc

SEQ ID NO: 91 - DNA, 20 bp, Artificial sequence
  Synthetic oligonucleotide
ggggctctgggctcctgagc

SEQ ID NO: 92 - DNA, 20 bp, Artificial sequence
  Synthetic oligonucleotide
gggggctctgggctcctgag

SEQ ID NO: 93 - DNA, 20 bp, Artificial sequence
  Synthetic oligonucleotide
tgggggctctgggctcctga

SEQ ID NO: 94 - DNA, 20 bp, Artificial sequence
  Synthetic oligonucleotide
agttctgggggctctgggct

SEQ ID NO: 95 - DNA, 20 bp, Artificial sequence
  Synthetic oligonucleotide
cagttctgggggctctgggc

SEQ ID NO: 96 - DNA, 20 bp, Artificial sequence
  Synthetic oligonucleotide
gcagttctgggggctctggg

SEQ ID NO: 97 - DNA, 20 bp, Artificial sequence
  Synthetic oligonucleotide
gctgcagttctgggggctct

SEQ ID NO: 98 - DNA, 20 bp, Artificial sequence
  Synthetic oligonucleotide
tgctgcagttctgggggctc

SEQ ID NO: 99 - DNA, 20 bp, Artificial sequence
  Synthetic oligonucleotide
ctgggctcctgagccgctgg

SEQ ID NO: 100 - DNA, 20 bp, Artificial sequence
  Synthetic oligonucleotide
ctctgggctcctgagccgct

SEQ ID NO: 101 - DNA, 20 bp, Artificial sequence
  Synthetic oligonucleotide
gctctgggctcctgagccgc

SEQ ID NO: 102 - DNA, 20 bp, Artificial sequence
  Synthetic oligonucleotide
tctgggggctctgggctcct

SEQ ID NO: 103 - DNA, 20 bp, Artificial sequence
  Synthetic oligonucleotide
gggctctgggctcctgagcc

SEQ ID NO: 104 - DNA, 20 bp, Artificial sequence
  Synthetic oligonucleotide
ctgcagttctgggggctctg

SEQ ID NO: 105 - DNA, 20 bp, Artificial sequence
  Synthetic oligonucleotide
tctgggctcctgagccgctg

SEQ ID NO: 106 - DNA, 20 bp, Artificial sequence
  Synthetic oligonucleotide
ggctctgggctcctgagccg

SEQ ID NO: 107 - DNA, 20 bp, Artificial sequence
  Synthetic oligonucleotide
ctgggggctctgggctcctg

SEQ ID NO: 108 - DNA, 20 bp, Artificial sequence
  Synthetic oligonucleotide
ttctgggggctctgggctcc

SEQ ID NO: 109 - DNA, 20 bp, Artificial sequence
  Synthetic oligonucleotide
gttctgggggctctgggctc

SEQ ID NO: 110 - DNA, 20 bp, Artificial sequence
  Synthetic oligonucleotide
tgcagttctgggggctctgg

SEQ ID NO: 111 - DNA, 20 bp, Artificial sequence
  Synthetic oligonucleotide
atgctgcagttctgggggct

SEQ ID NO: 112 - DNA, 16 bp, Artificial sequence
  Synthetic oligonucleotide
ctgggctcctgagccg

SEQ ID NO: 113 - DNA, 16 bp, Artificial sequence
  Synthetic oligonucleotide
tctgggctcctgagcc

SEQ ID NO: 114 - DNA, 16 bp, Artificial sequence
  Synthetic oligonucleotide
ctctgggctcctgagc

SEQ ID NO: 115 - DNA, 16 bp, Artificial sequence
  Synthetic oligonucleotide
gctctgggctcctgag

SEQ ID NO: 116 - DNA, 16 bp, Artificial sequence
  Synthetic oligonucleotide
ggctctgggctcctga

SEQ ID NO: 117 - DNA, 16 bp, Artificial sequence
  Synthetic oligonucleotide
gggctctgggctcctg

SEQ ID NO: 118 - DNA, 16 bp, Artificial sequence
  Synthetic oligonucleotide
ggggctctgggctcct

SEQ ID NO: 119 - DNA, 16 bp, Artificial sequence
  Synthetic oligonucleotide
gggggctctgggctcc

SEQ ID NO: 120 - DNA, 16 bp, Artificial sequence
  Synthetic oligonucleotide
tgggggctctgggctc

SEQ ID NO: 121 - DNA, 16 bp, Artificial sequence
  Synthetic oligonucleotide
ctgggggctctgggct

SEQ ID NO: 122 - DNA, 16 bp, Artificial sequence
  Synthetic oligonucleotide
tctgggggctctgggc

SEQ ID NO: 123 - DNA, 16 bp, Artificial sequence
  Synthetic oligonucleotide
ttctgggggctctggg

SEQ ID NO: 124 - DNA, 16 bp, Artificial sequence
  Synthetic oligonucleotide
gttctgggggctctgg

SEQ ID NO: 125 - DNA, 16 bp, Artificial sequence
  Synthetic oligonucleotide
agttctgggggctctg

SEQ ID NO: 126 - DNA, 16 bp, Artificial sequence
  Synthetic oligonucleotide
cagttctgggggctct

SEQ ID NO: 127 - DNA, 16 bp, Artificial sequence
  Synthetic oligonucleotide
gcagttctgggggctc

SEQ ID NO: 128 - DNA, 16 bp, Artificial sequence
  Synthetic oligonucleotide
tgcagttctgggggct

SEQ ID NO: 129 - DNA, 16 bp, Artificial sequence
  Synthetic oligonucleotide
atgggtccacccacct

SEQ ID NO: 130 - DNA, 16 bp, Artificial sequence
  Synthetic oligonucleotide
gagatgggtccaccca

SEQ ID NO: 131 - DNA, 16 bp, Artificial sequence
  Synthetic oligonucleotide
ggagatgggtccaccc

SEQ ID NO: 132 - DNA, 20 bp, Artificial sequence
  Synthetic oligonucleotide
gggctctaagagagaaaaca

SEQ ID NO: 133 - DNA, 16 bp, Artificial sequence
  Synthetic oligonucleotide
gctctaagagagaaaa

SEQ ID NO: 134 - DNA, 16 bp, Artificial sequence
  Synthetic oligonucleotide
ggctctaagagagaaa

SEQ ID NO: 135 - DNA, 16 bp, Artificial sequence
  Synthetic oligonucleotide
gggctctaagagagaa

SEQ ID NO: 136 - DNA, 15 bp, Artificial sequence
  Synthetic oligonucleotide
gggctctaagagaga

SEQ ID NO: 137 - DNA, 14 bp, Artificial sequence
  Synthetic oligonucleotide
gggctctaagagag

SEQ ID NO: 138 - DNA, 14 bp, Artificial sequence
  Synthetic oligonucleotide
tgggctctaagaga

SEQ ID NO: 139 - DNA, 14 bp, Artificial sequence
  Synthetic oligonucleotide
ctgggctctaagag

SEQ ID NO: 140 - DNA, 14 bp, Artificial sequence
  Synthetic oligonucleotide
tctgggctctaaga

SEQ ID NO: 141 - DNA, 14 bp, Artificial sequence
  Synthetic oligonucleotide
ttctgggctctaag

SEQ ID NO: 142 - DNA, 14 bp, Artificial sequence
  Synthetic oligonucleotide
gttctgggctctaa

SEQ ID NO: 143 - DNA, 14 bp, Artificial sequence
  Synthetic oligonucleotide
cagttctgggctct

SEQ ID NO: 144 - DNA, 18 bp, Artificial sequence
  Synthetic oligonucleotide
gcagttctgggctctaag

SEQ ID NO: 145 - DNA, 18 bp, Artificial sequence
  Synthetic oligonucleotide
ctgcagttctgggctcta

SEQ ID NO: 146 - DNA, 18 bp, Artificial sequence
  Synthetic oligonucleotide
tgggctctaagagagaaa

SEQ ID NO: 147 - DNA, 18 bp, Artificial sequence
  Synthetic oligonucleotide
ctgggctctaagagagaa

SEQ ID NO: 148 - DNA, 18 bp, Artificial sequence
  Synthetic oligonucleotide
tctgggctctaagagaga

SEQ ID NO: 149 - DNA, 18 bp, Artificial sequence
  Synthetic oligonucleotide
ttctgggctctaagagag

SEQ ID NO: 150 - DNA, 18 bp, Artificial sequence
  Synthetic oligonucleotide
tgcagttctgggctctaa

SEQ ID NO: 151 - DNA, 18 bp, Artificial sequence
  Synthetic oligonucleotide
gctgcagttctgggctct

SEQ ID NO: 152 - DNA, 18 bp, Artificial sequence
  Synthetic oligonucleotide
gttctgggctctaagaga

SEQ ID NO: 153 - DNA, 18 bp, Artificial sequence
  Synthetic oligonucleotide
agttctgggctctaagag

SEQ ID NO: 154 - DNA, 20 bp, Artificial sequence
  Synthetic oligonucleotide
agatgggtccacccacctgg

SEQ ID NO: 155 - DNA, 20 bp, Artificial sequence
  Synthetic oligonucleotide
gagatgggtccacccacctg

SEQ ID NO: 156 - DNA, 20 bp, Artificial sequence
  Synthetic oligonucleotide
aggagatgggtccacccacc

SEQ ID NO: 157 - DNA, 20 bp, Artificial sequence
  Synthetic oligonucleotide
gccagaggagatgggtccac

SEQ ID NO: 158 - DNA, 2501 bp, Homo sapiens
gtagtttcccgcccttgggggcgcggggacaaattccttgacccgaggaggatagggatgtggccttcggtctttcctcgcagctccggggcaagctaggagtgggatggaagtcgagagtcgatcccggagcccggccgcggggagaggttctcggcagagaagacaaagcccgcagcagcgatggggggagagctgggctctgcgtgttgtgggggccaggaaagggtgccaggctggggctggaaccccctggcaaaggatggggtcccctcatccctaaacagcaagccatctcccctcgcccgccccccgcccccccagtctcggagatctcagaggcaccgactgggagacttgatgtaattgtcttcacaattctgtgaagatggacttggtgtctctgcttcacatgaagggacctgaggttcagagaggctaagtaactttcctaggtcacagagctgtgttcgcgggagcgccgcacctacaccagccaacccagatcccgaggtccgacagcgcccggcccagatccccacgcctgccaggagcaagccgagagccagccggccggcgcactccgactccgagcagtctctgtccttcgacccgagccccgcgccctttccgggacccctgccccgcgggcagcgctgccaacctgccggccatggagaccccgtcccagcggcgcgccacccgcagcggggcgcaggccagctccactccgctgtcgcccacccgcatcacccggctgcaggagaaggaggacctgcaggagctcaatgatcgcttggcggtctacatcgaccgtgtgcgctcgctggaaacggagaacgcagggctgcgccttcgcatcaccgagtctgaagaggtggtcagccgcgaggtgtccggcatcaaggccgcctacgaggccgagctcggggatgcccgcaagacccttgactcagtagccaaggagcgcgcccgcctgcagctggagctgagcaaagtgcgtgaggagtttaaggagctgaaagcgcgcaataccaagaaggagggtgacctgatagctgctcaggctcggctgaaggacctggaggctctgctgaactccaaggaggccgcactgagcactgctctcagtgagaagcgcacgctggagggcgagctgcatgatctgcggggccaggtggccaagcttgaggcagccctaggtgaggccaagaagcaacttcaggatgagatgctgcggcgggtggatgctgagaacaggctgcagaccatgaaggaggaactggacttccagaagaacatctacagtgaggagctgcgtgagaccaagcgccgtcatgagacccgactggtggagattgacaatgggaagcagcgtgagtttgagagccggctggcggatgcgctgcaggaactgcgggcccagcatgaggaccaggtggagcagtataagaaggagctggagaagacttattctgccaagctggacaatgccaggcagtctgctgagaggaacagcaacctggtgggggctgcccacgaggagctgcagcagtcgcgcatccgcatcgacagcctctctgcccagctcagccagctccagaagcagctggcagccaaggaggcgaagcttcgagacctggaggactcactggcccgtgagcgggacaccagccggcggctgctggcggaaaaggagcgggagatggccgagatgcgggcaaggatgcagcagcagctggacgagtaccaggagcttctggacatcaagctggccctggacatggagatccacgcctaccgcaagctcttggagggcgaggaggagaggctacgcctgtcccccagccctacctcgcagcgcagccgtggccgtgcttcctctcactcatcccagacacagggtgggggcagcgtcaccaaaaagcgcaaactggagtccactgagagccgcagcagcttctcacagcacgcacgcactagcgggcgcgtggccgtggaggaggtggatgaggagggcaagtttgtccggctgcgcaacaagtccaatgaggaccagtccatgggcaattggcagatcaagcgccagaatggagatgatcccttgctgacttaccggttcccaccaaagttcaccctgaaggctgggcaggtggtgacgatctgggctgcaggagctggggccacccacagcccccctaccgacctggtgtggaaggcacagaacacctggggctgcgggaacagcctgcgtacggctctcatcaactccactggggaagaagtggccatgcgcaagctggtgcgctcagtgactgtggttgaggacgacgaggatgaggatggagatgacctgctccatcaccaccacgtgagtggtagccgccgctgaggccgagcctgcactggggccacccagccaggcctgggggcagcctctccccagcctccccgtgccaaaaatcttttcattaaagaatgttttggaactttaaaaaaaa

SEQ ID NO: 159 - DNA, 20 bp, Artificial sequence
  synthetic oligonucleotide
gctcatttagtctgcctgat

(19) United States
(12) Patent Application Publication
(10) Pub. No.: US 2022/0031731 A1
(43) Pub. Date: Feb. 3, 2022

(54) COMPOSITIONS AND METHODS FOR MODULATION OF LMNA EXPRESSION

(71) Applicants: Ionis Pharmaceuticals, Inc., Carlsbad, CA (US); THE UNITED STATES OF AMERICA , AS REPRESENTED BY THE SECRETARY , DEPARTMENT OF HEALTH AND HUMAN SERV, Rockville, CA (US)

(72) Inventors: Priyam Singh, San Diego, CA (US); Frank Rigo, Carlsbad, CA (US); Tom Misteli, Bethesda, MD (US); Madaiah Puttaraju, Bethesda, MD (US)

(73) Assignees: Ionis Pharmaceuticals, Inc., Carlsbad, CA (US); Ionis Pharmaceuticals, Inc., Rockville, MD (US)

(21) Appl. No.: 17/277,397

(22) PCT Filed: Sep. 20, 2019
(86) PCT No.: PCT/US2019/052223

§ 371 Date: Mar. 18, 2021

Related U.S. Application Data
(60) Provisional application No. 62/734,196, filed on Sep. 20, 2018.

(51) Int. Cl. A61K 31/713; C12N 15/113; A61P 43/00

(52) U.S. Cl. CPC A61K 31/713; A61P 43/00; C12N 15/113

(57) ABSTRACT

The present disclosure provides compounds comprising oligonucleotides complementary to a portion of the LMNA gene. Such compounds are useful for modulating the expression of LMNA in a cell or animal, and in certain instances reducing the amount of progerin mRNA and/or progerin protein. Progerin mRNA results from aberrant splicing of LMNA and is translated to generate progerin protein. Accumulation of progerin protein causes Hutchinson-Gilford progeria syndrome (HOPS), a premature aging disease. In certain embodiments, hybridization of oligonucleotides complementary to a portion of the LMNA gene results in a decrease in the amount of progerin mRNA and/or progerin protein. In certain embodiments, oligonucleotides are used to treat Hutchinson-Gilford Progeria Syndrome.

### STATEMENT OF GOVERNMENT SUPPORT

This work was supported by the Intramural Research Program of NIH, NCI grant 1ZIA BC01030919 The United States government has rights in the inventive subject matter by virtue of this support.

### SEQUENCE LISTING

The present application is being filed along with a Sequence Listing in electronic format. The Sequence Listing is provided as a file entitled BIOL0342WOSEQ_ST25.txt, created on Sep. 17, 2019, which is 76 KB in size. The information in the electronic format of the sequence listing is incorporated herein by reference in its entirety.

### FIELD

Provided are compounds, methods, and pharmaceutical compositions for modulating the expression of LMNA pre-mRNA or mRNA in a cell or animal, and in certain instances modulating the amount or type of protein in a cell or animal. Such compounds, methods, and pharmaceutical compositions are useful to ameliorate at least one symptom of Hutchinson-Gilford progeria syndrome (HGPS). Such symptoms include a lack of subcutaneous fat, sclerotic skin, joint contractures, bone abnormalities, weight loss, hair loss, hypertension, metabolic syndrome, central nervous system sequelae, conductive hearing loss, oral deficits, craniofacial abnormalities, progressive cardiovascular disease resembling atherosclerosis, congestive heart failure, and premature death.

### BACKGROUND

The LMNA gene encodes several alternatively spliced products, including prelamin A mRNA, progerin mRNA, and lamin C mRNA (Vidak and Foisner, Histochem Cell Biology, 2016). The primary protein products are lamin A and lamin C. Production of progerin mRNA and progerin protein occurs rarely in healthy cells. The N-terminal 566 amino acids of lamin A and lamin C are identical with lamin C containing 6 unique amino acids at the C-terminus to yield a protein of 572 amino acids. Lamin A, which is 646 amino acids in length, is generated from a precursor protein, prelamin A, by a series of posttranslational processing steps (Young et al., 2005, J. Lipid Res. October 5 electronic publication). The first step in prelamin A processing is farnesylation of a carboxyl-terminal cysteine residue, which is part of a CAAX motif at the terminus of the protein. Next, the terminal three amino acids (AAX) are cleaved from the protein, after which the farnesylcysteine is methylated. Finally, the C-terminal 15 amino acids are enzymatically removed and degraded to form mature lamin A.

Lamin A and lamin C are key structural components of the nuclear lamina, an intermediate filament meshwork underneath the inner nuclear membrane. The lamin proteins comprise N-terminal globular head domains, central helical rod domains and C-terminal globular tail domains. Lamins A and C homodimerize to form parallel coiled-coil dimers, which associate head-to-tail to form strings, and ultimately form the higher-order filamentous meshwork that provides structural support for the nucleus (Muchir and Worman, 2004, Physiology (Bethesda) 19: 309-314; Mutchison and Worman, 2004, Nat. Cell Biol. 6: 1062-1067; Mounkes et al. 2001, Trends Cardiovasc. Med. 11: 280-285).

Hutchinson-Gilford progeria syndrome (HGPS) is a childhood premature aging disease resulting from the production of a mutant form of farnesyl-prelamin A, progerin protein, which cannot be processed to mature lamin A. The accumulation of the farnesylated progerin protein is toxic, inducing misshapen nuclei and aberrant regulation of gene expression at the cellular level and a wide range of disease symptoms at the organismal level (e.g., a lack of subcutaneous fat, sclerotic skin, joint contractures, bone abnormalities, weight loss, hair loss, hypertension, metabolic syndrome, central nervous system sequelae, conductive hearing loss, oral deficits, craniofacial abnormalities, progressive cardiovascular disease resembling atherosclerosis, congestive heart failure, and premature death). HGPS is most commonly caused by a spontaneous mutation in exon 11 of LMNA, which activates a cryptic splice site four nucleotides upstream of the mutation (a cytosine to thymidine substitution at codon 608, also known as a G608G mutation) (Eriksson et al. 2003, Nature 423: 293-298). The pre-mRNA derived from the mutated allele is spliced using the aberrant donor splice site and the correct exon 12 acceptor site, yielding a truncated LMNA mRNA lacking the terminal 150 nucleotides of exon 11. This truncated mRNA lacking a portion of exon 11 is known as progerin mRNA. As a result of this aberrant splicing, a mutant protein lacking 50 amino acids from the globular tail is produced. This shortened version of prelamin A is known as progerin protein. Like prelamin A, progerin is farnesylated. Unlike prelamin A, progerin does not undergo further maturation, and instead the farnesylated progerin accumulates.

Currently there are a lack of acceptable options for treating HGPS. It is therefore an object herein to provide compounds, methods, and pharmaceutical compositions for the treatment of HGPS.

### SUMMARY OF THE INVENTION

Provided are oligomeric compounds, methods, and pharmaceutical compositions for modulating the expression of LMNA in a cell or animal, and in certain instances reducing the amount of progerin mRNA and/or progerin protein. Progerin mRNA results from aberrant splicing of LMNA and is translated to generate progerin protein. Accumulation of progerin protein causes Hutchinson-Gilford progeria syndrome (HGPS), a premature aging disease.

In certain embodiments, oligomeric compounds or modified oligonucleotides described herein modulate the splicing of LMNA. In certain embodiments, oligomeric compounds or modified oligonucleotides described herein reduce progerin mRNA and increase lamin C mRNA. In certain embodiments, oligomeric compounds or modified oligonucleotides recruit RNAse H to degrade LMNA pre-mRNA or LMNA mRNA, including prelamin A mRNA and progerin mRNA.

Also provided are methods useful for ameliorating at least one symptom of a premature aging disease. In certain embodiments, the premature aging disease is Hutchinson-Gilford progeria syndrome (HGPS). In certain embodiments, symptoms include misshapen nuclei and aberrant regulation of gene expression at the cellular level. In certain embodiments, symptoms include a lack of subcutaneous fat, sclerotic skin, joint contractures, bone abnormalities, weight loss, hair loss, hypertension, metabolic syndrome, central nervous system sequelae, conductive hearing loss, oral deficits, craniofacial abnormalities, progressive cardiovascular disease resembling atherosclerosis, congestive heart failure, and premature death. In certain embodiments, amelioration of these symptoms results in a reduction in weight loss. In certain embodiments, amelioration of these symptoms results in prolonged survival.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, and treatises, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout in the disclosure are incorporated by reference herein in their entirety.

Unless otherwise indicated, the following terms have the following meanings:

### Definitions

As used herein, “2′-deoxyribonucleoside” means a nucleoside comprising a 2′-H(H) deoxyribosyl sugar moiety, as found in naturally occurring deoxyribonucleic acids (DNA). In certain embodiments, a 2′-deoxyribonucleoside may comprise a modified nucleobase or may comprise an RNA nucleobase (uracil). In certain embodiments, a 2′-deoxyribonucleoside may comprise hypoxanthine. In certain embodiments, a 2′-deoxyribonucleoside is in the β-D configuration, and is referred to as a nucleoside comprising a β-D-2′-deoxyribose sugar moiety.

As used herein, “2′-substituted nucleoside” means a nucleoside comprising a 2′-substituted sugar moiety. As used herein, “2′-substituted” in reference to a sugar moiety means a sugar moiety comprising at least one 2′-substituent group other than H or OH.

As used herein, “5-methyl cytosine” means a cytosine modified with a methyl group attached to the 5-position. A 5-methyl cytosine is a modified nucleobase.

As used herein, “administering” means providing a pharmaceutical agent to an animal.

As used herein, “administered concomitantly” or “co-administration” means administration of two or more compounds in any manner in which the pharmacological effects of both are manifest in the patient. Concomitant administration does not require that both compounds be administered in a single pharmaceutical composition, in the same dosage form, by the same route of administration, or at the same time. The effects of both compounds need not manifest themselves at the same time. The effects need only be overlapping for a period of time and need not be coextensive. Concomitant administration or co-administration encompasses administration in parallel, sequentially, separate, or simultaneous administration.

As used herein, “animal” means a human or non-human animal.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense compound” means an oligomeric compound or oligomeric duplex capable of achieving at least one antisense activity.

As used herein, “ameliorate” in reference to a treatment means improvement in at least one symptom relative to the same symptom in the absence of the treatment. In certain embodiments, amelioration is the reduction in the severity or frequency of a symptom or the delayed onset or slowing of progression in the severity or frequency of a symptom. In certain embodiments, the symptom is a lack of subcutaneous fat, weight loss, hair loss, hypertension, metabolic syndrome, progressive cardiovascular disease resembling atherosclerosis, congestive heart failure, or premature death. In certain embodiments, amelioration of these symptoms results in a reduction of weight loss and increased survival.

As used herein, “bicyclic nucleoside” or “BNA” means a nucleoside comprising a bicyclic sugar moiety. As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl moiety. In certain embodiments, the bicyclic sugar moiety does not comprise a furanosyl moiety.

As used herein, “cleavable moiety” means a bond or group of atoms that is cleaved under physiological conditions, for example, inside a cell, an animal, or a human.

As used herein, “complementary” in reference to an oligonucleotide means that at least 70% of the nucleobases of the oligonucleotide or one or more regions thereof and the nucleobases of another nucleic acid or one or more regions thereof are capable of hydrogen bonding with one another when the nucleobase sequence of the oligonucleotide and the other nucleic acid are aligned in opposing directions. Complementary nucleobases means nucleobases that are capable of forming hydrogen bonds with one another. Complementary nucleobase pairs include adenine (A) and thymine (T), adenine (A) and uracil (U), cytosine (C) and guanine (G), 5-methyl cytosine (mC) and guanine (G). The nucleobase hypoxanthine (I) is able to hydrogen bond to A, T or U, G, C or mC, but preferentially pairs with C. Herein, a nucleotide containing hypoxanthine at a particular position is considered complementary to a second nucleotide containing A, T, U, C, G, or mC at the corresponding position. Complementary oligonucleotides and/or nucleic acids need not have nucleobase complementarity at each nucleoside. Rather, some mismatches are tolerated. As used herein, “fully complementary” or “100% complementary” in reference to oligonucleotides means that oligonucleotides are complementary to another oligonucleotide or nucleic acid at each nucleoside of the oligonucleotide.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that is attached to an oligonucleotide via a conjugate linker.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence. As used herein, “constrained ethyl” or “cEt” or “cEt modified sugar moiety” means a β-D ribosyl bicyclic sugar moiety wherein the second ring of the bicyclic sugar is formed via a bridge connecting the 4′-carbon and the 2′ carbon of the β-D ribosyl sugar moiety, wherein the bridge has the formula 4′ —CH(CH₃)—O—2′, and wherein the methyl group of the bridge is in the S configuration.

As used herein, “cEt” nucleoside” means a nucleoside comprising a cEt modified sugar moiety.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “gapmer” means a modified oligonucleotide comprising an internal region having a plurality of nucleosides that support RNase H cleavage positioned between external regions having one or more nucleosides, wherein the nucleosides comprising the internal region are chemically distinct from the nucleoside or nucleosides comprising the external regions. The internal region may be referred to as the “gap” and the external regions may be referred to as the “wings.” Unless otherwise indicated, “gapmer” refers to a sugar motif. Unless otherwise indicated, the sugar moieties of the nucleosides of the gap of a gapmer are unmodified β-D-2′-deoxyribosyl. Thus, the term “MOE gapmer” indicates a gapmer having a sugar motif of 2′-MOE nucleosides in both wings and a gap of β-D-2′-deoxyribonucleosides. Unless otherwise indicated, a MOE gapmer may comprise one or more modified internucleoside linkages and/or modified nucleobases and such modifications do not necessarily follow the gapmer pattern of the sugar modifications.

As used herein, “hybridization” means the pairing or annealing of complementary oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases.

As used herein, “increasing the amount or activity” refers to more transcriptional expression or activity relative to the transcriptional expression or activity in an untreated or control sample.

As used herein, “decreasing the amount or activity” refers to less transcriptional expression or activity relative to the transcriptional expression or activity in an untreated or control sample.

As used herein, “internucleoside linkage” is the covalent linkage between adjacent nucleosides in an oligonucleotide. As used herein “modified internucleoside linkage” means any internucleoside linkage other than a phosphodiester internucleoside linkage. “Phosphorothioate linkage” is a modified internucleoside linkage in which one of the non-bridging oxygen atoms of a phosphodiester internucleoside linkage is replaced with a sulfur atom.

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “non-bicyclic modified sugar moiety” means a modified sugar moiety that comprises a modification, such as a substituent, that does not form a bridge between two atoms of the sugar to form a second ring.

As used herein, “mismatch” or “non-complementary” means a nucleobase of a first oligonucleotide that is not complementary with the corresponding nucleobase of a second oligonucleotide or target nucleic acid when the first and second oligonucleotide are aligned. As used herein, a hypoxanthine (I) is not considered a mismatch to A, T, G, C, mC, or U.

As used herein, “MOE” means methoxyethyl. “2′-MOE” or “2′-MOE modified sugar moiety” means a 2′ —OCH₂CH₂OCH₃ group in place of the 2′ OH group of a ribosyl sugar moiety.

As used herein, “2′-MOE nucleoside” means a nucleoside comprising a 2′-MOE modified sugar moiety.

As used herein, “motif” means the pattern of unmodified and/or modified sugar moieties, nucleobases, and/or internucleoside linkages, in an oligonucleotide.

As used herein, “nucleobase” means an unmodified nucleobase or a modified nucleobase. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methyl cytosine” is a modified nucleobase. A universal base is a modified nucleobase that can pair with any one of the five unmodified nucleobases. Hypoxanthine (I) is a universal base.

As used herein, “nucleobase sequence” means the order of contiguous nucleobases in a nucleic acid or oligonucleotide independent of any sugar or internucleoside linkage modification.

As used herein, “nucleoside” means a compound comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are present between those that are linked).

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound. The term “oligomeric duplex” means a duplex formed by two oligomeric compounds having complementary nucleobase sequences. Each oligomeric compound of an oligomeric duplex may be referred to as a “duplexed oligomeric compound.”

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water, sterile saline, sterile buffer solution, or sterile artificial cerebrospinal fluid.

As used herein “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “phosphorus moiety” means a group of atoms comprising a phosphorus atom. In certain embodiments, a phosphorus moiety comprises a mono-, di-, or tri-phosphate, or phosphorothioate.

As used herein “prodrug” means a therapeutic agent in a form outside the body that is converted to a different form within an animal or cells thereof. Typically, conversion of a prodrug within the animal is facilitated by the action of an enzyme (e.g., endogenous or viral enzyme) or chemicals present in cells or tissues and/or by physiologic conditions.

As used herein, “RNAi compound” means an antisense compound that acts, at least in part, through RISC or Ago2 to modulate a target nucleic acid and/or protein encoded by a target nucleic acid. RNAi compounds include, but are not limited to double-stranded siRNA, single-stranded RNA (ssRNA), and microRNA, including microRNA mimics. In certain embodiments, an RNAi compound modulates the amount, activity, and/or splicing of a target nucleic acid. The term RNAi compound excludes antisense compounds that act through RNase H.

As used herein, “self-complementary” in reference to an oligonucleotide means an oligonucleotide that at least partially hybridizes to itself.

As used herein, “standard cell assay” means the assay described in Example 1 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “sugar moiety” means an unmodified sugar moiety or a modified sugar moiety. As used herein, “unmodified sugar moiety” means a 2′ —OH(H) furanosyl moiety, as found in RNA (an “unmodified RNA sugar moiety”), or a 2′ —H(H) moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. Unmodified sugar moieties are in the β-D ribosyl configuration. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or nucleic acids.

As used herein, “target nucleic acid” and “target RNA” mean a nucleic acid that an antisense compound is designed to affect. An antisense compound hybridizes to the target nucleic acid, but may comprise one or more mismatches thereto.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “therapeutically effective amount” means an amount of a pharmaceutical agent that provides a therapeutic benefit to an animal. For example, a therapeutically effective amount improves a symptom of a disease.

As used herein, “lamin A” or “lamin A protein” refers to the processed 646 amino acid lamin A protein after processing to remove the C-terminal tail.

As used herein, “LMNA” refers to the gene and the pre-mRNA gene product that produces lamin A, lamin C, and progerin. LMNA pre-mRNA nucleic acid has the sequence set forth in SEQ ID NO: 1 (GENBANK Accession No. NT_079484.1 truncated from nucleobase 2533930 to 2560103).

As used herein, “prelamin A mRNA” refers to the mRNA sequence that encodes the prelamin A protein. Wild-type prelamin A mRNA has the sequence set forth in SEQ ID NO: 2. HGPS-associated prelamin A mRNA has the sequence set forth in SEQ ID NO: 4.

As used herein, “prelamin A protein” refers to the 664 amino acid product of prelamin A mRNA, prior to removal of the C-terminal tail.

As used herein, “progerin mRNA” refers to the mRNA sequence that encodes the progerin protein. This mRNA has the sequence set forth in SEQ ID NO: 3 (GENBANK Accession No. NM_001282626.1).

As used herein, “progerin protein” refers to the 614 amino acid product of progerin mRNA. Progerin protein is farnesylated.

As used herein, “lamin C mRNA” refers to the mRNA sequence that encodes the lamin C protein. This mRNA has the sequence set forth in SEQ ID NO: 158 (GENBANK Accession No. NP_005563.1).

As used herein, “lamin C protein” refers to the protein product of the lamin C mRNA, having 572 amino acids.

The present disclosure provides the following non-limiting numbered embodiments:

Embodiment 1: An oligomeric compound comprising a modified oligonucleotide consisting of 12 to 30 linked nucleosides and having a nucleobase sequence comprising at least 12, at least 13, at least 14, at least 15, at least 16, at least 17, or at least 18 contiguous nucleobases complementary to an equal length portion of nucleobases 24759-24791 of SEQ ID NO: 1, nucleobases 2176-2198 of SEQ ID NO: 2 or SEQ ID NO:4, or nucleobases 2062-2085 of SEQ ID NO: 3.

Embodiment 2: An oligomeric compound comprising a modified oligonucleotide consisting of 12 to 30 linked nucleosides and having a nucleobase sequence comprising at least 12, at least 13, at least 14, at least 15, or at least 16 any of the nucleobase sequences of SEQ ID 14-157.

Embodiment 3: An oligomeric compound comprising a modified oligonucleotide consisting of a modified oligonucleotide having a nucleobase sequence comprising at least 17, at least 18, at least 19, or at least 20 of any of the nucleobase sequences of SEQ ID 14-38, 75-101, or 132-157

Embodiment 4: The oligomeric compound of embodiment 1, 2, or 3, wherein the modified oligonucleotide is at least 85%, at least 90%, at least 95%, or 100% complementary to SEQ ID NO: 1, SEQ ID NO: 2, SEQ ID NO: 3, or SEQ ID NO: 4 over the entire length of the modified oligonucleotide.

Embodiment 5: The oligomeric compound of any of embodiments 1-4, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, or at least 16 modified nucleoside comprising a modified sugar moiety.

Embodiment 6: The oligomeric compound of any of embodiments 1-5, wherein each nucleoside of the modified oligonucleotide comprises a modified sugar moiety.

Embodiment 7: The oligomeric compound of embodiment 5 or 6, wherein the modified sugar moiety is a 2′-methoxyethyl.

Embodiment 8: The oligomeric compound of any of embodiments 1-5, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, or at least 16 modified nucleoside comprising a bicyclic sugar moiety having a 2′-4′ bridge.

Embodiment 9: The oligomeric compound of embodiment 8, wherein the 2′-4′ bridge is selected from —O—CH₂—; and —O—CH(CH₃)—.

Embodiment 10: The oligomeric compound of embodiment 9, wherein the 2′-4′ bridge is —O—CH(CH₃)—.

Embodiment 11: The oligomeric compound of embodiment 10, wherein each nucleoside is selected from a modified nucleoside comprising a bicyclic sugar moiety having a 2′-4′ bridge or an unmodified, β-D-2′-deoxyribose nucleoside.

Embodiment 12: The oligomeric compound of embodiment 11, wherein the 2′-4′ bridge is —O—CH(CH₃)—.

Embodiment 13: The oligomeric compound of any of embodiments 1-5, wherein the modified nucleotide has a modification pattern of (A)ₘ-(A—B—B)ₙ—(A)ₒ-(B)ₚ, wherein each A is a modified nucleoside comprising a bicyclic sugar moiety having a 2′-4′ bridge, each B is a non-bicyclic nucleoside, m is 0 or 1, n is from 5-9, o is 0 or 1, and p is 0 or 1, wherein if o is 0, p is also 0.

Embodiment 14: The oligomeric compound of embodiment 13, wherein the 2′-4′ bridge is —O—CH(CH₃)—.

Embodiment 15: The oligomeric compound of embodiment 13 or 14, wherein each B is a modified nucleoside comprising a 2′-methoxyethyl modified sugar moiety.

Embodiment 16: The oligomeric compound of embodiment 13 or 14, wherein each B is an unmodified, β-D-2′-deoxyribose nucleoside.

Embodiment 17: The oligomeric compound of any of embodiments 1-16, wherein at least one internucleoside linkage of the modified oligonucleotide is a modified internucleoside linkage.

Embodiment 18: The oligomeric compound of embodiment 17, wherein the modified internucleoside linkage is a phosphorothioate internucleoside linkage.

Embodiment 19: The oligomeric compound of any of embodiments 1-16, wherein each internucleoside linkage of the modified oligonucleotide is a modified internucleoside linkage.

Embodiment 20: The oligomeric compound of embodiment 19, wherein the modified internucleoside linkage is a phosphorothioate internucleoside linkage.

Embodiment 21: The oligomeric compound of any of embodiments 1-18, wherein at least one internucleoside linkage of the modified oligonucleotide is a phosphodiester internucleoside linkage.

Embodiment 22: The oligomeric compound of any of embodiments 1-18 and 21, wherein each internucleoside linkage of the modified oligonucleotide is either a phosphodiester internucleoside linkage or a phosphorothioate internucleoside linkage.

Embodiment 23: The oligomeric compound of any of embodiments 1-5, 7-12, or 17-20, wherein the modified oligonucleotide is a gapmer.

Embodiment 24: The oligomeric compound of any of embodiments 1-22, wherein the modified oligonucleotide is not a gapmer.

Embodiment 25: The oligomeric compound of any of embodiments 1-5, 8-10, or 13, wherein the modified oligonucleotide has a sugar motif selected from among: kkddkddkddkddkddkk, kddkddkddkddkddk, kkeekeekeekeekeeke, or keekeekeekeekeek, wherein “k” represents a modified nucleoside comprising a is —O—CH(CH₃)— 2′-4′ bridge, “d” represents a β-D-2′-deoxyribose, and “e” represents nucleoside comprising a 2′-methoxyethyl modified sugar moiety.

Embodiment 26: The oligomeric compound of any of embodiments 1-25, wherein the modified oligonucleotide consists of 12-18, 12-20, 14-18, 14-20, or 16-20 linked nucleosides.

Embodiment 27: The oligomeric compound of any of embodiments 1-26, wherein the modified oligonucleotide consists of 16, 17, 18, 19, or 20 linked nucleosides.

Embodiment 28: The oligomeric compound of any of embodiments 1-27, wherein at least one nucleobase of the modified oligonucleotide comprises a modified nucleobase.

Embodiment 29: The oligomeric compound of embodiment 28, wherein the modified nucleobase is a 5-methyl cytosine.

Embodiment 30: The oligomeric compound of embodiment 28, wherein the modified nucleobase is hypoxanthine

Embodiment 31: The oligomeric compound of any of embodiments 1-5, wherein each nucleobase is selected from among adenine, guanine, cytosine, thymine, or 5-methyl cytosine.

Embodiment 32: The oligomeric compound of any of embodiments 1-5, wherein each nucleobase is selected from among adenine, guanine, cytosine, thymine, 5-methyl cytosine, or hypoxanthine.

Embodiment 33: The oligomeric compound of embodiment 32, wherein each nucleoside comprising adenine, guanine, cytosine, thymine, or 5-methyl cytosine comprises a 2′-modified sugar moiety, and wherein each nucleoside comprising hypoxanthine comprises a β-D-2′-deoxyribose.

Embodiment 34: The oligomeric compound of embodiment 33, wherein the modified sugar moiety is a 2′-methoxyethyl.

Embodiment 35: The oligomeric compound of any of embodiments 1-34, consisting of the modified oligonucleotide.

Embodiment 36: The oligomeric compound of any of embodiments 1-34, comprising a conjugate group comprising a conjugate moiety and a conjugate linker.

Embodiment 37: The oligomeric compound of embodiment 36, wherein the conjugate moiety comprises a lipophilic group.

Embodiment 38: The oligomeric compound of embodiment 37, wherein the conjugate moiety is selected from among: cholesterol, C10-C26 saturated fatty acid, C10-C26 unsaturated fatty acid, C10-C26 alkyl, triglyceride, tocopherol, or cholic acid.

Embodiment 39: The oligomeric compound of embodiment 38, wherein the conjugate moiety is a saturated fatty acid or an unsaturated fatty acid.

Embodiment 40: The oligomeric compound of embodiment 38, wherein the conjugate moiety is C16 alkyl.

Embodiment 41: The oligomeric compound of any of embodiments 36-40, wherein the conjugate linker consists of a single bond.

Embodiment 42: The oligomeric compound of any of embodiments 36-40, wherein the conjugate linker is cleavable.

Embodiment 43: The oligomeric compound of any of embodiments 36-40, wherein the conjugate linker comprises 1-3 linker nucleosides.

Embodiment 44: The oligomeric compound of embodiment 43, wherein the oligomeric compound comprises no more than 24 total linked nucleosides, including the modified oligonucleotide and linker nucleosides.

Embodiment 45: The oligomeric compound of any of embodiments 36-40, wherein the conjugate linker comprises a hexylamino group.

Embodiment 46: The oligomeric compound of any of embodiments 36-40, wherein the conjugate linker comprises a polyethylene glycol group.

Embodiment 47: The oligomeric compound of any of embodiments 36-40, wherein the conjugate linker comprises a triethylene group.

Embodiment 48: The oligomeric compound of any of embodiments 36-40, wherein the conjugate linker comprises a phosphate group.

Embodiment 49: The oligomeric compound of embodiment 36, wherein the conjugate group has formula I:

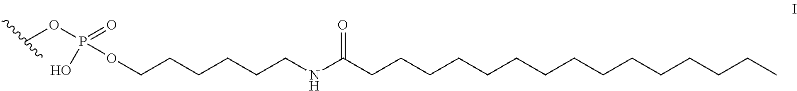 [SMILES: *OP(=O)(O)OCCCCCCNC(=O)CCCCCCCCCCCCCCC]

Embodiment 50: The oligomeric compound of any of embodiments 1-49, wherein the oligomeric compound is single-stranded.

Embodiment 51: An oligomeric duplex comprising any oligomeric compound of any of embodiments 1-49.

Embodiment 52: An antisense compound comprising or consisting of an oligomeric compound of any of embodiments 1-50 or an oligomeric duplex of embodiment 51.

Embodiment 53: A pharmaceutical composition comprising an oligomeric compound of any of embodiments 1-50, an oligomeric duplex of embodiment 51, or an antisense compound of embodiment 52, and at least one of a pharmaceutically acceptable carrier or diluent.

Embodiment 54: The pharmaceutical composition of embodiment 53, wherein the modified oligonucleotide is a sodium salt.

Embodiment 55: A method comprising administering to an animal the pharmaceutical composition of embodiment 53 or 54.

Embodiment 56: The method of embodiment 55, wherein the animal is a human.

Embodiment 57: A method of treating a disease associated with LMNA comprising administering to an individual having or at risk of developing a disease associated with LMNA a therapeutically effective amount of a pharmaceutical composition of embodiments 53 or 54.

Embodiment 58: The method of embodiment 56, wherein the disease is Hutchinson-Gilford Progeria Syndrome

Embodiment 59: The method of embodiment 57, wherein at least one symptom of Hutchinson-Gilford Progeria Syndrome is ameliorated.

Embodiment 60: The method of embodiment 59, wherein the symptom is weight loss.

Embodiment 61: The method of embodiment 59, wherein the symptoms is premature death.

Embodiment 62: A method comprising the co-administration of two or more oligomeric compounds of any of embodiments 1-50 to an individual.

Embodiment 63: A method comprising the concomitant administration of two or more oligomeric compounds of any of embodiments 1-50 to an individual.

### Certain Oligonucleotides

In certain embodiments, provided herein are oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage.

### Certain Modified Nucleosides

Modified nucleosides comprise a modified sugar moiety or a modified nucleobase or both a modified sugar moiety and a modified nucleobase.

### Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified sugar moieties comprising a furanosyl ring with one or more substituent groups none of which bridges two atoms of the furanosyl ring to form a bicyclic structure. Such non bridging substituents may be at any position of the furanosyl, including but not limited to substituents at the 2′, 4′, and/or 5′ positions. In certain embodiments one or more non-bridging substituent of non-bicyclic modified sugar moieties is branched. Examples of 2′-substituent groups suitable for non-bicyclic modified sugar moieties include but are not limited to: 2′ —F, 2′ —OCH₃(“OMe” or “O-methyl”), and 2′ —O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkynyl—O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl. Examples of 4′-substituent groups suitable for non-bicyclic modified sugar moieties include but are not limited to alkoxy (e.g., methoxy), alkyl, and those described in Manoharan et al., WO 2015/106,128. Examples of 5′-substituent groups suitable for non-bicyclic modified sugar moieties include but are not limited to: 5′-methyl (R or S), 5′-vinyl, and 5′-methoxy. In certain embodiments, non-bicyclic modified sugar moieties comprise more than one non-bridging sugar substituent, for example, 2′ —F— 5′-methyl sugar moieties and the modified sugar moieties and modified nucleosides described in Migawa et al., WO 2008/101,157 and Rajeev et al., US2013/0203836.).

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted nucleoside non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

Certain modified sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′ —CH₂-2′, 4′ —(CH₂)₂-2′, 4′ —(CH₂)₃-2′, 4′ —CH₂—O-2′ (“LNA”), 4′ —CH₂—S— 2′, 4′-(CH₂)₂—O— 2′ (“ENA”), 4′ —CH(CH₃)—O-2′ (referred to as “constrained ethyl” or “cEt”), 4′ —CH₂—O—CH₂-2′, 4′ —CH₂—N(R)— 2′, 4′ —CH(CH₂OCH₃)—O— 2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′ —C(CH₃)(CH₃)—O— 2′ and analogs thereof (see, e.g., Seth et al., U.S. Pat. No. 8,278,283), 4′ —CH₂—N(OCH₃)— 2′ and analogs thereof (see, e.g., Prakash et al., U.S. Pat. No. 8,278,425), 4′ —CH₂—O—N(CH₃)— 2′ (see, e.g., Allerson et al., U.S. Pat. No. 7,696,345 and Allerson et al., U.S. Pat. No. 8,124,745), 4′ —CH₂—C(H)(CH₃)— 2′ (see, e.g., Zhou, et al., J. Org. Chem., 2009, 74, 118-134), 4′ —CH₂—C(═CH₂)— 2′ and analogs thereof (see e.g., Seth et al., U.S. Pat. No. 8,278,426), 4′ —C(RₐR_{b})—N(R)—O— 2′, 4′ —C(RₐR_{b})—O—N(R)— 2′, 4′ —CH₂—O—N(R)— 2′, and 4′ —CH₂—N(R)—O— 2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]ₙ—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2017, 129, 8362-8379; Wengel et a., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; and Ramasamy et al., U.S. Pat. No. 6,525,191; Torsten et al., WO 2004/106356; Wengel et al., WO 1999/014226; Seth et al., WO 2007/134,181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S.

8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

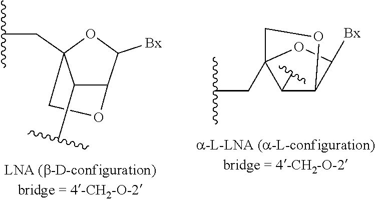

α-L-methyleneoxy (4′ —CH₂—O-2′) or α-L-LNA bicyclic nucleosides have been incorporated into oligonucleotides that showed antisense activity (Frieden et al., Nucleic Acids Research, 2003, 21, 6365-6372). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

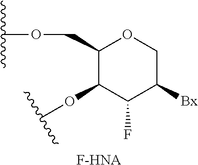

“F—HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F—HNA can also be referred to as a F-THP or 3′-fluoro tetrahydropyran), and nucleosides comprising additional modified THP compounds having the formula:

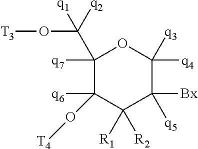

wherein, independently, for each of said modified THP nucleoside:

Bx is a nucleobase moiety;

T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group; q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and

each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

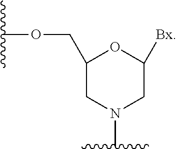

In certain embodiments, morpholinos may be modified, for example by adding or altering various substituent groups from the above morpholino structure. Such sugar surrogates are referred to herein as “modified morpholinos.”

In certain embodiments, sugar surrogates comprise acyclic moieties. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876.

Many other bicyclic and tricyclic sugar and sugar surrogate ring systems are known in the art that can be used in modified nucleosides).

### Certain Modified Nucleobases

In certain embodiments, modified oligonucleotides comprise one or more nucleoside comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and O-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azauracil, 6-azacytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manohara et al., US2003/0158403; Manoharan et al., US2003/0175906; Dinh et al., U.S. Pat. No. 4,845,205; Spielvogel et al., U.S. Pat. No. 5,130,302; Rogers et al., U.S. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### Certain Modified Internucleoside Linkages

In certain embodiments, nucleosides of modified oligonucleotides may be linked together using any internucleoside linkage. The two main classes of internucleoside linking groups are defined by the presence or absence of a phosphorus atom. Representative phosphorus-containing internucleoside linkages include but are not limited to phosphates, which contain a phosphodiester bond (“P═O”) (also referred to as unmodified or naturally occurring linkages), phosphotriesters, methylphosphonates, phosphoramidates, and phosphorothioates (“P═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphate linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such chirally enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

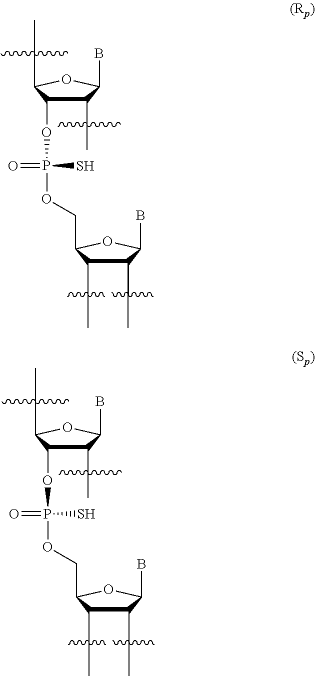

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′ —CH₂—N(CH₃)—O-5′), amide-3 (3′ —CH₂—C(═O)—N(H)-5′), amide-4 (3′ —CH₂—N(H)—C(═O)-5′), formacetyl (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

### Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### Certain Sugar Motifs

In certain embodiments, oligonucleotides comprise one or more type of modified sugar and/or unmodified sugar moiety arranged along the oligonucleotide or region thereof in a defined pattern or sugar motif. In certain instances, such sugar motifs include but are not limited to any of the sugar modifications discussed herein.

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-5 nucleosides. In certain embodiments, each nucleoside of each wing of a gapmer is a modified nucleoside. In certain embodiments, at least one nucleoside of each wing of a gapmer is a modified nucleoside. In certain embodiments, at least two nucleosides of each wing of a gapmer are modified nucleosides. In certain embodiments, at least three nucleosides of each wing of a gapmer are modified nucleosides. In certain embodiments, at least four nucleosides of each wing of a gapmer are modified nucleosides.

In certain embodiments, the gap of a gapmer comprises 7-12 nucleosides. In certain embodiments, each nucleoside of the gap of a gapmer is an unmodified β-D-2′-deoxy nucleoside.

In certain embodiments, the gapmer is a deoxy gapmer. In embodiments, the nucleosides on the gap side of each wing/gap junction are unmodified β-D-2′-deoxy nucleosides and the nucleosides on the wing sides of each wing/gap junction are modified nucleosides. In certain embodiments, each nucleoside of the gap is an unmodified β-D-2′-deoxy nucleoside. In certain embodiments, each nucleoside of each wing of a gapmer is a modified nucleoside.

Herein, the lengths (number of nucleosides) of the three regions of a gapmer may be provided using the notation [# of nucleosides in the 5′-wing]−[# of nucleosides in the gap]−[# of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprise unmodified β-D-2′-deoxyribonucleoside sugars. Thus, a 5-10-5 MOE gapmer consists of 5 linked MOE modified nucleosides in the 5′-wing, 10 linked β-D-2′-deoxyribonucleosides in the gap, and 5 linked MOE nucleosides in the 3′-wing.

In certain embodiments, modified oligonucleotides are 5-10-5 MOE gapmers. In certain embodiments, modified oligonucleotides are 3-10-3 BNA gapmers. In certain embodiments, modified oligonucleotides are 3-10-3 cEt gapmers. In certain embodiments, modified oligonucleotides are 3-10-3 LNA gapmers.

In certain embodiments, modified oligonucleotides comprise or consist of a region having a fully modified sugar motif. In such embodiments, each nucleoside of the fully modified region of the modified oligonucleotide comprises a modified sugar moiety. In certain embodiments, modified oligonucleotides comprise or consist of a region having a fully modified sugar motif, wherein each nucleoside within the fully modified region comprises the same modified sugar moiety (uniformly modified sugar motif). In certain embodiments, the uniformly modified sugar motif is 7 to 20 nucleosides in length. In certain embodiments, each nucleoside of the uniformly modified sugar motif is a 2′-substituted nucleoside, a sugar surrogate, or a bicyclic nucleoside. In certain embodiments, each nucleoside of the uniformly modified sugar motif comprises either a 2′ —OCH₂CH₂OCH₃ group or a 2′ —OCH₃ group. In certain embodiments, modified oligonucleotides having at least one fully modified sugar motif may also have at least 1, at least 2, at least 3, or at least 4 2′-deoxyribonucleosides.

In certain embodiments, each nucleoside of the entire modified oligonucleotide comprises a modified sugar moiety (fully modified oligonucleotide). In certain embodiments, a fully modified oligonucleotide comprises different 2′-modifications. In certain embodiments, each nucleoside of a fully modified oligonucleotide is a 2′-substituted nucleoside, a sugar surrogate, or a bicyclic nucleoside. In certain embodiments, each nucleoside of a fully modified oligonucleotide comprises either a 2′ —OCH₂CH₂OCH₃ group and at least one 2′ —OCH₃ group.

In certain embodiments, each nucleoside of a fully modified oligonucleotide comprises the same 2′-modification (uniformly modified oligonucleotide). In certain embodiments, each nucleoside of a uniformly modified oligonucleotide is a 2′-substituted nucleoside, a sugar surrogate, or a bicyclic nucleoside. In certain embodiments, each nucleoside of a uniformly modified oligonucleotide comprises either a 2′ —OCH₂CH₂OCH₃ group or a 2′ —OCH₃ group

In certain embodiments, modified oligonucleotides comprise at least 12, at last 13, at least 14, at least 15, at least 16, at least 17, at least 18, at least 19, or at least 20 nucleosides comprising a modified sugar moiety. In certain embodiments, each nucleoside of a modified oligonucleotide is a 2′-substituted nucleoside, a sugar surrogate, a bicyclic nucleoside, or a β-D-2′-deoxyribonucleoside. In certain embodiments, each nucleoside of a modified oligonucleotide comprises a 2′ —OCH₂CH₂OCH₃ group, a 2′-H(H) deoxyribosyl sugar moiety, or a cEt modified sugar.

In certain embodiments, modified oligonucleotides comprise a modification pattern of (A)ₘ-(A—B—B)ₙ—(A)ₒ-(B)ₚ, wherein each A is a modified nucleoside comprising a bicyclic sugar moiety having a 2′-4′ bridge, each B is a non-bicyclic nucleoside, m is 0 or 1, n is from 5-9, o is 0, 1, or 2 and p is 0 or 1, wherein if o is 0, p is also 0. Examples of sugar motifs represented by this formula are exemplified in the table below.

TABLE 1
Sugar Motifs

| A | B | m | n | o | p | length | sugar motif |
|---|---|---|---|---|---|---|---|
| k | d | 1 | 5 | 2 | 0 | 18 | kkddkddkddkddkddkk |
| k | d | 0 | 5 | 1 | 0 | 16 | kddkddkddkddkddk |
| k | e | 1 | 5 | 1 | 1 | 18 | kkeekeekeekeekeeke |
| k | e | 0 | 5 | 1 | 0 | 16 | keekeekeekeekeek |

In the table above, “k” represents modified nucleoside with a bicyclic sugar moiety having a —O—CH(CH₃)—2′-4′ bridge (cEt), “e” represents a 2′-methoxyethyl modified nucleoside, and “d” represents a β-D-2′-deoxyribose nucleoside.

In certain embodiments, modified oligonucleotides comprise a modification pattern of (C)ₘ—(C-D)ₙ-(C)ₒ, wherein m is 0 or 1, n is 7 to 12, and o is 0-2. When m is 0, n is 9, and o is 2, and C is a modified nucleoside comprising a 2′-methoxyethyl modified sugar moiety and D is a β-D-2′-deoxyribonucleoside, this modification pattern can also be represented by the sugar motif notation edededededededededee, wherein “e” represents a 2′-methoxyethyl modified nucleoside, and “d” represents a 2′-deoxyribose nucleoside.

### Certain Nucleobase Motifs

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methyl cytosines. In certain embodiments, all of the cytosine nucleobases are 5-methyl cytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of said nucleoside is a β-D-2′-deoxyribosyl moiety. In certain embodiments, the modified nucleobase is selected from: a 2-thiopyrimidine and a 5-propynepyrimidine. In certain embodiments, the modified nucleobase is a hypoxanthine

### Certain Internucleoside Linkage Motifs

In certain embodiments, oligonucleotides comprise modified and/or unmodified internucleoside linkages arranged along the oligonucleotide or region thereof in a defined pattern or motif. In certain embodiments, each internucleoside linking group is a phosphodiester internucleoside linkage (P═O). In certain embodiments, each internucleoside linking group of a modified oligonucleotide is a phosphorothioate internucleoside linkage (P═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate a (Sp) phosphorothioate, and a (Rp) phosphorothioate. In certain embodiments, the sugar motif of a modified oligonucleotide is a gapmer and the internucleoside linkages within the gap are all modified. In certain such embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester linkage is not a terminal internucleoside linkage, and the remaining internucleoside linkages are phosphorothioate internucleoside linkages. In certain such embodiments, all of the phosphorothioate linkages are stereorandom. In certain embodiments, all of the phosphorothioate linkages in the wings are (Sp) phosphorothioates, and the gap comprises at least one Sp, Sp, Rp motif. In certain embodiments, populations of modified oligonucleotides are enriched for modified oligonucleotides comprising such internucleoside linkage motifs.

### Certain Lengths

It is possible to increase or decrease the length of an oligonucleotide without eliminating activity. For example, in Woolf et al. (Proc. Natl. Acad. Sci. USA 89: 7305-7309, 1992), a series of oligonucleotides 13-25 nucleobases in length were tested for their ability to induce cleavage of a target RNA in an oocyte injection model. Oligonucleotides 25 nucleobases in length with 8 or 11 mismatch bases near the ends of the oligonucleotides were able to direct specific cleavage of the target RNA, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

### Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

In certain embodiments, modified oligonucleotides are uniformly modified with a 2′-methoxyethyl at all positions other than one or two positions comprising a hypoxanthine nucleobase. In certain such embodiments, the nucleoside comprising hypoxanthine comprises a β-D-2′-deoxyribosyl sugar moiety.

### Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a region of an oligonucleotide has a nucleobase sequence that is complementary to a second oligonucleotide or an identified reference nucleic acid, such as a target nucleic acid. In certain embodiments, the nucleobase sequence of a region or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2′-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, modified or unmodified nucleosides, and two or more nucleosides that are independently modified or unmodified.

### Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance. In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di—O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecyLamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), a GalNAc cluster (e.g., WO2014/179620). Other targeting groups are described in WO/2017/053995, hereby incorporated by reference.

In certain embodiments, the conjugate group has formula I:

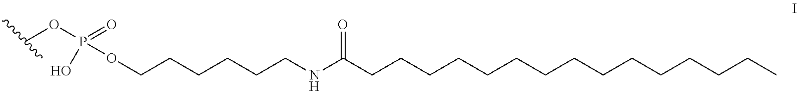 [SMILES: *OP(=O)(O)OCCCCCCNC(=O)CCCCCCCCCCCCCCC]

### Conjugate Moieties

Conjugate moieties include, without limitation, intercalators, reporter molecules, polyamines, polyamides, peptides, carbohydrates, vitamin moieties, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

In certain embodiments, a conjugate moiety is selected from among: cholesterol, C10-C26 saturated fatty acid, C10-C26 unsaturated fatty acid, C10-C26 alkyl, triglyceride, tocopherol, or cholic acid. In certain embodiments, a conjugate moiety is C16 alkyl.

### Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methyl cytosine, 4-N-benzoyl-S-methyl cytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is a 2′-deoxy nucleoside that is attached to either the 3′ or 5′-terminal nucleoside of an oligonucleotide by a phosphate internucleoside linkage and covalently attached to the remainder of the conjugate linker or conjugate moiety by a phosphate or phosphorothioate linkage. In certain such embodiments, the cleavable moiety is 2′-deoxyadenosine.

### Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphonates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### Oligomeric Duplexes

In certain embodiments, oligomeric compounds described herein comprise an oligonucleotide, having a nucleobase sequence complementary to that of a target nucleic acid. In certain embodiments, an oligomeric compound is paired with a second oligomeric compound to form an oligomeric duplex. Such oligomeric duplexes comprise a first oligomeric compound having a region complementary to a target nucleic acid and a second oligomeric compound having a region complementary to the first oligomeric compound. In certain embodiments, the first oligomeric compound of an oligomeric duplex comprises or consists of (1) a modified or unmodified oligonucleotide and optionally a conjugate group and (2) a second modified or unmodified oligonucleotide and optionally a conjugate group. Either or both oligomeric compounds of an oligomeric duplex may comprise a conjugate group. The oligonucleotides of each oligomeric compound of an oligomeric duplex may include non-complementary overhanging nucleosides.

### Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they increase the amount or activity of a target nucleic acid by 25% or more in the standard cell assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi compounds. RNAi compounds may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid.

In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid. In certain embodiments, hybridization of an antisense compound to a target nucleic acid results in an increase in the amount or activity of a target nucleic acid.

Use of oligomeric compounds is an effective means for modulating the expression of one or more specific gene products and is uniquely useful in a number of therapeutic, diagnostic, and research applications. Provided herein are oligomeric compounds useful for modulating gene expression via antisense mechanisms of action, including antisense mechanisms based on target occupancy. In certain embodiments, the oligomeric compounds provided herein modulate splicing of a target gene.

In certain embodiments, an antisense compound is complementary to a region of an LMNA pre-mRNA. In certain embodiments, a modified oligonucleotide modulates splicing of a pre-mRNA. In certain embodiments, a modified oligonucleotide modulates splicing of an LMNA pre-mRNA. In certain such embodiments, the LMNA pre-mRNA is transcribed from a mutant variant of LMNA. In certain embodiments, the mutant variant comprises an aberrant splice site. In certain embodiments, the aberrant splice site of the mutant variant comprises a mutation that induces a cryptic 5′ splice site. In certain embodiments, a modified oligonucleotide reduces progerin mRNA. In certain embodiments, a modified oligonucleotide increases the production of lamin C mRNA or protein while reducing progerin mRNA or protein.

### Certain Target Nucleic Acids

In certain embodiments, oligomeric compounds comprise or consist of an oligonucleotide comprising a region that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature RNA and a pre-mRNA, including intronic, exonic and untranslated regions. In certain embodiments, the target RNA is a mature RNA. In certain embodiments, the target nucleic acid is a pre-mRNA. In certain such embodiments, the target region is entirely within an intron. In certain embodiments, the target region spans an intron/exon junction. In certain embodiments, the target region is at least 50% within an intron. In certain embodiments, the target nucleic acid has a disease-associated mutation. In certain embodiments, the target nucleic acid is the RNA transcriptional product of a retrogene. In certain embodiments, the target nucleic acid is a non-coding RNA. In certain such embodiments, the target non-coding RNA is selected from: a long non-coding RNA, a short non-coding RNA, an intronic RNA molecule.

### Complementarity/Mismatches to the Target Nucleic Acid

It is possible to introduce mismatch bases without eliminating activity. For example, Gautschi et al (J. Natl. Cancer Inst. 93: 463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16: 3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides that are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, 12 to 14, 12 to 16, 14 to 16, 16 to 18, or 18 to 20 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, or 8 from the 5′-end of the gap region. In certain embodiments, the mismatch is at position 9, 8, 7, 6, 5, 4, 3, 2, 1 from the 3′-end of the gap region. In certain embodiments, the mismatch is at position 1, 2, 3, or 4 from the 5′-end of the wing region. In certain embodiments, the mismatch is at position 4, 3, 2, or 1 from the 3′-end of the wing region.

### LMNA

In certain embodiments, oligomeric compounds comprise or consist of an oligonucleotide comprising a region that is complementary to a target nucleic acid, wherein the target nucleic acid is LMNA. In certain embodiments, LMNA nucleic acid has the sequence set forth in SEQ ID NO: 1 (GENBANK Accession No. NT_079484.1 truncated from nucleobase 2533930 to 2560103). In certain embodiments, the target nucleic acid is prelamin mRNA as set forth in SEQ ID NO: 2 (GENBANK Accession No. NM_170707.1) or SEQ ID NO: 4. In certain embodiments, the target nucleic acid is progerin mRNA as set forth in SEQ ID NO: 3 (GENBANK Accession No. NM_001282626.1). In certain embodiments, prelamin A mRNA associated with HGPS has the sequence set forth in SEQ ID NO: 4. SEQ ID NO: 4 is identical to SEQ ID NO:2 aside from a C to T mutation at position 2036.

TABLE 2
LMNA Isoforms

| | | | Protein | Amino acid | Protein product | Amino acids |
|---|---|---|---|---|---|---|
| | mRNA | SEQ | product | in | after post- | after post- |
| mRNA | Accession | ID | before | translated | translational | translational |
| name | # | NO: | processing | product | processing | processing |
| wild-type | NM_170707.1 | 2 | prelamin A | 664 | lamin A | 646 |
| prelamin A | | | | | | |
| HGPS- | N/A | 4 | prelamin A* | 664 | lamin A | 646 |
| associated | | | | | | |
| lamin A | | | | | | |
| progerin | NM_001282626.1 | 3 | progerin | 614 | N/A | N/A |
| lamin C | NP_005563.1 | 158 | lamin C | 572 | N/A | N/A |

*The mutation most commonly associated with HGPS does not change the protein sequence of the translated prelamin A protein. Instead, the mutation changes the splicing ratio of progerin and prelamin A. It is a silent mutation on the protein level such that the prelamin A protein that is produced is identical.

In certain embodiments, contacting a cell with an oligomeric compound complementary to SEQ ID NO: 1, SEQ ID NO: 2, SEQ ID NO: 3, or SEQ ID NO: 4 increases the amount of prelamin A mRNA, and in certain embodiments increases the amount of Lamin A protein. In certain embodiments, contacting a cell with an oligomeric compound complementary to SEQ ID NO: 1, or SEQ ID NO: 2, SEQ ID NO: 3, or SEQ ID NO: 4 decreases the amount of progerin mRNA, and in certain embodiments decreases the amount of progerin protein. In certain embodiments, contacting a cell with an oligomeric compound comprising a modified oligonucleotide complementary to SEQ ID NO: 1, or SEQ ID NO: 2, SEQ ID NO: 3, or SEQ ID NO: 4 selectively decreases the amount of progerin mRNA and/or protein relative to prelamin A mRNA and/or lamin A protein. In certain embodiments, contacting a cell with an oligomeric compound comprising a modified oligonucleotide complementary to SEQ ID NO: 1, or SEQ ID NO: 2, SEQ ID NO: 3, or SEQ ID NO: 4 decreases progerin mRNA and/or protein and increases lamin C mRNA and/or lamin C protein.

In certain embodiments, contacting a cell in an animal with an oligomeric compound complementary SEQ ID NO: 1, or SEQ ID NO: 2, SEQ ID NO: 3, or SEQ ID NO: 4 ameliorates one or more symptoms of HGPS. Such symptoms include a lack of subcutaneous fat, sclerotic skin, joint contractures, bone abnormalities, weight loss, hair loss, hypertension, metabolic syndrome, central nervous system sequelae, conductive hearing loss, oral deficits, craniofacial abnormalities, progressive cardiovascular disease resembling atherosclerosis, congestive heart failure, and premature death. In certain embodiments, contacting a cell in an animal with an oligonucleotide complementary to SEQ ID NO: 1, or SEQ ID NO: 2, SEQ ID NO: 3, or SEQ ID NO: 4 results in reduced weight loss and prolonged survival.

### Certain Target Nucleic Acids in Certain Tissues

In certain embodiments, oligomeric compounds comprise or consist of an oligonucleotide comprising a region that is complementary to a target nucleic acid, wherein the target nucleic acid is expressed in a pharmacologically relevant tissue. In certain embodiments, the pharmacologically relevant tissues are the cells and tissues that comprise the liver, bladder, kidneys, lungs, stomach, intestines, vasculature, skeletal muscle, and cardiac muscle.

### Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid. In certain embodiments, the artificial cerebrospinal fluid is pharmaceutical grade.

In certain embodiments, a pharmaceutical composition comprises a modified oligonucleotide and artificial cerebrospinal fluid. In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to an animal, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue.

In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal, intracerebroventricular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Nonlimiting disclosure and incorporation by reference
Each of the literature and patent publications listed herein is incorporated by reference in its entirety. While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.
Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar (2′-OH in place of one 2′-H of DNA) or as an RNA having a modified base (thymine (methylated uracil) in place of an uracil of RNA). Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “ATmCGAUCG,” wherein mC indicates a cytosine base comprising a methyl group at the 5-position.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as a or 13 such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry included all such possible isomers, including their stereorandom and optically pure forms, unless specified otherwise. Likewise, all tautomeric forms of the compounds herein are also included unless otherwise indicated. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments. For example, disclosure of an oligonucleotide having a particular motif provides reasonable support for additional oligonucleotides having the same or similar motif. And, for example, where a particular high-affinity modification appears at a particular position, other high-affinity modifications at the same position are considered suitable, unless otherwise indicated.

### Example 1 Effect of Modified Oligonucleotides Complementary to the Human Progerin 5′-Splice Site, in Vitro

Modified oligonucleotides complementary to the human progerin 5′-splice site were designed and tested for their effect on progerin mRNA, prelamin A mRNA, and lamin C mRNA in vitro in HGPS patient-derived fibroblasts. Modified oligonucleotides in the table below are complementary to wild-type human LMNA pre-mRNA (SEQ ID NO: 1) or the mutant prelamin A mRNA having the HGPS-associated G608G mutation (SEQ ID NO: 4). The oligonucleotides comprise 2′-4′-constrained ethyl (cEt) nucleosides, 2′-methoxyethyl nucleosides, and β-D-2′-deoxyribonucleosides and each internucleoside linkage is a phosphorothioate linkage. Each cytosine residue is a 5-methyl cytosine. The sugar motif of each modified oligonucleotide is provided in the sugar motif column of Table 5 below.

Nucleosides that are underlined represent a single nucleoside mismatch to the wild-type human genomic sequence of LMNA L(SEQ ID NO:1) at that position. In the table below, each underlined nucleoside is an adenine, and it is aligned with a cytosine in SEQ ID NO:1. Compounds are 100% complementary to SEQ ID NO: 4.

TABLE 3
Modified oligonucleotides complementary to
the human progerin 5′-splice site

| | | SEQ ID | SEQ ID | SEQ ID | SEQ ID | SEQ |
|---|---|---|---|---|---|---|
| Compound | Sequence | NO: 1 | NO: 1 | NO: 4 | NO: 4 | ID |
| ID | (5′ to 3′) | start site | stop site | start site | stop site | NO: |
| 586000 | AGCACGGTGCGCGAGCGC | 24226 | 24243 | 1955 | 1972 | 14 |
| 586001 | CGCACAGCACGGTGCGCG | 24231 | 24248 | 1960 | 1977 | 15 |
| 586002 | GGTCCCGCACAGCACGGT | 24236 | 24253 | 1965 | 1982 | 16 |
| 586003 | CCGCAGGTCCCGCACAGC | 24241 | 24258 | 1970 | 1987 | 17 |
| 586004 | GCTGCCCGCAGGTCCCGC | 24246 | 24263 | 1975 | 1992 | 18 |
| 586005 | GGCAGGCTGCCCGCAGGT | 24251 | 24268 | 1980 | 1997 | 19 |
| 586006 | TTGTCGGCAGGCTGCCCG | 24256 | 24273 | 1985 | 2002 | 20 |
| 586007 | ATGCCTTGTCGGCAGGCT | 24261 | 24278 | 1990 | 2007 | 21 |
| 586008 | GGCAGATGCCTTGTCGGC | 24266 | 24283 | 1995 | 2012 | 22 |
| 586009 | CCGCTGGCAGATGCCTTG | 24271 | 24288 | 2000 | 2017 | 23 |
| 586010 | CTGAGCCGCTGGCAGATG | 24276 | 24293 | 2005 | 2022 | 24 |
| 586011 | GGCTCCTGAGCCGCTGGC | 24281 | 24298 | 2010 | 2027 | 25 |
| 586012 | TGGGCTCCTGAGCCGCTG | 24283 | 24300 | 2012 | 2029 | 26 |
| 586013 | CCTGGGCTCCTGAGCCGC | 24285 | 24302 | 2014 | 2031 | 27 |
| 586014 | CACCTGGGCTCCTGAGCC | 24287 | 24304 | 2016 | 2033 | 28 |
| 586015 | CCCACCTGGGCTCCTGAG | 24289 | 24306 | 2018 | 2035 | 29 |
| 586016 | CACCCACCTGGGCTCCTG | 24291 | 24308 | 2020 | 2037 | 30 |
| 586017 | TCCACCCACCTGGGCTCC | 24293 | 24310 | 2022 | 2039 | 31 |
| 586018 | GGTCCACCCACCTGGGCT | 24295 | 24312 | 2024 | 2041 | 32 |
| 586019 | TGGGTCCACCCACCTGGG | 24297 | 24314 | 2026 | 2043 | 33 |
| 586020 | GATGGGTCCACCCACCTG | 24299 | 24316 | 2028 | 2045 | 34 |
| 586021 | GAGATGGGTCCACCCACC | 24301 | 24318 | 2030 | 2047 | 35 |
| 586022 | AGGAGATGGGTCCACCCA | 24303 | 24320 | 2032 | 2049 | 36 |
| 586023 | AGAGGAGATGGGTCCACC | 24305 | 24322 | 2034 | 2051 | 37 |
| 586024 | CCAGAGGAGATGGGTCCA | 24307 | 24324 | 2036 | 2053 | 38 |
| 586025 | CCCACCTGGGCTCCTG | 24291 | 24306 | 2020 | 2035 | 39 |
| 586026 | CACCCACCTGGGCTCC | 24293 | 24308 | 2022 | 2037 | 40 |
| 586027 | TCCACCCACCTGGGCT | 24295 | 24310 | 2024 | 2039 | 41 |
| 586028 | GGTCCACCCACCTGGG | 24297 | 24312 | 2026 | 2041 | 42 |
| 586029 | TGGGTCCACCCACCTG | 24299 | 24314 | 2028 | 2043 | 43 |
| 586030 | AGCACGGTGCGCGAGCGC | 24226 | 24243 | 1955 | 1972 | 14 |
| 586031 | CGCACAGCACGGTGCGCG | 24231 | 24248 | 1960 | 1977 | 15 |
| 586032 | GGTCCCGCACAGCACGGT | 24236 | 24253 | 1965 | 1982 | 16 |
| 586033 | CCGCAGGTCCCGCACAGC | 24241 | 24258 | 1970 | 1987 | 17 |
| 586034 | GCTGCCCGCAGGTCCCGC | 24246 | 24263 | 1975 | 1992 | 18 |
| 586035 | GGCAGGCTGCCCGCAGGT | 24251 | 24268 | 1980 | 1997 | 19 |
| 586036 | TTGTCGGCAGGCTGCCCG | 24256 | 24273 | 1985 | 2002 | 20 |
| 586037 | ATGCCTTGTCGGCAGGCT | 24261 | 24278 | 1990 | 2007 | 21 |
| 586038 | GGCAGATGCCTTGTCGGC | 24266 | 24283 | 1995 | 2012 | 22 |
| 586039 | CCGCTGGCAGATGCCTTG | 24271 | 24288 | 2000 | 2017 | 23 |
| 586040 | CTGAGCCGCTGGCAGATG | 24276 | 24293 | 2005 | 2022 | 24 |
| 586041 | GGCTCCTGAGCCGCTGGC | 24281 | 24298 | 2010 | 2027 | 25 |
| 586042 | TGGGCTCCTGAGCCGCTG | 24283 | 24300 | 2012 | 2029 | 26 |
| 586043 | CCTGGGCTCCTGAGCCGC | 24285 | 24302 | 2014 | 2031 | 27 |
| 586044 | CACCTGGGCTCCTGAGCC | 24287 | 24304 | 2016 | 2033 | 28 |
| 586045 | CCCACCTGGGCTCCTGAG | 24289 | 24306 | 2018 | 2035 | 29 |
| 586046 | CACCCACCTGGGCTCCTG | 24291 | 24308 | 2020 | 2037 | 30 |
| 586047 | TCCACCCACCTGGGCTCC | 24293 | 24310 | 2022 | 2039 | 31 |
| 586048 | GGTCCACCCACCTGGGCT | 24295 | 24312 | 2024 | 2041 | 32 |
| 586049 | TGGGTCCACCCACCTGGG | 24297 | 24314 | 2026 | 2043 | 33 |
| 586050 | GATGGGTCCACCCACCTG | 24299 | 24316 | 2028 | 2045 | 34 |
| 586051 | GAGATGGGTCCACCCACC | 24301 | 24318 | 2030 | 2047 | 35 |
| 586052 | AGGAGATGGGTCCACCCA | 24303 | 24320 | 2032 | 2049 | 36 |
| 586053 | AGAGGAGATGGGTCCACC | 24305 | 24322 | 2034 | 2051 | 37 |
| 586054 | CCAGAGGAGATGGGTCCA | 24307 | 24324 | 2036 | 2053 | 38 |
| 586055 | CCCACCTGGGCTCCTG | 24291 | 24306 | 2020 | 2035 | 39 |
| 586056 | CACCCACCTGGGCTCC | 24293 | 24308 | 2022 | 2037 | 40 |
| 586057 | TCCACCCACCTGGGCT | 24295 | 24310 | 2024 | 2039 | 41 |
| 586058 | GGTCCACCCACCTGGG | 24297 | 24312 | 2026 | 2041 | 42 |
| 586059 | TGGGTCCACCCACCTG | 24299 | 24314 | 2028 | 2043 | 43 |

Nucleosides that are underlined are a mismatch to SEQ ID NO: 1;
nucleotides are 100% complementary to SEQ ID NO: 4.

### In Vitro Activity in Patient Fibroblasts

Patient-derived HGPS fibroblasts (Coriell Institute, AG06297; described in Scaffidi and Misteli Nat Cell Biol. 10: 452-459, 2008) were transfected with 100 nM modified oligonucleotide using Lipofectamine®2000 (ThermoFisher) per manufacturer's instructions. After 24 hours, cells were lysed, and mRNA was harvested for analysis.

RT-qPCR was used to analyze RNA levels and quantify relative levels of progerin mRNA and prelamin A mRNA (LMNA). Primer probe sets were designed to only amplify each indicated mRNA variant by selecting binding sites only present in the respective mRNA after splicing events. These primer probe sequences are presented in Table 4 below. Levels of mRNA were normalized with GADPH and normalized to cells that were mock transfected with PBS.

As shown in Tables 5 and 6, below, several modified oligonucleotides complementary to the progerin 5′ splice site are useful to reduce the amount of progerin mRNA (a LMNA transcription product associated with Hutchinson-Gilford progeria syndrome) in HGPS patient fibroblasts.

TABLE 4
Primer Probe Sets

| Variant | | | SEQ ID |
|---|---|---|---|
| detected | | Sequence (5′ to 3′) | NO: |
| prelamin | Forward | CAGCTTCGGGGACAATCTG | 5 |
| A mRNA | Sequence | | |
| | Reverse | GGCATGAGGTGAGGAGGAC | 6 |
| | Sequence | | |
| | Probe | GTCACCCGCTCCTACCTCC | 7 |
| | Sequence | T | |
| progerin | Forward | GCGTCAGGAGCCCTGAGC | 8 |
| mRNA | Sequence | | |
| | Reverse | GACGCAGGAAGCCTCCAC | 9 |
| | Sequence | | |
| | Probe | AGCATCATGTAATCTGGGA | 10 |
| | Sequence | CC | |

TABLE 5
In vitro activity of modified
oligonucleotides complementary to
the 5′-splice site of human progerin

| | | Progerin |
|---|---|---|
| Compound | Sugar Motif | mRNA |
| ID | (5′ to 3′) | (% control) |
| 586000 | kkeekeekeekeekeeke | 91 |
| 586001 | kkeekeekeekeekeeke | 150 |
| 586002 | kkeekeekeekeekeeke | 105 |
| 586003 | kkeekeekeekeekeeke | 100 |
| 586004 | kkeekeekeekeekeeke | 123 |
| 586005 | kkeekeekeekeekeeke | 75 |
| 586006 | kkeekeekeekeekeeke | 133 |
| 586007 | kkeekeekeekeekeeke | 94 |
| 586008 | kkeekeekeekeekeeke | 75 |
| 586009 | kkeekeekeekeekeeke | 188 |
| 586010 | kkeekeekeekeekeeke | 92 |
| 586011 | kkeekeekeekeekeeke | 148 |
| 586012 | kkeekeekeekeekeeke | 97 |
| 586013 | kkeekeekeekeekeeke | 114* |
| 586014 | kkeekeekeekeekeeke | 129* |
| 586015 | kkeekeekeekeekeeke | 120* |
| 586016 | kkeekeekeekeekeeke | 92* |
| 586017 | kkeekeekeekeekeeke | 92 |
| 586018 | kkeekeekeekeekeeke | 99 |
| 586019 | kkeekeekeekeekeeke | 106 |
| 586020 | kkeekeekeekeekeeke | 77 |
| 586021 | kkeekeekeekeekeeke | 78 |
| 586022 | kkeekeekeekeekeeke | 90 |
| 586023 | kkeekeekeekeekeeke | 69 |
| 586024 | kkeekeekeekeekeeke | 99 |
| 586025 | keekeekeekeekeek | 86 |
| 586026 | keekeekeekeekeek | 97 |
| 586027 | keekeekeekeekeek | 68 |
| 586028 | keekeekeekeekeek | 76 |
| 586029 | keekeekeekeekeek | 93 |
| 586030 | kkddkddkddkddkddkk | 72 |
| 586031 | kkddkddkddkddkddkk | 95 |
| 586032 | kkddkddkddkddkddkk | 78 |
| 586033 | kkddkddkddkddkddkk | 67 |
| 586034 | kkddkddkddkddkddkk | 90 |
| 586035 | kkddkddkddkddkddkk | 69 |
| 586036 | kkddkddkddkddkddkk | 100 |
| 586037 | kkddkddkddkddkddkk | 98 |
| 586038 | kkddkddkddkddkddkk | 82 |
| 586039 | kkddkddkddkddkddkk | 101 |
| 586040 | kkddkddkddkddkddkk | 92 |
| 586041 | kkddkddkddkddkddkk | 108 |
| 586042 | kkddkddkddkddkddkk | 76 |
| 586043 | kkddkddkddkddkddkk | 90* |
| 586044 | kkddkddkddkddkddkk | 108* |
| 586045 | kkddkddkddkddkddkk | 94* |
| 586046 | kkddkddkddkddkddkk | 79* |
| 586047 | kkddkddkddkddkddkk | 94 |
| 586048 | kkddkddkddkddkddkk | 89 |
| 586049 | kkddkddkddkddkddkk | 163 |
| 586050 | kkddkddkddkddkddkk | 72 |
| 586051 | kkddkddkddkddkddkk | 78 |
| 586052 | kkddkddkddkddkddkk | 69 |
| 586053 | kkddkddkddkddkddkk | 67 |
| 586054 | kkddkddkddkddkddkk | 110 |
| 586055 | kddkddkddkddkddk | 91 |
| 586056 | kddkddkddkddkddk | 78 |
| 586057 | kddkddkddkddkddk | 83 |
| 586058 | kddkddkddkddkddk | 89 |
| 586059 | kddkddkddkddkddk | 92 |

“k” represents a cEt modified nucleoside comprising a 2′-4′-O—CH(CH3)- bridge.
“e” represents a modified nucleoside comprising a 2′-methoxyethyl modified sugar moiety, and
“d” represents nucleoside comprising a β-D-2′-deoxyribose.
*Values with an asterisk represent the average of two independent experiments

TABLE 6
In vitro activity of modified oligonucleotides complementary
to the 5'-splice site of human progerin

| | Progerin mRNA | Prelamin A mRNA |
|---|---|---|
| Compound ID | (% control) | (% control) |
| 586013 | 108 | 99 |
| 586014 | 145 | 117 |
| 586015 | 124 | 99 |
| 586016 | 116 | 111 |
| 586017 | 98 | 80 |
| 586018 | 98 | 110 |
| 586019 | 113 | 106 |
| 586020 | 83 | 65 |
| 586021 | 78 | 68 |
| 586022 | 97 | 68 |
| 586023 | 68 | 74 |
| 586024 | 106 | 80 |
| 586025 | 92 | 97 |
| 586026 | 98 | 88 |
| 586027 | 63 | 71 |
| 586028 | 81 | 75 |
| 586029 | 90 | 68 |

### Example 2 Effect of Modified Oligonucleotides Complementary to the Human Exon 10 Donor Site of LMNA, In Vitro

Modified oligonucleotides complementary to the exon 10 donor site were designed and tested for their effect on progerin and prelamin A mRNA in vitro. Modified oligonucleotides in the table below are complementary to the exon 10 donor site in human LMNA pre-mRNA (SEQ ID NO: 1) or are complementary to the exon 10 donor site within wild-type human prelamin A mRNA (SEQ ID NO: 2). Each nucleoside of the oligonucleotides in the table below is modified with a 2′-methoxyethyl and each internucleoside linkage is a phosphorothioate internucleoside linkage. Each cytosine residue is a 5-methyl cytosine.

Patient-derived HGPS cells were transfected with 100 nM modified oligonucleotide using Lipofectamine®2000 (ThermoFisher) per manufacturer's instructions. After 24 hours, cells were lysed and mRNA and protein were harvested for analysis.

RT-qPCR was used to analyze mRNA and quantify relative levels of progerin mRNA and prelamin A mRNA, as described in Example 1. As shown in the table below, several modified oligonucleotides complementary to the exon 10 donor site of LMNA are useful to reduce the amount of progerin mRNA (a LMNA transcription product associated with HGPS) in HGPS patient fibroblasts.

TABLE 7
In vitro activity of modified oligonucleotides
complementary to the exon 10 donor site of
human LMNA

| | | SEQ ID | SEQ ID | SEQ ID | SEQ ID | | progerin | prelamin |
|---|---|---|---|---|---|---|---|---|
| | | NO: 1 | NO: 1 | NO: 2 | NO: 2 | SEQ | mRNA | A mRNA |
| Compound | Sequence | start | stop | start | stop | ID | (% | (% |
| ID | (5′ to 3′) | site | site | site | site | NO | Control) | Control) |
| 585180 | GTCCTCAACCAC | 23378 | 23395 | 1851 | 1868 | 44 | 71 | 60 |
| | AGTCAC | | | | | | | |
| 585181 | TCGTCGTCCTCA | 23383 | 23400 | 1856 | 1873 | 45 | 69 | 65 |
| | ACCACA | | | | | | | |
| 585182 | CATCCTCGTCGT | 23388 | 23405 | 1861 | 1878 | 46 | 43 | 40 |
| | CCTCAA | | | | | | | |
| 585183 | ATCCTCATCCTC | 23393 | 23410 | 1866 | 1883 | 47 | 53 | 41 |
| | GTCGTC | | | | | | | |
| 585184 | TCTCCATCCTCA | 23398 | 23415 | 1871 | 1888 | 48 | 56 | 46 |
| | TCCTCG | | | | | | | |
| 585185 | GGTCATCTCCAT | 23403 | 23420 | 1876 | 1893 | 49 | 78 | 73 |
| | CCTCAT | | | | | | | |
| 585186 | GAGCAGGTCATC | 23408 | 23425 | 1881 | 1898 | 50 | 82 | 94 |
| | TCCATC | | | | | | | |
| 585187 | TGATGGAGCAGG | 23413 | 23430 | 1886 | 1903 | 51 | 79 | 78 |
| | TCATCT | | | | | | | |
| 585188 | GGTGGTGATGGA | 23418 | 23435 | 1891 | 1908 | 52 | 87 | 50 |
| | GCAGGT | | | | | | | |
| 585189 | GTGGTGGTGATG | 23420 | 23437 | 1893 | 1910 | 53 | 89 | 83 |
| | GAGCAG | | | | | | | |
| 585190 | ACGTGGTGGTGA | 23422 | 23439 | N/A | N/A | 54 | 49 | 58 |
| | TGGAGC | | | | | | | |
| 585191 | TCACGTGGTGGT | 23424 | 23441 | N/A | N/A | 55 | 33 | 48 |
| | GATGGA | | | | | | | |
| 585192 | ACTCACGTGGTG | 23426 | 23443 | N/A | N/A | 56 | 65 | 78 |
| | GTGATG | | | | | | | |
| 585193 | CCACTCACGTGG | 23428 | 23445 | N/A | N/A | 57 | 61 | 77 |
| | TGGTGA | | | | | | | |
| 585194 | TACCACTCACGT | 23430 | 23447 | N/A | N/A | 58 | 48 | 54 |
| | GGTGGT | | | | | | | |
| 585195 | GCTACCACTCAC | 23432 | 23449 | N/A | N/A | 59 | 49 | 49 |
| | GTGGTG | | | | | | | |
| 585196 | CGGCTACCACTC | 23434 | 23451 | N/A | N/A | 60 | 58 | 54 |
| | ACGTGG | | | | | | | |
| 585197 | GGCGGCTACCAC | 23436 | 23453 | N/A | N/A | 61 | 54 | 59 |
| | TCACGT | | | | | | | |
| 585198 | GCGGCGGCTACC | 23438 | 23455 | N/A | N/A | 62 | 58 | 48 |
| | ACTCAC | | | | | | | |
| 585199 | CAGCGGCGGCTA | 23440 | 23457 | N/A | N/A | 63 | 52 | 70 |
| | CCACTC | | | | | | | |
| 585200 | CTCAGCGGCGGC | 23442 | 23459 | N/A | N/A | 64 | 65 | 56 |
| | TACCAC | | | | | | | |
| 585201 | GCCTCAGCGGCG | 23444 | 23461 | N/A | N/A | 65 | 105 | 70 |
| | GCTACC | | | | | | | |
| 585202 | GCTCGGCCTCAG | 23449 | 23466 | N/A | N/A | 66 | 104 | 157 |
| | CGGCGG | | | | | | | |
| 585203 | TGCAGGCTCGGC | 23454 | 23471 | N/A | N/A | 67 | 86 | 112 |
| | CTCAGC | | | | | | | |
| 585204 | CCCAGTGCAGGC | 23459 | 23476 | N/A | N/A | 68 | 89 | 123 |
| | TCGGCC | | | | | | | |
| 585205 | GTGGCCCCAGTG | 23464 | 23481 | N/A | N/A | 69 | 94 | 137 |
| | CAGGCT | | | | | | | |
| 585206 | GCTGGGTGGCCC | 23469 | 23486 | N/A | N/A | 70 | 103 | 104 |
| | CAGTGC | | | | | | | |
| 585207 | GCCTGGCTGGGT | 23474 | 23491 | N/A | N/A | 71 | 95 | 96 |
| | GGCCCC | | | | | | | |
| 585208 | CCCAGGCCTGGC | 23479 | 23496 | N/A | N/A | 72 | 84 | 94 |
| | TGGGTG | | | | | | | |
| 585209 | CTGCCCCCAGGC | 23484 | 23501 | N/A | N/A | 73 | 49 | 100 |
| | CTGGCT | | | | | | | |

### Example 3 Effect of Combination Treatment in Patient Fibroblasts

Patient-derived HGPS cells were transfected with 100 nM of two different modified oligonucleotides complementary to different sites on LMNA using Lipofectamine®2000 (ThermoFisher) per manufacturer's instructions. After 24 hours, cells were lysed and mRNA was harvested for analysis.

RT-qPCR was used to analyze RNA and quantify relative levels of LMNA isoforms as described in Example 1. The level of lamin C mRNA was also measured using the lamin C primer probe set (forward sequence: ACGGCTCTCATCAACTCCAC(SEQ ID NO: 11), reverse sequence: GCGGCGGCTACCACTCAC (SEQ ID NO: 12), probe sequence: GGTTGAGGACGACGAGGATG(SEQ ID NO:13)). Data are normalized to mock-transfected cells and presented in the table below.

As shown in the Table below, co-administration of modified oligonucleotides is useful to reduce the amount of progerin mRNA (a LMNA transcription product associated with HGPS) in HGPS patient fibroblasts.

TABLE 8
Effect of combination treatment in patient fibroblasts

| | | progerin | prelamin A | lamin C |
|---|---|---|---|---|
| Compound | Compound | mRNA | mRNA | mRNA (% |
| ID 1 | ID 2 | (% control) | (% Control) | Control) |
| 586033 | N/A | 51 | 31 | 154 |
| 586035 | N/A | 75 | 41 | 125 |
| 586038 | N/A | 142 | 65 | 150 |
| 586050 | N/A | 65 | 64 | 210 |
| 586052 | N/A | 58 | 48 | 201 |
| 586053 | N/A | 75 | 54 | 180 |
| 586033 | 586050 | 94 | 55 | 174 |
| 586033 | 586052 | 69 | 38 | 165 |
| 586033 | 586053 | 65 | 43 | 162 |
| 586035 | 586050 | 91 | 77 | 200 |
| 586035 | 586052 | 46 | 38 | 126 |
| 586035 | 586053 | 66 | 48 | 151 |
| 586038 | 586050 | 66 | 48 | 242 |
| 586038 | 586052 | 61 | 33 | 221 |
| 586038 | 586053 | 55 | 43 | 195 |
| 585182 | N/A | 48 | 65 | 22 |
| 585191 | N/A | 63 | 58 | 92 |
| 586033 | 585182 | 72 | 48 | 21 |
| 586033 | 585191 | 54 | 49 | 115 |
| 586035 | 585182 | 68 | 69 | 42 |
| 586035 | 585191 | 75 | 52 | 115 |

### Example 4 Effect of Modified Oligonucleotides Complementary to the Human Progerin 5′ Splice Site, in Vitro

Modified oligonucleotides complementary to the human progerin 5′-splice site were designed and tested for their effect on progerin mRNA, prelamin A mRNA, and lamin C mRNA in vitro. Modified oligonucleotides in the table below are complementary to the wild-type human LMNA pre-mRNA (SEQ ID NO: 1) or the mutant prelamin A mRNA having the HGPS-associated G608G mutation (SEQ ID NO: 4). Each nucleoside of the oligonucleotides in the table below is modified with a 2′-methoxyethyland each internucleoside linkage is a phosphorothioate internucleoside linkage. Each cytosine residue is a 5-methyl cytosine.

Nucleosides that are underlined represent a single nucleoside mismatch to wild-type human LMNA, SEQ ID NO:1 at that position. In the table below, each underlined nucleoside is an adenine, and it is aligned with a cytosine in SEQ ID NO:1. Compounds are 100% complementary to SEQ ID NO: 4.

TABLE 9
Modified oligonucleotides complementary to
the human progerin 5′-splice site

| | | SEQ | SEQ | SEQ | SEQ | |
|---|---|---|---|---|---|---|
| | | ID | ID | ID | ID | |
| | | NO: 1 | NO: 1 | NO: 4 | NO: 4 | SEQ |
| Compound | | start | stop | start | stop | ID |
| ID | Sequence (5′ to 3′) | site | site | site | site | NO: |
| 383790 | GGGTCCACCCACCTGGGCTCCTGAG | 24289 | 24313 | 2018 | 2042 | 74 |
| 637837 | CTGGGCTCCTGAGCCGCTGGCAGAT | 24277 | 24301 | 2006 | 2030 | 75 |
| 637838 | ACCTGGGCTCCTGAGCCGCTGGCAG | 24279 | 24303 | 2008 | 2032 | 76 |
| 637839 | CCACCTGGGCTCCTGAGCCGCTGGC | 24281 | 24305 | 2010 | 2034 | 77 |
| 637840 | ACCCACCTGGGCTCCTGAGCCGCTG | 24283 | 24307 | 2012 | 2036 | 78 |
| 637841 | CCACCCACCTGGGCTCCTGAGCCGC | 24285 | 24309 | 2014 | 2038 | 79 |
| 637842 | GTCCACCCACCTGGGCTCCTGAGCC | 24287 | 24311 | 2016 | 2040 | 80 |
| 637843 | ATGGGTCCACCCACCTGGGCTCCTG | 24291 | 24315 | 2020 | 2044 | 81 |
| 637844 | AGATGGGTCCACCCACCTGGGCTCC | 24293 | 24317 | 2022 | 2046 | 82 |
| 637845 | GGAGATGGGTCCACCCACCTGGGCT | 24295 | 24319 | 2024 | 2048 | 83 |
| 637846 | GAGGAGATGGGTCCACCCACCTGGG | 24297 | 24321 | 2026 | 2050 | 84 |
| 637847 | CAGAGGAGATGGGTCCACCCACCTG | 24299 | 24323 | 2028 | 2052 | 85 |
| 637848 | GCCAGAGGAGATGGGTCCACCCACC | 24301 | 24325 | 2030 | 2054 | 86 |
| 637849 | GAGCCAGAGGAGATGGGTCCACCCA | 24303 | 24327 | 2032 | 2056 | 87 |
| 637850 | AAGAGCCAGAGGAGATGGGTCCACC | 24305 | 24329 | 2034 | 2058 | 88 |
| 637851 | AGAAGAGCCAGAGGAGATGGGTCCA | 24307 | 24331 | 2036 | 2060 | 89 |
| 637852 | GCAGAAGAGCCAGAGGAGATGGGTC | 24309 | 24333 | 2038 | 2062 | 90 |

Nucleosides that are underlined are a mismatch to SEQ ID NO: 1.

Patient-derived HGPS cells were transfected with 100 nM modified oligonucleotide using Lipofectamine®2000 (ThermoFisher) per manufacturer's instructions. After 24 hours, cells were lysed and mRNA and protein were harvested for analysis.

RT-qPCR was used to analyze mRNA and quantify relative levels of LMNA mRNA isoforms as described in Examples 1 and 3. As shown in the Table below, several modified oligonucleotides complementary to the exon 10 donor site of LMNA are useful to reduce the amount of progerin mRNA (a LMNA transcription product associated with HGPS) in HGPS patient fibroblasts.

TABLE 10
Activity of modified oligonucleotides complementary
to the 5'-splice site of human progerin

| | Progerin | Wild type prelamin | Lamin C |
|---|---|---|---|
| Compound | mRNA | A mRNA | mRNA |
| ID | (% control) | (% control) | (% control) |
| 383790 | 181 | 91 | 182 |
| 637837 | 71 | 66 | 153 |
| 637838 | 137 | 81 | 113 |
| 637839 | 133 | 70 | 89 |
| 637840 | 122 | 48 | 91 |
| 637841 | 65 | 39 | 87 |
| 637842 | 118 | 33 | 135 |
| 637843 | 109 | 38 | 103 |
| 637844 | 269 | 147 | 141 |
| 637845 | 67 | 59 | 135 |
| 637846 | 104 | 68 | 111 |
| 637847 | 121 | 37 | 119 |
| 637848 | 40 | 27 | 136 |
| 637849 | 46 | 25 | 167 |
| 637850 | 83 | 28 | 112 |
| 637851 | 141 | 155 | 199 |
| 637852 | 95 | 19 | 50 |

### Example 5 Modified Oligonucleotides Complementary to Alternatively Spliced LMNA Isoforms

Modified oligonucleotides complementary to several LMNA isoforms were designed and tested for their effect on progerin mRNA, prelamin A mRNA, and lamin C mRNA in vitro. Modified oligonucleotides in the table below are complementary to the wild-type prelamin A human mRNA (SEQ ID NO: 2) or the progerin mRNA (SEQ ID NO: 3). The compounds in the tables below have (1) a 5-10-5 MOE gapmer motif, consisting of 5 linked MOE modified nucleosides in the 5′-wing, 10 linked β-D-2′-deoxyribonucleosides in the gap, and 5 linked MOE nucleosides in the 3′-wing (Table 11); (2) a 3-10-3 cEt gapmer motif, consisting of 3 linked cEt modified nucleosides in the 5′-wing, 10 linked β-D-2′-deoxyribonucleosides in the gap, and 3 linked cEt nucleosides in the 3′-wing (Table 12); or (3) a mixed sugar motif kk-d(8)-kekeke, where k represents a cEt modified nucleoside, e represents a MOE modified nucleoside, and d represents a β-D-2′-deoxyribonucleoside (Table 13). Each internucleoside linkage in the modified oligonucleotides below is a phosphorothioate internucleoside linkage. Each cytosine residue is a 5-methyl cytosine.

TABLE 11
5-10-5 MOE gapmer modified oligonucleotides
complementary to progerin

| | | SEQ ID | SEQ ID | SEQ ID | SEQ ID | |
|---|---|---|---|---|---|---|
| | | NO: 2 | NO: 2 | NO: 3 | NO: 3 | SEQ |
| Compound | | start | stop | start | stop | ID |
| ID | Sequence (5′ to 3′) | site | site | site | site | NO: |
| 638291 | GGGGCTCTGGGCTCCTGAGC | N/A | N/A | 2054 | 2073 | 91 |
| 638292 | GGGGGCTCTGGGCTCCTGAG | N/A | N/A | 2055 | 2074 | 92 |
| 638293 | TGGGGGCTCTGGGCTCCTGA | N/A | N/A | 2056 | 2075 | 93 |
| 638294 | AGTTCTGGGGGCTCTGGGCT | N/A | N/A | 2061 | 2080 | 94 |
| 638295 | CAGTTCTGGGGGCTCTGGGC | N/A | N/A | 2062 | 2081 | 95 |
| 638296 | GCAGTTCTGGGGGCTCTGGG | 2176 | 2195 | 2063 | 2082 | 96 |
| 638297 | GCTGCAGTTCTGGGGGCTCT | 2179 | 2198 | 2066 | 2085 | 97 |
| 638298 | TGCTGCAGTTCTGGGGGCTC | 2180 | 2199 | 2067 | 2086 | 98 |
| 638299 | CTGGGCTCCTGAGCCGCTGG | 2011 | 2030 | 2048 | 2067 | 99 |
| 638300 | CTCTGGGCTCCTGAGCCGCT | N/A | N/A | 2050 | 2069 | 100 |
| 638301 | GCTCTGGGCTCCTGAGCCGC | N/A | N/A | 2051 | 2070 | 101 |
| 358688 | TCTGGGGGCTCTGGGCTCCT | N/A | N/A | 2058 | 2077 | 102 |
| 366687 | GGGCTCTGGGCTCCTGAGCC | N/A | N/A | 2053 | 2072 | 103 |
| 366791 | CTGCAGTTCTGGGGGCTCTG | N/A | N/A | 2065 | 2084 | 104 |
| 366822 | TCTGGGCTCCTGAGCCGCTG | N/A | N/A | 2049 | 2068 | 105 |
| 366823 | GGCTCTGGGCTCCTGAGCCG | N/A | N/A | 2052 | 2071 | 106 |
| 366824 | CTGGGGGCTCTGGGCTCCTG | N/A | N/A | 2057 | 2076 | 107 |
| 366825 | TTCTGGGGGCTCTGGGCTCC | N/A | N/A | 2059 | 2078 | 108 |
| 366826 | GTTCTGGGGGCTCTGGGCTC | N/A | N/A | 2060 | 2079 | 109 |
| 366827 | TGCAGTTCTGGGGGCTCTGG | N/A | N/A | 2064 | 2083 | 110 |
| 366828 | ATGCTGCAGTTCTGGGGGCT | N/A | N/A | 2068 | 2087 | 111 |
| 358688 | TCTGGGGGCTCTGGGCTCCT | N/A | N/A | 2058 | 2077 | 102 |

TABLE 12
3-10-3 cEt gapmer modified oligonucleotides
complementary to progerin

| | | SEQ ID | SEQ ID | SEQ ID | SEQ ID | |
|---|---|---|---|---|---|---|
| | | NO: 2 | NO: 2 | NO: 3 | NO: 3 | SEQ |
| Compound | Sequence | start | stop | start | stop | ID |
| ID | (5′ to 3′) | site | site | site | site | NO: |
| 638257 | CTGGGCTCCTGAGCCG | 2015 | 2030 | 2052 | 2067 | 112 |
| 638258 | TCTGGGCTCCTGAGCC | 2016 | 2031 | 2053 | 2068 | 113 |
| 638259 | CTCTGGGCTCCTGAGC | N/A | N/A | 2054 | 2069 | 114 |
| 638260 | GCTCTGGGCTCCTGAG | N/A | N/A | 2055 | 2070 | 115 |
| 638261 | GGCTCTGGGCTCCTGA | N/A | N/A | 2056 | 2071 | 116 |
| 638262 | GGGCTCTGGGCTCCTG | N/A | N/A | 2057 | 2072 | 117 |
| 638263 | GGGGCTCTGGGCTCCT | N/A | N/A | 2058 | 2073 | 118 |
| 638264 | GGGGGCTCTGGGCTCC | N/A | N/A | 2059 | 2074 | 119 |
| 638265 | TGGGGGCTCTGGGCTC | N/A | N/A | 2060 | 2075 | 120 |
| 638266 | CTGGGGGCTCTGGGCT | N/A | N/A | 2061 | 2076 | 121 |
| 638267 | TCTGGGGGCTCTGGGC | N/A | N/A | 2062 | 2077 | 122 |
| 638268 | TTCTGGGGGCTCTGGG | 2176 | 2191 | 2063 | 2078 | 123 |
| 638269 | GTTCTGGGGGCTCTGG | 2177 | 2192 | 2064 | 2079 | 124 |
| 638270 | AGTTCTGGGGGCTCTG | 2178 | 2193 | 2065 | 2080 | 125 |
| 638271 | CAGTTCTGGGGGCTCT | 2179 | 2194 | 2066 | 2081 | 126 |
| 638272 | GCAGTTCTGGGGGCTC | 2180 | 2195 | 2067 | 2082 | 127 |
| 638273 | TGCAGTTCTGGGGGCT | 2181 | 2196 | 2068 | 2083 | 128 |

TABLE 13
modified oligonucleotides with a kk-d8-kekeke
sugar motif (5′ to 3′) complementary to progerin

| | | SEQ ID | SEQ ID | SEQ ID | SEQ ID | SEQ |
|---|---|---|---|---|---|---|
| Compound | Sequence | NO: 2 | NO: 2 | NO: 3 | NO: 3 | ID |
| ID | (5′ to 3′) | start site | stop site | start site | stop site | NO: |
| 638274 | CTGGGCTCCTGAGCCG | 2015 | 2030 | 2052 | 2067 | 112 |
| 638275 | TCTGGGCTCCTGAGCC | N/A | N/A | 2053 | 2068 | 113 |
| 638276 | CTCTGGGCTCCTGAGC | N/A | N/A | 2054 | 2069 | 114 |
| 638277 | GCTCTGGGCTCCTGAG | N/A | N/A | 2055 | 2070 | 115 |
| 638278 | GGCTCTGGGCTCCTGA | N/A | N/A | 2056 | 2071 | 116 |
| 638279 | GGGCTCTGGGCTCCTG | N/A | N/A | 2057 | 2072 | 117 |
| 638280 | GGGGCTCTGGGCTCCT | N/A | N/A | 2058 | 2073 | 118 |
| 638281 | GGGGGCTCTGGGCTCC | N/A | N/A | 2059 | 2074 | 119 |
| 638282 | TGGGGGCTCTGGGCTC | N/A | N/A | 2060 | 2075 | 120 |
| 638283 | CTGGGGGCTCTGGGCT | N/A | N/A | 2061 | 2076 | 121 |
| 638284 | TCTGGGGGCTCTGGGC | N/A | N/A | 2062 | 2077 | 122 |
| 638285 | TTCTGGGGGCTCTGGG | 2176 | 2191 | 2063 | 2078 | 123 |
| 638286 | GTTCTGGGGGCTCTGG | 2177 | 2192 | 2064 | 2079 | 124 |
| 638287 | AGTTCTGGGGGCTCTG | 2178 | 2193 | 2065 | 2080 | 125 |
| 638288 | CAGTTCTGGGGGCTCT | 2179 | 2194 | 2066 | 2081 | 126 |
| 638289 | GCAGTTCTGGGGGCTC | 2180 | 2195 | 2067 | 2082 | 127 |
| 638290 | TGCAGTTCTGGGGGCT | 2181 | 2196 | 2068 | 2083 | 128 |

Patient-derived HGPS cells were transfected with 100 nM modified oligonucleotide using Lipofectamine®2000 (ThermoFisher) per manufacturer's instructions. After 24 hours, cells were lysed and mRNA was harvested for analysis.

RT-qPCR was used to analyze mRNA and quantify relative levels of LMNA isoforms as described in Examples 1 and 3. As shown in the Table below, several modified oligonucleotides are useful to reduce the amount of progerin mRNA (a LMNA transcription product associated with HGPS) in HGPS patient fibroblasts.

TABLE 14
Activity of modified oligonucleotides complementary to
human progerin mRNA or pre-mRNA

| | progerin | prelamin A | Lamin C |
|---|---|---|---|
| Compound | mRNA | mRNA (% | mRNA |
| ID | (% control) | control) | (% control) |
| 638291 | 83 | 64 | 94 |
| 638292 | 58 | 52 | 112 |
| 638293 | 68 | 63 | 115 |
| 638294 | 55 | 36 | 137 |
| 638295 | 47 | 26 | 104 |
| 638296 | 35 | 31 | 85 |
| 638297 | 28 | 29 | 116 |
| 638298 | 68 | 47 | 127 |
| 638299 | 25 | 29 | 94 |
| 638300 | 22 | 13 | 38 |
| 638301 | 24 | 8 | 81 |
| 358688 | 50 | 63 | 114 |
| 366687 | 51 | 61 | 121 |
| 366791 | 41 | 50 | 119 |
| 366822 | 25 | 22 | 45 |
| 366823 | 53 | 58 | 101 |
| 366824 | 54 | 49 | 106 |
| 366825 | 51 | 53 | 119 |
| 366826 | 48 | 48 | 134 |
| 366827 | 48 | 42 | 118 |
| 366828 | 46 | 41 | 104 |
| 638257 | 66 | 49 | 60 |
| 638258 | 61 | 37 | 52 |
| 638259 | 63 | 48 | 77 |
| 638260 | 69 | 53 | 61 |
| 638261 | 68 | 44 | 86 |
| 638262 | 95 | 61 | 78 |
| 638263 | 94 | 43 | 53 |
| 638264 | 83 | 62 | 72 |
| 638265 | 90 | 55 | 73 |
| 638266 | 82 | 28 | 70 |
| 638267 | 68 | 46 | 80 |
| 638268 | 82 | 54 | 78 |
| 638269 | 77 | 37 | 72 |
| 638270 | 110 | 59 | 95 |
| 638271 | 101 | 72 | 92 |
| 638272 | 119 | 73 | 79 |
| 638273 | 100 | 85 | 128 |
| 638274 | 96 | 99 | 106 |
| 638275 | 103 | 134 | 131 |
| 638276 | 104 | 121 | 89 |
| 638277 | 92 | 108 | 106 |
| 638278 | 71 | 82 | 87 |
| 638279 | 97 | 104 | 97 |
| 638280 | 110 | 128 | 102 |
| 638281 | 110 | 115 | 112 |
| 638282 | 88 | 137 | 92 |
| 638283 | 103 | 140 | 84 |
| 638284 | 106 | 119 | 113 |
| 638285 | 11 | 106 | 99 |
| 638286 | 91 | 70 | 75 |
| 638287 | 102 | 147 | 120 |
| 638288 | 89 | 126 | 73 |
| 638289 | 144 | 120 | 153 |
| 638290 | 197 | 158 | 187 |

5-10-5 MOE or 3-10-3 cEt modified oligonucleotides can modulate the splicing of LMNA in HGPS patient fibroblasts.

### Example 6 Modified Oligonucleotides Complementary to Prelamin A or Progerin mRNA

Modified oligonucleotides complementary to several LMNA isoforms were designed and tested for their effect on progerin mRNA, prelamin A mRNA, and lamin C mRNA in vitro. Modified oligonucleotides in the table below are complementary to the complementary to the prelamin A human mRNA (SEQ ID NO: 2) or progerin mRNA (SEQ ID NO: 3). Each nucleoside of the oligonucleotides in the table below is either (1) modified with 2′-methoxyethyl; or (2) comprises 2′-methoxyethyl nucleosides and β-D-2′-deoxyribonucleosides having the motif (5′-3′) of edededededededededee, as indicated in the table below. Each internucleoside linkage is a phosphorthioate internucleoside linkage. Each cytosine is a 5-methyl cytosine. As shown in Table 16, below, several modified oligonucleotides are useful to reduce the amount of progerin mRNA (a LMNA transcription product associated with HGPS) in HGPS patient fibroblasts.

TABLE 15
Modified Oligonucleotides

| | | | SEQ | SEQ | SEQ | SEQ | |
|---|---|---|---|---|---|---|---|
| | | | ID | ID | ID | ID | |
| | | | NO: 2 | NO: 2 | NO: 3 | NO: 3 | SEQ |
| Compound | Sequence | Sugar Motif | start | stop | start | stop | ID |
| ID | (5′ to 3′) | (5′ to 3′) | site | site | site | site | NO: |
| 796553 | CAGTTCTGGGG | Uniform MOE | N/A | N/A | 2062 | 2081 | 95 |
| | GCTCTGGGC | | | | | | |
| 796554 | GCAGTTCTGGG | Uniform MOE | 2176 | 2195 | 2063 | 2082 | 96 |
| | GGCTCTGGG | | | | | | |
| 796555 | GCTGCAGTTCT | Uniform MOE | 2179 | 2198 | 2066 | 2085 | 97 |
| | GGGGGCTCT | | | | | | |
| 796556 | CTGGGCTCCTG | Uniform MOE | 2011 | 2030 | 2048 | 2067 | 99 |
| | AGCCGCTGG | | | | | | |
| 796557 | CTCTGGGCTCC | Uniform MOE | N/A | N/A | 2050 | 2069 | 100 |
| | TGAGCCGCT | | | | | | |
| 796558 | GCTCTGGGCTC | Uniform MOE | N/A | N/A | 2051 | 2070 | 101 |
| | CTGAGCCGC | | | | | | |
| 796559 | TCTGGGCTCCT | Uniform MOE | N/A | N/A | 2049 | 2068 | 105 |
| | GAGCCGCTG | | | | | | |
| 796560 | CAGTTCTGGGG | edededededededededee | N/A | N/A | 2062 | 2081 | 95 |
| | GCTCTGGGC | | | | | | |
| 796561 | GCAGTTCTGGG | edededededededededee | 2176 | 2195 | 2063 | 2082 | 96 |
| | GGCTCTGGG | | | | | | |
| 796562 | GCTGCAGTTCT | edededededededededee | 2179 | 2198 | 2066 | 2085 | 97 |
| | GGGGGCTCT | | | | | | |
| 796563 | CTGGGCTCCTG | edededededededededee | 2011 | 2030 | 2048 | 2067 | 99 |
| | AGCCGCTGG | | | | | | |
| 796564 | CTCTGGGCTCC | edededededededededee | N/A | N/A | 2050 | 2069 | 100 |
| | TGAGCCGCT | | | | | | |
| 796565 | GCTCTGGGCTC | edededededededededee | N/A | N/A | 2051 | 2070 | 101 |
| | CTGAGCCGC | | | | | | |
| 796566 | TCTGGGCTCCT | edededededededededee | N/A | N/A | 2049 | 2068 | 105 |
| | GAGCCGCTG | | | | | | |

e represents a modified nucleoside comprising a 2′-methoxyethyl modified sugar moiety;
d represents a nucleoside comprising a β-D-2′-deoxyribose sugar moiety.

TABLE 16
Activity of modified oligonucleotides complementary
to various isoforms of LMNA

| | | prelamin | |
|---|---|---|---|
| | progerin | A | Lamin C |
| | mRNA | mRNA (% | mRNA |
| Compound ID | (% control) | control) | (% control) |
| 796553 | 3 | 1 | 135 |
| 796554 | 3 | 1 | 125 |
| 796555 | 4 | 2 | 120 |
| 796556 | 21 | 47 | 67 |
| 796557 | 9 | 36 | 56 |
| 796558 | 10 | 45 | 58 |
| 796559 | 22 | 54 | 73 |
| 796560 | 9 | 28 | 134 |
| 796561 | 15 | 29 | 138 |
| 796562 | 16 | 37 | 142 |
| 796563 | 15 | 22 | 239 |
| 796564 | 14 | 21 | 187 |
| 796565 | 8 | 33 | 187 |
| 796566 | 13 | 25 | 211 |

### Example 7 Design of Modified Oligonucleotides Complementary to the 5′-Splice Site of Progerin mRNA

Modified oligonucleotides complementary to the 5′-splice site of progerin mRNA were designed. Modified oligonucleotides in the table below are complementary to the wild-type human genomic sequence of LMNA (SEQ ID NO: 1) or the mutant prelamin A mRNA having the HGPS-associated G608G mutation (SEQ ID NO: 4). The oligonucleotides comprise 2′-4′-constrained ethyl (cEt) nucleosides and deoxyribonucleosides as indicated in the table below, and each internucleoside linkage is a phosphorothioate internucleoside linkage. Each cytosine residue is a 5-methyl cytosine. Sugar motif of the modified oligonucleotides is indicated in Table 18. Nucleosides that are underlined represent a single nucleoside mismatch to wild-type human genomic sequence, SEQ ID NO:1.

TABLE 17
Modified Oligonucleotides

| | | SEQ | SEQ | SEQ | SEQ | |
|---|---|---|---|---|---|---|
| | | ID | ID | ID | ID | |
| | | NO: 1 | NO: 1 | NO: 4 | NO: 4 | SEQ |
| Compound | Sequence | start | stop | start | stop | ID |
| ID | (5′ to 3′) | site | site | site | site | NO: |
| 637853 | ATGGGTCCACCCACCT | 24300 | 24315 | 2029 | 2044 | 129 |
| 637856 | GAGATGGGTCCACCCA | 24303 | 24318 | 2032 | 2047 | 130 |
| 637857 | GGAGATGGGTCCACCC | 24304 | 24319 | 2033 | 2048 | 131 |

TABLE 18
Chemistry Motifs

| Compound | Chemistry Motif |
|---|---|
| ID | (5′ to 3′) |
| 637853 | kddkddkddkddkddk |
| 637856 | kddkddkddkddkddk |
| 637857 | kddkddkddkddkddk |

### Example 8 Protein Levels in Cells Treated with Modified Oligonucleotides

Modified oligonucleotides described above were designed and tested for their effect on progerin mRNA, prelamin A mRNA, and lamin C mRNA in vitro. HGPS cells were transfected with 100 nM of modified oligonucleotide using Lipofectamine®2000. Cells were harvested and protein was analyzed 48 hours after transfection by western blot western blot with the antibody ab40567 (Abcam), which detects an epitope that is common to lamin A, lamin C, and progerin. Levels of protein were normalized to beta-actin and are reported relative to levels in cells undergoing mock transfection (100%). Modified oligonucleotides were useful to reduce progerin and lamin A protein levels.

TABLE 19
Reduction of protein levels in HGPS fibroblasts

| Compound | Progerin | Lamin A (% | Lamin C |
|---|---|---|---|
| ID | (% control) | control) | (% control) |
| 638296 | 6.5 | 5.2 | 86 |
| 638297 | 7.2 | 5.2 | 85 |
| 638299 | 13 | 9.0 | 42 |
| 638300 | 11 | 9.4 | 37 |
| 638301 | 7.3 | 5.3 | 30 |
| 366822 | 20 | 11 | 47 |
| 637853 | 38 | 16 | 176 |
| 637856 | 66 | 29 | 200 |
| 637857 | 33 | 15 | 166 |

### Example 9 Effect of Modified Oligonucleotides Complementary to Human LMNA

Modified oligonucleotides complementary to several LMNA isoforms were designed and tested for their effect on progerin mRNA, prelamin A mRNA, and lamin C mRNA in vitro. Modified oligonucleotides in the table below are complementary to human LMNA pre-mRNA (SEQ ID NO: 1) or are complementary to progerin mRNA (SEQ ID NO: 3). The oligonucleotides comprise 2′-4′-constrained ethyl (cEt) nucleosides, 2′-methoxyethyl nucleosides, and deoxyribonucleosides as indicated in the table below, and all compounds have a full phosphorothioate backbone. Each cytosine residue is a 5-methyl cytosine. The chemical modifications are indicated in the chemistry notation column according the legend following the table.

TABLE 20
Modified Oligonucleotides

| | | SEQ ID | SEQ ID | SEQ ID | SEQ ID | SEQ ID | SEQ ID | |
|---|---|---|---|---|---|---|---|---|
| | | NO: 1 | NO: 1 | NO: 2 | NO: 2 | NO: 3 | NO: 3 | SEQ |
| Compound | Sequence and Chemistry | start | stop | start | stop | start | stop | ID |
| ID | Notation (5′ to 3′) | site | site | site | site | site | site | NO |
| 796553 | ^{m}CₑₛAₑₛGₑₛTₑₛTₑₛ^{m}CₑₛTₑₛGₑₛGₑₛGₑₛ | n/a | n/a | N/A | N/A | 2025 | 2044 | 95 |
| | Gₑₛ^{m}CₑₛTₑₛ^{m}CₑₛTₑₛGₑₛGₑₛGₑₛ^{m}Cₑ | | | | | | | |
| 847115 | GₑₛGₑₛGₑₛ^{m}CₑₛTₑₛ^{m}CₑₛTₑₛAₑₛAₑₛGₑₛ | 24759 | 24778 | N/A | N/A | N/A | N/A | 132 |
| | AₑₛGₑₛAₑₛGₑₛAₑₛAₑₛAₑₛAₑₛ^{m}CₑₛAₑ | | | | | | | |
| 847118 | Gₖₛ^{m}C_{ds}T_{ds}^{m}CₖₛT_{ds}A_{ds}AₖₛG_{ds}A_{ds}Gₖₛ | 24761 | 24776 | N/A | N/A | N/A | N/A | 133 |
| | A_{ds}G_{ds}AₖₛA_{ds}A_{ds}Aₖ | | | | | | | |
| 847119 | GₖₛG_{ds}^{m}C_{ds}Tₖₛ^{m}C_{ds}T_{ds}AₖₛA_{ds}G_{ds}Aₖₛ | 24762 | 24777 | N/A | N/A | N/A | N/A | 134 |
| | G_{ds}A_{ds}GₖₛA_{ds}A_{ds}Aₖ | | | | | | | |
| 847120 | GₖₛG_{ds}G_{ds}^{m}CₖₛT_{ds}^{m}C_{ds}TₖₛA_{ds}A_{ds}Gₖₛ | 24763 | 24778 | N/A | N/A | N/A | N/A | 135 |
| | A_{ds}G_{ds}AₖₛG_{ds}A_{ds}Aₖ | | | | | | | |
| 847121 | GₖₛI_{ds}G_{ds}Gₖₛ^{m}C_{ds}T_{ds}^{m}CₖₛT_{ds}A_{ds}Aₖₛ | 24764 | 24779 | N/A | N/A | N/A | N/A | 136 |
| | G_{ds}A_{ds}GₖₛA_{ds}G_{ds}Aₖ | | | | | | | |
| 847122 | GₖₛG_{ds}I_{ds}GₖₛI_{ds}^{m}C_{ds}Tₖₛ^{m}C_{ds}T_{ds}Aₖₛ | 24765 | 24780 | N/A | N/A | N/A | N/A | 137 |
| | A_{ds}G_{ds}AₖₛG_{ds}A_{ds}Gₖ | | | | | | | |
| 847123 | TₖₛG_{ds}I_{ds}GₖₛI_{ds}G_{ds}^{m}CₖₛT_{ds}^{m}C_{ds}Tₖₛ | 24766 | 24781 | N/A | N/A | N/A | N/A | 138 |
| | A_{ds}A_{ds}GₖₛA_{ds}G_{ds}Aₖ | | | | | | | |
| 847124 | ^{m}CₖₛT_{ds}I_{ds}GₖₛI_{ds}G_{ds}Gₖₛ^{m}_{ds}T_{ds}^{m}Cₖₛ | 24767 | 24782 | N/A | N/A | N/A | N/A | 139 |
| | T_{ds}A_{ds}AₖₛG_{ds}A_{ds}Gₖ | | | | | | | |
| 847125 | Tₖₛ^{m}C_{ds}T_{ds}GₖₛI_{ds}G_{ds}GₖₛI_{ds}^{m}C_{ds}Tₖₛ | 24768 | 24783 | N/A | N/A | N/A | N/A | 140 |
| | ^{m}C_{ds}T_{ds}AₖₛA_{ds}G_{ds}Aₖ | | | | | | | |
| 847126 | TₖₛT_{ds}^{m}C_{ds}TₖₛG_{ds}I_{ds}GₖₛI_{ds}G_{ds}^{m}Cₖₛ | 24769 | 24784 | N/A | N/A | N/A | N/A | 141 |
| | T_{ds}^{m}C_{ds}TₖₛA_{ds}A_{ds}Gₖ | | | | | | | |
| 847127 | GₖₛT_{ds}T_{ds}^{m}CₖₛT_{ds}I_{ds}GₖₛI_{ds}G_{ds}Gₖₛ | 24770 | 24785 | N/A | N/A | N/A | N/A | 142 |
| | ^{m}C_{ds}T_{ds}^{m}CₖₛT_{ds}A_{ds}Aₖ | | | | | | | |
| 847129 | ^{m}CₖₛA_{ds}G_{ds}TₖₛT_{ds}^{m}C_{ds}TₖₛG_{ds}I_{ds}Gₖₛ | 24772 | 24787 | 2179 | 2194 | 2029 | 2044 | 143 |
| | I_{ds}G_{ds}^{m}CₖₛT_{ds}^{m}C_{ds}Tₖ | | | | | | | |
| 847130 | Gₑₛ^{m}CₑₛAₑₛGₑₛTₑₛT_{ds}^{m}C_{ds}T_{ds}G_{ds}I_{ds} | 24769 | 24788 | N/A | N/A | N/A | N/A | 144 |
| | G_{ds}I_{ds}G_{ds}^{m}C_{ds}T_{ds}^{m}CₑₛTₑₛAₑₛAₑₛGₑ | | | | | | | |
| 847131 | ^{m}CₑₛTₑₛGₑₛ^{m}CₑₛAₑₛG_{ds}T_{ds}T_{ds}^{m}C_{ds} | 24771 | 24790 | N/A | N/A | N/A | N/A | 145 |
| | T_{ds}G_{ds}I_{ds}G_{ds}I_{ds}G_{ds}CₑₛTₑₛ^{m}CₑₛTₑₛ | | | | | | | |
| | Aₑ | | | | | | | |
| 847134 | TₑₛGₑₛI_{ds}GₑₛI_{ds}Gₑₛ^{m}CₑₛTₑₛ^{m}CₑₛTₑₛ | 24762 | 24781 | N/A | N/A | N/A | N/A | 146 |
| | AₑₛAₑₛGₑₛAₑₛGₑₛAₑₛGₑₛAₑₛAₑₛAₑ | | | | | | | |
| 847135 | ^{m}CₑₛTₑₛGₑₛI_{ds}GₑₛI_{ds}Gₑₛ^{m}CₑₛTₑₛ | 24763 | 24782 | N/A | N/A | N/A | N/A | 147 |
| | ^{m}CₑₛTₑₛAₑₛAₑₛGₑₛAₑₛGₑₛAₑₛGₑₛ | | | | | | | |
| | AₑₛAₑ | | | | | | | |
| 847136 | Tₑₛ^{m}CₑₛTₑₛGₑₛI_{ds}GₑₛI_{ds}Gₑₛ^{m}CₑₛTₑₛ | 24764 | 24783 | N/A | N/A | N/A | N/A | 148 |
| | ^{m}CₑₛTₑₛAₑₛAₑₛGₑₛAₑₛGₑₛAₑₛGₑₛAₑ | | | | | | | |
| 847137 | TₑₛTₑₛ^{m}CₑₛTₑₛGₑₛI_{ds}GₑₛI_{ds}Gₑₛ^{m}Cₑₛ | 24765 | 24784 | N/A | N/A | N/A | N/A | 149 |
| | Tₑₛ^{m}CₑₛTₑₛAₑₛAₑₛGₑₛAₑₛGₑₛAₑₛGₑ | | | | | | | |
| 847142 | TₑₛGₑₛ^{m}CₑₛAₑₛGₑₛTₑₛTₑₛ^{m}CₑₛTₑₛGₑₛ | 24770 | 24789 | N/A | N/A | N/A | N/A | 150 |
| | I_{ds}GₑₛI_{dS}Gₑₛ^{m}CₑₛTₑₛ^{m}CₑₛTₑₛAₑₛAₑ | | | | | | | |
| 847143 | ^{m}CₑₛTₑₛGₑₛ^{m}CₑₛAₑₛGₑₛTₑₛTₑₛ^{m}Cₑₛ | 24771 | 24790 | N/A | N/A | N/A | N/A | 145 |
| | TₑₛGₑₛI_{ds}GₑₛI_{ds}Gₑₛ^{m}CₑₛTₑₛ^{m}Cₑₛ | | | | | | | |
| | TₑₛAₑ | | | | | | | |
| 847144 | Gₑₛ^{m}CₑₛTₑₛGₑₛ^{m}CₑₛAₑₛGₑₛTₑₛTₑₛ | 24772 | 24791 | 2179 | 2198 | 2029 | 2048 | 151 |
| | ^{m}CₑₛTₑₛGₑₛI_{ds}GₑₛI_{ds}Gₑₛ^{m}CₑₛTₑₛ | | | | | | | |
| | ^{m}CₑₛTₑ | | | | | | | |
| 847935 | GₑₛTₑₛTₑₛ^{m}CₑₛTₑₛGₑₛI_{ds}GₑₛI_{ds}Gₑₛ | 24766 | 24785 | N/A | N/A | N/A | N/A | 152 |
| | ^{m}CₑₛTₑₛ^{m}CₑₛTₑₛAₑₛAₑₛGₑₛAₑₛGₑₛAₑ | | | | | | | |
| 847936 | AₑₛGₑₛTₑₛTₑₛ^{m}CₑₛTₑₛGₑₛI_{ds}GₑₛI_{ds} | 24767 | 24786 | N/A | N/A | N/A | N/A | 153 |
| | Gₑₛ^{m}CₑₛTₑₛ^{m}CₑₛTₑₛAₑₛAₑₛGₑₛAₑₛGₑ | | | | | | | |
| 847938 | Gₑₛ^{m}CₑₛAₑₛGₑₛTₑₛTₑₛ^{m}CₑₛTₑₛGₑₛI_{ds} | 24769 | 24788 | N/A | N/A | N/A | N/A | 144 |
| | GₑₛI_{ds}Gₑₛ^{m}CₑₛTₑₛ^{m}CₑₛTₑₛAₑₛAₑₛGₑ | | | | | | | |

A subscript “e” indicates a nucleoside comprising a 2′-methoxyethyl modified sugar moiety;
a subscript “k” indicates a nucleoside comprising a 2′-constrained ethyl modified sugar moiety;
a subscript “s” indicates a phosphorothioate internucleoside linkage;
a subscript “d” indicates a nucleoside comprising a β-D-2′-deoxyribose sugar moiety;
“I” indicates a nucleoside comprising a hypoxanthine nucleobase; and
“^{m}C” indicates 5-methylCytosine.

HGPS skin fibroblasts were plated at 100,000 cells per well in a 6 well plate for 24 hours prior to transfection with modified oligonucleotide. Modified oligonucleotides were added at 100 nM and transfected with Lipofectamine®2000 (ThermoFisher) per manufacturer's instructions. After 48 hours, cells were lysed and mRNA and protein were harvested for analysis.

RT-qPCR was used to analyze mRNA and quantify relative levels of LMNA isoforms as described in Examples 1 and 3. As shown in the table below, several modified oligonucleotides are useful to reduce the amount of progerin mRNA (a LMNA transcription product associated with HGPS) in HGPS patient fibroblasts.

TABLE 21
In vitro activity of modified oligonucleotides

| Compound | prelamin A mRNA | progerin mRNA | lamin C mRNA |
|---|---|---|---|
| ID | (% control) | (% control) | (% control) |
| 847115 | 45 | 90 | 143 |
| 847118 | 112 | 54 | 111 |
| 847119 | 28 | 19 | 101 |
| 847120 | 77 | 30 | 104 |
| 847121 | 66 | 26 | 93 |
| 847122 | 105 | 58 | 117 |
| 847123 | 80 | 36 | 87 |
| 847124 | 57 | 31 | 100 |
| 847125 | 45 | 22 | 90 |
| 847126 | 84 | 52 | 100 |
| 847127 | 49 | 31 | 120 |
| 847129 | 14 | 25 | 120 |
| 847130 | 32 | 30 | 85 |
| 847131 | 11 | 30 | 108 |
| 847134 | 43 | 18 | 101 |
| 847135 | 34 | 19 | 79 |
| 847136 | 46 | 31 | 104 |
| 847137 | 21 | 17 | 71 |
| 847142 | 15 | 38 | 93 |
| 847143 | 5 | 12 | 133 |
| 847144 | 4 | 21 | 117 |
| 847935 | 31 | 31 | 96 |
| 847936 | 13 | 15 | 80 |
| 847938 | 15 | 28 | 95 |

For a subset of compounds, protein levels of Lamin A and progerin were measured by western blot analysis as described in Example 8. Protein expression levels were normalized to (3-actin and relative band intensity was measured. Expression levels are reported relative to those of mock-transfected HGPS cells.

TABLE 22
Expression levels of Lamin A protein and
Progerin protein in HGPS cells

| Compound ID | Lamin A protein | Progerin protein |
|---|---|---|
| Mock | 100 | 100 |
| 796553 | 39 | 58 |
| 847119 | 49 | 30 |
| 847120 | 78 | 45 |
| 847121 | 66 | 39 |
| 847122 | 74 | 63 |
| 847123 | 79 | 44 |
| 847124 | 64 | 76 |
| 847125 | 70 | 47 |
| 847126 | 69 | 52 |
| 847127 | 52 | 68 |
| 847129 | 47 | 61 |
| 847130 | 37 | 55 |
| 847131 | 31 | 56 |
| 847134 | 37 | 51 |
| 847135 | 37 | 49 |
| 817136 | 59 | 57 |
| 847137 | 40 | 43 |
| 847142 | 34 | 51 |
| 847143 | 23 | 41 |
| 847144 | 47 | 52 |
| 847936 | 25 | 44 |
| 847938 | 32 | 27 |

### Example 10 Activity of Modified Oligonucleotides Complementary to Human LMNA in a Mouse Model

Modified oligonucleotides complementary to human LMNA were tested in vivo in an HGPS mouse model. The compounds in the table below contain a single mismatch to SEQ ID NO: 1 and are fully complementary to SEQ ID NO: 4. The modified oligonucleotides in the table below comprise a 2′-methoxyethyl sugar moiety at each nucleoside and each internucleoside linkage is a phosphorothioate internucleoside linkage. Each cytosine residue is a 5-methyl cytosine.

TABLE 23
Modified Oligonucleotides

| | | SEQ ID | SEQ ID | SEQ ID | SEQ ID | SEQ |
|---|---|---|---|---|---|---|
| Compound | Sequence and Chemistry | NO: 1 | NO: 1 | NO: 4 | NO: 4 | ID |
| ID | Notation (5′ to 3′) | start site | stop site | start site | stop site | NO: |
| 845221 | AₑₛGₑₛAₑₛTₑₛGₑₛGₑₛGₑₛTₑₛ^{m}CₑₛAₑₛ | 24298 | 24317 | 2027 | 2046 | 154 |
| | ^{m}Cₑₛ^{m}Cₑₛ^{m}CₑₛAₑₛ^{m}Cₑₛ^{m}CₑₛTₑₛGₑₛGₑ | | | | | |
| 845222 | GₑₛAₑₛGₑₛAₑₛTₑₛGₑₛGₑₛGₑₛTₑₛ^{m}Cₑₛ | 24299 | 24318 | 2028 | 2047 | 155 |
| | ^{m}CₑₛAₑₛ^{m}Cₑₛ^{m}Cₑₛ^{m}CₑₛAₑₛ^{m}Cₑₛ^{m}Cₑₛ | | | | | |
| | TₑₛGₑ | | | | | |
| 845223 | AₑₛGₑₛGₑₛAₑₛGₑₛAₑₛTₑₛGₑₛGₑₛGₑₛ | 24301 | 24320 | 2030 | 2049 | 156 |
| | Tₑₛ^{m}Cₑₛ^{m}CₑₛAₑₛ^{m}Cₑₛ^{m}Cₑₛ^{m}CₑₛAₑₛ | | | | | |
| | ^{m}Cₑₛ^{m}Cₑ | | | | | |
| 845224 | Gₑₛ^{m}Cₑₛ^{m}CₑₛAₑₛGₑₛAₑₛGₑₛGₑₛAₑₛ | 24306 | 24325 | 2035 | 2054 | 157 |
| | GₑₛAₑₛTₑₛGₑₛGₑₛGₑₛTₑₛ^{m}Cₑₛ^{m}Cₑₛ | | | | | |
| | Aₑₛ^{m}Cₑ | | | | | |

LMNA G608G transgenic mice have been described by Varga, et. al., “Progressive vascular smooth muscle cell defects in a mouse model of Hutchinson-Gilford progeria syndrome,” PNAS Feb. 28, 2006 103 (9) 3250-3255, hereby incorporated by reference. Homozygous LMNA G608G mice were administered 150 mg/kg modified oligonucleotide by subcutaneous injection. Three weeks later, mice were sacrificed and tissues were harvested for analysis. One group of mice was administered PBS as a control. RT-PCR was used to measure LMNA isoforms as described in Examples 1 and 3, and protein levels were measured by western blot with the antibody ab40567 (Abcam), which detects an epitope that is common to lamin A, lamin C, prelamin A, and progerin. As shown in the tables below, modified oligonucleotides complementary to human LMNA are useful to reduce the amount of progerin mRNA (a LMNA transcription product associated with HGPS) and progerin protein in a HGPS mouse model.

TABLE 24
mRNA levels in vivo in various tissues

| | Liver | | | Heart | | | Aorta | | |
|---|---|---|---|---|---|---|---|---|---|
| | prelamin | | lamin | prelamin | | lamin | prelamin | | lamin |
| Compound | A | progerin | C | A | progerin | C | A | progerin | C |
| ID | mRNA | mRNA | mRNA | mRNA | mRNA | mRNA | mRNA | mRNA | mRNA |
| PBS | 100 | 100 | 100 | 100 | 100 | 100 | 100 | 100 | 100 |
| 637853 | 37 | 29 | 51 | 63 | 45 | 145 | 63 | 116 | 96 |
| 637856 | 28 | 30 | 67 | 53 | 56 | 163 | 76 | 75 | 99 |
| 845221 | 40 | 38 | 49 | 69 | 68 | 102 | 80 | 131 | 106 |
| 845222 | 71 | 63 | 58 | 71 | 63 | 90 | 76 | 138 | 102 |
| 845223 | 93 | 83 | 77 | 93 | 83 | 134 | 66 | 106 | 146 |
| 845224 | 23 | 31 | 83 | 55 | 43 | 142 | 59 | 61 | 164 |
| 847120 | 139 | 117 | 189 | 189 | 186 | 222 | 85 | 89 | 190 |
| 847125 | 32 | 23 | 189 | 32 | 38 | 189 | 37 | 34 | 187 |
| 847134 | 162 | 126 | 175 | 168 | 167 | 175 | 115 | 79 | 133 |
| 847143 | 5 | 17 | 214 | 15 | 26 | 214 | 17 | 22 | 250 |

TABLE 25
Protein levels in heart tissue

| Compound ID | Lamin A protein | Progerin protein |
|---|---|---|
| Mock | 100 | 100 |
| 637853 | 64 | 72 |
| 637856 | 66 | 75 |
| 845221 | 82 | 69 |
| 845222 | 46 | 51 |
| 845223 | 78 | 73 |
| 845224 | 69 | 64 |
| 847120 | 86 | 138 |
| 847125 | 42 | 46 |
| 847134 | 78 | 146 |
| 847143 | 39 | 51 |

### Example 11 Activity of an Oligomeric Compound Targeting Human LMNA in a Mouse Model

An oligomeric compound 958328 comprising the modified oligonucleotide 847143 with a 5′-C₁₆ conjugate represented by formula I below was synthesized and tested in a mouse model of HGPS.

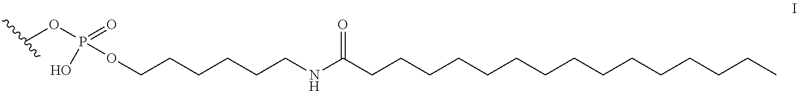 [SMILES: *OP(=O)(O)OCCCCCCNC(=O)CCCCCCCCCCCCCCC]

Homozygous LMNA G608G mice were treated with PBS, 17 mg/kg 958328, 50 mg/kg 958328, or 17 mg/kg scrambled control oligonucleotide SCRAM (884760; sequence GCTCATTTAGTCTGCCTGAT (SEQ ID NO: 159)). Each nucleoside of 884760 has a 2′-methoxyethyl modified sugar moiety, each internucleoside linkage is a phosphorothioate, and the compound comprises a 5′-C₁₆ conjugate identical to that on 958328. A total of 24 mice per treatment group were administered a dose 3x/week in the first week of treatment and two doses per week thereafter for a total of 12 weeks. For histopathology and mRNA analysis, a group of 6 mice per treatment were sacrificed after 3 months and another group of 6 mice were sacrificed at the completion of the study. RT-PCR was used to analyze LMNA mRNA isoforms as described in Examples 1 and 3 above. Data are reported relative to PBS-treated animals and were normalized to the mouse housekeeping gene mTfrc. For survival analysis, twelve mice per treatment as described above were monitored for the duration of the study.

TABLE 26
mRNA Levels

| | | | Liver | | Heart | | Aorta | |
|---|---|---|---|---|---|---|---|---|
| Compound | Dose | Duration | progerin | lamin C | progerin | lamin C | progerin | lamin C |
| ID | (mg/kg) | (months) | mRNA | mRNA | mRNA | mRNA | mRNA | mRNA |
| SCRAM | 50 | 3.5 | 57 | 79 | 72 | 68 | 106 | 114 |
| SCRAM | 50 | 5.5 | 50 | 97 | 98 | 71 | 93 | 73 |
| 958328 | 17 | 3.5 | 6.7 | 138 | 4.9 | 104 | 5.5 | 133 |
| 958328 | 17 | 5.5 | 0.23 | 281 | 3.0 | 176 | 6.0 | 164 |
| 958328 | 50 | 3.5 | 5.7 | 154 | 3.8 | 123 | 2.0 | 143 |
| 958328 | 50 | 5.5 | 0.19 | 222 | 0.8 | 169 | 0.71 | 145 |

Protein levels were analyzed by individual treated animal by western blots as described above. Each data point represents the data from one treated animal in the indicated treatment group for liver and heart tissues and 2-3 pooled aorta for the treatment group.

TABLE 27
Protein Levels

| | | | Liver | | Heart | | Aorta | |
|---|---|---|---|---|---|---|---|---|
| Compound | Dose | Duration | progerin | lamin C | progerin | lamin C | progerin | lamin C |
| ID | (mg/kg) | (months) | protein | protein | protein | protein | protein | protein |
| SCRAM | 50 | 5.5 | 25 | 37 | 67 | 101 | 100 | 47 |
| SCRAM | 50 | 5.5 | 48 | 97 | 62 | 131 | 69 | 54 |
| 958328 | 17 | 5.5 | 20 | 110 | 54 | 127 | 73 | 41 |
| 958328 | 17 | 5.5 | 23 | 113 | 52 | 122 | 76 | 48 |
| 958328 | 17 | 5.5 | n.d. | n.d. | 41 | 112 | n.d. | n.d. |
| 958328 | 50 | 5.5 | 6 | 152 | 27 | 116 | 44 | 54 |
| 958328 | 50 | 5.5 | 10 | 113 | 29 | 115 | 91 | 78 |
| 958328 | 50 | 5.5 | 3 | 83 | 27 | 104 | 44 | 54 |

Survival for groups of 12 mice was monitored. The median survival in days is reported in the table below. As shown in the table below, treatment with oligomeric compound 958328 prolongs survival as compared to control treated HGPS mice.

TABLE 28
Survival

| | | Median |
|---|---|---|
| Compound | Dose | Survival |
| ID | (mg/kg) | (days) |
| SCRAM | 50 | 232 |
| 958328 | 17 | 308 |
| 958328 | 50 | 275 |

Body weight for groups of 12 mice was monitored. The median body weight over the duration of the experiment is presented in the table below. Treatment with oligomeric compound 958328 reduces weight loss in HGPS mice.

TABLE 29
Body Weight (g)

| | Treatment | | |
|---|---|---|---|
| Day of | SCRAM, | 958328, | 958328, |
| Treatment | 50 mg/kg | 17 mg/kg | 50 mg/kg |
| 46 | 17.7 | 18.6 | 18.2 |
| 50 | 18.9 | 19.0 | 18.9 |
| 60 | 19.7 | 19.9 | 19.2 |
| 67 | 20.4 | 20.3 | 19.5 |
| 74 | 21.0 | 20.8 | 20.2 |
| 81 | 21.4 | 21.1 | 20.7 |
| 88 | 21.5 | 21.5 | 21.0 |
| 95 | 21.8 | 22.4 | 21.7 |
| 102 | 21.7 | 22.3 | 21.9 |
| 109 | 23.1 | 22.8 | 21.9 |
| 116 | 23.0 | 22.8 | 22.0 |
| 123 | 22.3 | 22.7 | 21.9 |
| 130 | 22.3 | 22.4 | 21.4 |
| 137 | 21.6 | 22.5 | 21.4 |
| 144 | 21.6 | 22.4 | 21.8 |
| 151 | 22.4 | 23.0 | 22.3 |
| 158 | 22.3 | 23.0 | 22.8 |
| 165 | 22.6 | 22.8 | 22.5 |
| 172 | 22.1 | 22.6 | 22.5 |
| 179 | 21.5 | 22.4 | 21.9 |
| 186 | 21.0 | 21.9 | 22.2 |
| 193 | 20.9 | 21.8 | 22.3 |
| 200 | 21.1 | 21.6 | 22.5 |
| 207 | 20.8 | 21.6 | 22.4 |
| 214 | 19.5 | 21.2 | 22.0 |
| 221 | 18.6 | 20.6 | 20.9 |
| 228 | 17.7 | 20.4 | 20.8 |
| 235 | 17.5 | 20.1 | 20.9 |
| 242 | 18.3 | 19.9 | 20.8 |
| 249 | 18.0 | 19.4 | 20.5 |
| 256 | 17.5 | 19.7 | 20.3 |
| 263 | 17.8 | 19.3 | 20.5 |
| 270 | 17.8 | 19.3 | 20.6 |
| 277 | 17.7 | 19.5 | 20.3 |
| 284 | 17.8 | 19.7 | 19.6 |
| 291 | 17.5 | 19.4 | 19.9 |
| 298 | n.d. | 19.8 | 19.2 |
| 305 | n.d. | 18.5 | 19.6 |
| 312 | n.d. | 19.4 | 19.7 |
| 319 | n.d. | 19.2 | 19.1 |
| 326 | n.d. | n.d. | 19.7 |
| 333 | n.d. | n.d. | 21.2 |
| 340 | n.d. | n.d. | 18.6 |

1. An oligomeric compound comprising a modified oligonucleotide consisting of 12 to 30 linked nucleosides and having a nucleobase sequence comprising at least 12, at least 13, at least 14, at least 15, at least 16, at least 17, or at least 18 contiguous nucleobases complementary to an equal length portion of nucleobases 24759-24791 of SEQ ID NO: 1, nucleobases 2176-2198 of SEQ ID NO: 2 or SEQ ID NO:4, or nucleobases 2062-2085 of SEQ ID NO: 3.

2. An oligomeric compound comprising a modified oligonucleotide consisting of 12 to 30 linked nucleosides and having a nucleobase sequence comprising at least 12, at least 13, at least 14, at least 15, or at least 16 any of the nucleobase sequences of SEQ ID 14-157.

3. An oligomeric compound comprising a modified oligonucleotide consisting of a modified oligonucleotide having a nucleobase sequence comprising at least 17, at least 18, at least 19, or at least 20 of any of the nucleobase sequences of SEQ ID 14-38, 75-101, or 132-157.

4. The oligomeric compound of claim 1, 2, or 3, wherein the modified oligonucleotide is at least 85%, at least 90%, at least 95%, or 100% complementary to SEQ ID NO: 1, SEQ ID NO: 2, SEQ ID NO: 3, or SEQ ID NO: 4 over the entire length of the modified oligonucleotide.

5. The oligomeric compound of any of claims 1-4, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, or at least 16 modified nucleoside comprising a modified sugar moiety.

6. The oligomeric compound of any of claims 1-5, wherein each nucleoside of the modified oligonucleotide comprises a modified sugar moiety.

7. The oligomeric compound of claim 5 or 6, wherein the modified sugar moiety is a 2′-methoxyethyl.

8. The oligomeric compound of any of claims 1-5, wherein the modified oligonucleotide comprises at least 1, at least 2, at least 3, at least 4, at least 5, at least 6, at least 7, at least 8, at least 9, at least 10, at least 11, at least 12, at least 13, at least 14, at least 15, or at least 16 modified nucleoside comprising a bicyclic sugar moiety having a 2′-4′ bridge.

9. The oligomeric compound of claim 8, wherein the 2′-4′ bridge is selected from —O—CH₂—; and —O—CH(CH₃)—.

10. The oligomeric compound of claim 9, wherein the 2′-4′ bridge is —O—CH(CH₃)—.

11. The oligomeric compound of claim 10, wherein each nucleoside is selected from a modified nucleoside comprising a bicyclic sugar moiety having a 2′-4′ bridge or an unmodified, β-D-2′-deoxyribose nucleoside.

12. The oligomeric compound of claim 11, wherein the 2′-4′ bridge is —O—CH(CH₃)—.

13. The oligomeric compound of any of claims 1-5, wherein the modified nucleotide has a modification pattern of (A)ₘ-(A—B—B)ₙ—(A)ₒ-(B)ₚ, wherein each A is a modified nucleoside comprising a bicyclic sugar moiety having a 2′-4′ bridge, each B is a non-bicyclic nucleoside, m is 0 or 1, n is from 5-9, o is 0 or 1, and p is 0 or 1, wherein if o is 0, p is also 0.

14. The oligomeric compound of claim 13, wherein the 2′-4′ bridge is —O—CH(CH₃)—.

15. The oligomeric compound of claim 13 or 14, wherein each B is a modified nucleoside comprising a 2′-methoxyethyl modified sugar moiety.

16. The oligomeric compound of claim 13 or 14, wherein each B is an unmodified, β-D-2′-deoxyribose nucleoside.

17. The oligomeric compound of any of claims 1-16, wherein at least one internucleoside linkage of the modified oligonucleotide is a modified internucleoside linkage.

18. The oligomeric compound of claim 17, wherein the modified internucleoside linkage is a phosphorothioate internucleoside linkage.

19. The oligomeric compound of any of claims 1-16, wherein each internucleoside linkage of the modified oligonucleotide is a modified internucleoside linkage.

20. The oligomeric compound of claim 19, wherein the modified internucleoside linkage is a phosphorothioate internucleoside linkage.

21. The oligomeric compound of any of claims 1-18, wherein at least one internucleoside linkage of the modified oligonucleotide is a phosphodiester internucleoside linkage.

22. The oligomeric compound of any of claims 1-18 and 21, wherein each internucleoside linkage of the modified oligonucleotide is either a phosphodiester internucleoside linkage or a phosphorothioate internucleoside linkage.

23. The oligomeric compound of any of claim 1-5, 7-12, or 17-20, wherein the modified oligonucleotide is a gapmer.

24. The oligomeric compound of any of claims 1-22, wherein the modified oligonucleotide is not a gapmer.

25. The oligomeric compound of any of claim 1-5, 8-10, or 13, wherein the modified oligonucleotide has a sugar motif selected from among: kkddkddkddkddkddkk, kddkddkddkddkddk, kkeekeekeekeekeeke, or keekeekeekeekeek, wherein “k” represents a modified nucleoside comprising a is —O—CH(CH₃)— 2′-4′ bridge, “d” represents a β-D-2′-deoxyribose, and “e” represents nucleoside comprising a 2′-methoxyethyl modified sugar moiety.

26. The oligomeric compound of any of claims 1-25, wherein the modified oligonucleotide consists of 12-18, 12-20, 14-18, 14-20, or 16-20 linked nucleosides.

27. The oligomeric compound of any of claims 1-26, wherein the modified oligonucleotide consists of 16, 17, 18, 19, or 20 linked nucleosides.

28. The oligomeric compound of any of claims 1-27, wherein at least one nucleobase of the modified oligonucleotide comprises a modified nucleobase.

29. The oligomeric compound of claim 28, wherein the modified nucleobase is a 5-methyl cytosine.

30. The oligomeric compound of claim 28, wherein the modified nucleobase is hypoxanthine.

31. The oligomeric compound of any of claims 1-5, wherein each nucleobase is selected from among adenine, guanine, cytosine, thymine, or 5-methyl cytosine.

32. The oligomeric compound of any of claims 1-5, wherein each nucleobase is selected from among adenine, guanine, cytosine, thymine, 5-methyl cytosine, or hypoxanthine.

33. The oligomeric compound of claim 32, wherein each nucleoside comprising adenine, guanine, cytosine, thymine, or 5-methyl cytosine comprises a 2′-modified sugar moiety, and wherein each nucleoside comprising hypoxanthine comprises a β-D-2′-deoxyribose.

34. The oligomeric compound of claim 33, wherein the modified sugar moiety is a 2′-methoxyethyl.

35. The oligomeric compound of any of claims 1-34, consisting of the modified oligonucleotide.

36. The oligomeric compound of any of claims 1-34, comprising a conjugate group comprising a conjugate moiety and a conjugate linker.

37. The oligomeric compound of claim 36, wherein the conjugate moiety comprises a lipophilic group.

38. The oligomeric compound of claim 37, wherein the conjugate moiety is selected from among: cholesterol, C10-C26 saturated fatty acid, C10-C26 unsaturated fatty acid, C10-C26 alkyl, triglyceride, tocopherol, or cholic acid.

39. The oligomeric compound of claim 38, wherein the conjugate moiety is a saturated fatty acid or an unsaturated fatty acid.

40. The oligomeric compound of claim 38, wherein the conjugate moiety is C16 alkyl.

41. The oligomeric compound of any of claims 36-40, wherein the conjugate linker consists of a single bond.

42. The oligomeric compound of any of claims 36-40, wherein the conjugate linker is cleavable.

43. The oligomeric compound of any of claims 36-40, wherein the conjugate linker comprises 1-3 linker nucleosides.

44. The oligomeric compound of claim 43, wherein the oligomeric compound comprises no more than 24 total linked nucleosides, including the modified oligonucleotide and linker nucleosides.

45. The oligomeric compound of any of claims 36-40, wherein the conjugate linker comprises a hexylamino group.

46. The oligomeric compound of any of claims 36-40, wherein the conjugate linker comprises a polyethylene glycol group.

47. The oligomeric compound of any of claims 36-40, wherein the conjugate linker comprises a triethylene group.

48. The oligomeric compound of any of claims 36-40, wherein the conjugate linker comprises a phosphate group.

49. The oligomeric compound of claim 36, wherein the conjugate group has formula I:

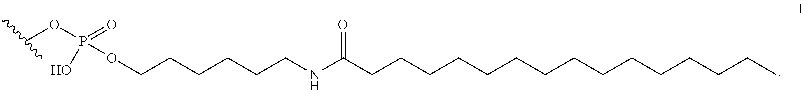 [SMILES: *OP(=O)(O)OCCCCCCNC(=O)CCCCCCCCCCCCCCC]

50. The oligomeric compound of any of claims 1-49, wherein the oligomeric compound is single-stranded.

51. An oligomeric duplex comprising any oligomeric compound of any of claims 1-49.

52. An antisense compound comprising or consisting of an oligomeric compound of any of claims 1-50 or an oligomeric duplex of claim 51.

53. A pharmaceutical composition comprising an oligomeric compound of any of claims 1-50, an oligomeric duplex of claim 51, or an antisense compound of claim 52, and at least one of a pharmaceutically acceptable carrier or diluent.

54. The pharmaceutical composition of claim 53, wherein the modified oligonucleotide is a sodium salt.

55. A method comprising administering to an animal the pharmaceutical composition of claim 53 or 54.

56. The method of claim 55, wherein the animal is a human.

57. A method of treating a disease associated with LMNA comprising administering to an individual having or at risk of developing a disease associated with LMNA a therapeutically effective amount of a pharmaceutical composition of claim 53 or 54.

58. The method of claim 56, wherein the disease is Hutchinson-Gilford Progeria Syndrome

59. The method of claim 57, wherein at least one symptom of Hutchinson-Gilford Progeria Syndrome is ameliorated.

60. The method of claim 59, wherein the symptom is weight loss.

61. The method of claim 59, wherein the symptoms is premature death.

62. A method comprising the co-administration of two or more oligomeric compounds of any of claims 1-50 to an individual.

63. A method comprising the concomitant administration of two or more oligomeric compounds of any of claims 1-50 to an individual.
